(19) United States
(12) Patent Application Publication
(10) Pub. No.: US 2011/0224210 A1
(43) Pub. Date: Sep. 15, 2011

(54) NOVEL BIS-AMIDES AS ANTI-MALARIAL AGENTS

(76) Inventors: Hamed Aissaoui, Allschwil (CH); Christoph Boss, Allschwill (CH); Olivier Corminboeuf, Allschwill (CH); Marie-Celine Frantz, Pittsburg, PA (US); Corinna Grisostomi, Allschwill (CH)

(21) Appl. No.: 13/130,151

(22) PCT Filed: Nov. 18, 2009
(86) PCT No.: PCT/IB2009/055147

§ 371 Date: May 19, 2011

(30) Foreign Application Priority Data
Nov. 19, 2008 (IB) PCT/IB2008/054858

(51) Int. Cl. A61K 31/5377; A61K 31/497; A61K 31/501; A61K 31/4545; A61K 31/4439; A61K 31/4418; C07D 401/14; C07D 413/12; C07D 401/12; C07D 211/70; A61P 33/06; A61P 33/02

(52) U.S. Cl. 514/235.5; 514/253.11; 514/256; 514/318; 514/339; 514/340; 514/343; 514/357; 544/131; 544/333; 544/360; 546/194; 546/271.4; 546/276.4; 546/277.7; 546/337

(57) ABSTRACT

The invention relates to novel bis-amide derivatives and their use as active ingredients in the preparation of pharmaceutical compositions. The invention also concerns related aspects including pharmaceutical compositions containing one or more of those compounds and their use as medicaments for the treatment or prevention of protozoal infections, such as especially malaria.

The invention relates to novel compounds of the formula I. The invention also concerns related aspects including processes for the preparation of the compounds, pharmaceutical compositions containing one or more compounds of the formula I and especially their use as medicaments to treat or prevent malaria infections or to treat or prevent other protozoal diseases like sleeping sickness, Chagas disease, amebiasis, giardiasis, trichomoniasis, toxoplasmosis, and leishmaniasis.

### BACKGROUND OF THE INVENTION

Numerous serious diseases affecting humans as well as domestic and livestock animal are caused by protozoal organisms such as kinetoplastida, apicomplexa, anaerobic protozoa, microsporidia and plasmodium, for example. The clinically most relevant of these diseases is malaria.

Malaria is one of the most serious and complex health problems affecting humanity in the 21^{st} century. The disease affects about 300 million people worldwide, killing 1 to 1.5 million people every year. Malaria is an infectious disease caused by four species of the protozoan parasite plasmodium, P. falciparum being the most severe of the four. All attempts to develop vaccines against P. falciparum have failed so far. Therefore, therapies and preventive measures against malaria are confined to drugs. Various classes of antimalarial drugs exist. The most widely used are the quinoline antimalarials, e.g. chloroquine which has been an especially effective drug for both prophylaxis and therapy. However, resistance to many of the currently available antimalarial drugs is spreading rapidly, threatening people in areas where malaria is endemic. Reports of multi-drug resistant strains of malaria parasites render the search for new antimalarial agents especially urgent. P. falciparum enters the human body by way of bites of the female anophelino mosquito (it may also be transmitted by blood transfusion from asymptotic donors; almost all infected blood components including red cells, platelet concentrates, white cells, cryoprecipitates and fresh plasma can transmit malaria). The plasmodium parasite initially populates the liver, and during later stages of the infectious cycle reproduces in red blood cells. During this stage, the parasite degrades hemoglobin and uses the degradation products as nutrients for growth.

The limitations of the current antiprotozoal chemotherapeutic arsenal underscore the need for new drugs in this therapeutic area. The present invention relates to the identification of novel low molecular weight, non-peptidic, non-quinoline compounds of formula I which are useful in the treatment and/or prevention of protozoal infections, especially in the treatment and/or prevention of malaria, in particular plasmodium falciparum malaria.

### DETAILED DESCRIPTION OF THE INVENTION

i) The present invention relates to novel compounds of the formula I:

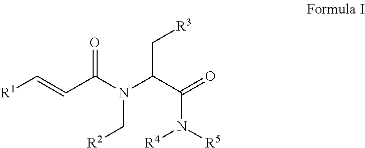

wherein
R¹ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, trifluoromethoxy, and amino, wherein the amino group is optionally mono- or di-substituted with (C₁-C₄)alkyl or mono-substituted with (C₁-C₄)alkyl-carbonyl; or R¹ represents aryl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl;
R² represents aryl or heteroaryl, wherein these two radicals can optionally be mono-, di-, tri-, or tetra-substituted, wherein the substituents are independently selected from the group consisting of halogen; (C₁-C₄)alkyl; (C₁-C₄)alkoxy; trifluoromethyl; trifluoromethoxy; heterocycloalkyl, that can optionally be mono-substituted on one nitrogen ring atom, if present, with (C₁-C₄)alkyl, or (C₁-C₄)alkyl-carbonyl; and aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
R³ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy; or R³ represents heterocycloalkyl that can optionally be mono-substituted on one nitrogen ring atom, if present, with (C₁-C₄)alkyl, cycloalkyl, (C₁-C₄)alkyl-carbonyl, or cycloalkyl-carbonyl; or R³ represents 2-oxo-oxazolidin-3-yl; or R³ represents 2,3-dioxo-2,3-dihydro-indol-1-yl that can optionally be mono-, di- or tri-substituted, wherein the substituents are independently selected from the group consisting of halogen, (C₁-C₄)alkyl, (C₁-C₄)alkoxy, trifluoromethyl, and trifluoromethoxy; and
R⁴ and R⁵, together with the nitrogen atom to which they are attached, form a morpholine ring; or together with the nitrogen atom to which they are attached, form the radicals 5,8-dihydro-6H-[1,7]naphthyridin-7-yl, 2,3-dihydro-1H-indol-1-yl, or 1,3-dihydro-1H-isoindol-2-yl, wherein these three radicals can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
or R⁴ and R⁵, together with the nitrogen atom to which they are attached, form a 3-amino-pyrrolidine ring, wherein the amino group is di-substituted with (C₁-C₄)alkyl; or together with the nitrogen atom to which they are attached, form a 3- or 4-substituted piperidine ring, wherein the substituent is selected from the group consisting of phenyl, benzyl, pyrrolidinomethyl, piperidinomethyl, amino di-substituted with (C₁-C₄)alkyl, and aminomethyl wherein the amino group is di-substituted with (C₁-C₄)alkyl;
or R⁴ represents hydrogen or (C₁-C₄)alkyl, and R⁵ represents 1-benzyl-pyrrolidin-3-yl or 1-aza-bicyclo[2.2.2]oct-3-yl;
or R⁴ represents (C₁-C₄)alkyl and R⁵ represents the following group:

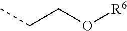

wherein R⁶ represents hydrogen, (C₁-C₄)alkyl, (C₃-C₄)alkenyl, cyanomethyl, carbamoylmethyl, cycloalkylmethyl, or 2-benzyloxy-ethyl; or R⁶ represents heteroaryl that can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; or R⁶ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cyano, trifluoromethyl, difluoromethoxy, and trifluoromethoxy;
or R⁴ represents hydrogen, (C₁-C₄)alkyl, or benzyl, and R⁵ represents the following group:

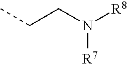

wherein R⁷ represents (C₁-C₄)alkyl; and R⁸ represents (C₁-C₄)alkyl or 4-methyl-3,4-dihydro-2H-benzo[1,4]oxazin-7-ylmethyl; or R⁸ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cycloalkyl, hydroxy, hydroxymethyl, cyano, trifluoromethyl, trifluoromethoxy, —O—(CH₂)₂—OH, —O—(CH₂)₃—N((C₁-C₄)alkyl)₂, and amino, wherein the amino group is mono- or di-substituted with substituents independently selected from (C₁-C₄)alkyl and hydroxy-(C₁-C₄)alkyl; or R⁸ represents arylmethyl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl; or R⁷ and R⁸, together with the nitrogen atom to which they are attached, form a piperidine, morpholine, or azepane ring;
or R⁴ represents (C₁-C₄)alkyl and R⁵ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
or R⁴ represents (C₁-C₄)alkyl and R⁵ represents the following group:

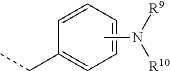

wherein the amino group can be in position 2, 3 or 4; R⁹ represents hydrogen, phenyl, or (C₁-C₄)alkyl; and R¹⁰ represents (C₁-C₄)alkyl, —(CH₂)₂—O—(C₁-C₄)alkyl, (C₁-C₄)alkyl-carbonyl, cycloalkyl-carbonyl, or benzoyl; or R⁹ and R¹⁰, together with the nitrogen atom to which they are attached, form a pyrrolidin-2-one or a piperidin-2-one ring.

The general terms used hereinbefore and hereinafter preferably have, within this disclosure, the following meanings, unless otherwise indicated:

The term (C₁-C₄)alkyl, alone or in combination with other groups, means saturated, straight or branched chain groups with one to four carbon atoms, preferably one to three carbon atoms, i.e. methyl, ethyl, n-propyl, iso-propyl, n-butyl, iso-butyl, sec-butyl, and tert-butyl. The methyl, ethyl and isopropyl groups are preferred.

The term (C₁-C₄)alkoxy, alone or in combination with other groups, refers to an R—O— group, wherein R is a (C₁-C₄)alkyl, i.e. methoxy, ethoxy, n-propoxy, iso-propoxy, n-butoxy, iso-butoxy, sec-butoxy, and tert-butoxy. The methoxy group is a preferred group.

The term (C₃-C₄)alkenyl, alone or in combination with other groups, means straight or branched chain groups comprising an olefinic bond and consisting of three to four carbon atoms, such as especially allyl.

The term (C₁-C₂)alkylenedioxy refers to methylenedioxy and 1,2-ethylenedioxy. If R¹ or R⁸ represent aryl or arylmethyl, respectively, wherein two adjacent carbon ring atoms of the aryl moiety are substituted with (C₁-C₂)alkylenedioxy, this means that methylenedioxy or 1,2-ethylenedioxy is attached via its oxygen atoms to the two adjacent carbon ring atoms of the aryl moiety, to form, together with the two adjacent carbon ring atoms, a 5- or 6-membered ring, respectively.

The term halogen means fluorine, chlorine, bromine or iodine, preferably fluorine, chlorine, or bromine.

The term cycloalkyl, alone or in combination with other groups, means a saturated cyclic hydrocarbon ring system with 3 to 7 carbon atoms, i.e. cyclopropyl, cyclobutyl, cyclopentyl, cyclohexyl, and cycloheptyl. The cyclopropyl group is a preferred group.

The term aryl, alone or in combination with other groups, relates to a phenyl or naphthyl group, preferably a phenyl group.

The term heteroaryl, alone or in combination with other groups, means a 5- to 10-membered monocyclic or bicyclic aromatic ring containing up to three, i.e. 1, 2, or 3, ring heteroatoms independently selected from oxygen, nitrogen, and sulfur. Examples of such heteroaryl groups are furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidyl, pyridazinyl, pyrazinyl, indolyl, isoindolyl, benzofuranyl, isobenzofuranyl, benzothiophenyl, indazolyl, benzimidazolyl, benzoxazolyl, benzoisoxazolyl, benzothiazolyl, benzotriazolyl, benzoxadiazolyl, benzothiadiazolyl, quinolinyl, isoquinolinyl, naphthyridinyl, cinnolinyl, quinazolinyl, quinoxalinyl, and phthalazinyl.

The term heterocycloalkyl, alone or in combination with other groups, means a 4-, 5-, or 6-membered saturated cyclic hydrocarbon ring system containing up to three, i.e. 1, 2, or 3, ring heteroatoms independently selected from oxygen, nitrogen, and sulfur. Examples of such heterocycloalkyl groups are pyrrolidinyl, piperidyl, morpholinyl, and piperazinyl.

ii) A further embodiment of the invention relates to compounds of the formula I according to embodiment i), wherein the carbon atom to which —CH₂—R³ is attached is in the (S)-configuration:

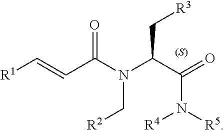

iii) A further embodiment of the invention relates to compounds of the formula I according to embodiment i) or ii), wherein:

R¹ represents mono-substituted aryl or mono-substituted heteroaryl, wherein the substituent is selected from the group consisting of halogen, (C₁-C₄)alkyl, (C₁-C₄)alkoxy, cycloalkyl, trifluoromethyl, and trifluoromethoxy.

iv) A further embodiment of the invention relates to compounds of the formula I according to embodiment iii), wherein:

R¹ represents mono-substituted aryl or mono-substituted heteroaryl, wherein the substituent is selected from the group consisting of chlorine, methyl, methoxy, and trifluoromethyl.

v) A further embodiment of the invention relates to compounds of the formula I according to any one of embodiments i) to iv), wherein:

R² represents mono-substituted aryl or mono-substituted heteroaryl, wherein the substituent is selected from the group consisting of halogen, (C₁-C₄)alkyl, (C₁-C₄)alkoxy, trifluoromethyl, trifluoromethoxy, aryl, heteroaryl, and heterocycloalkyl wherein the heterocycloalkyl can optionally be mono-substituted on one nitrogen ring atom, if present, with (C₁-C₄)alkyl or (C₁-C₄)alkyl-carbonyl.

vi) A further embodiment of the invention relates to compounds of the formula I according to any one of embodiments i) to v), wherein:

R³ represents phenyl, morpholin-4-yl, pyrrol-1-yl, or 1-methyl-1H-pyrazol-3-yl.

vii) A further embodiment of the invention relates to compounds of the formula I according to any one of embodiments i) to vi), wherein:

R⁴ and R⁵, together with the nitrogen atom to which they are attached, form a 4-substituted piperidine ring, wherein the substituent is phenyl or benzyl.

viii) A further embodiment of the invention relates to compounds of the formula I according to any one of embodiments i) to vi), wherein:

R⁴ represents (C₁-C₄)alkyl and R⁵ represents the following group:

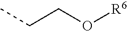

wherein R⁶ represents hydrogen, (C₁-C₄)alkyl, (C₃-C₄)alkenyl, cyanomethyl, carbamoylmethyl, cycloalkylmethyl, or 2-benzyloxy-ethyl; or R⁶ represents heteroaryl that can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; or R⁶ represents arylmethyl or heteroarylmethyl, wherein aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cyano, trifluoromethyl, difluoromethoxy, and trifluoromethoxy;
or R⁴ represents (C₁-C₄)alkyl and R⁵ represents the following group:

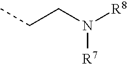

wherein R⁷ represents (C₁-C₄)alkyl; and R⁸ represents (C₁-C₄)alkyl or 4-methyl-3,4-dihydro-2H-benzo[1,4]oxazin-7-ylmethyl; or R⁸ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cycloalkyl, hydroxy, hydroxymethyl, cyano, trifluoromethyl, trifluoromethoxy, —O—(CH₂)₂—OH, —O—(CH₂)₃—N((C₁-C₄)alkyl)₂, and amino, wherein the amino group is mono- or di-substituted with substituents independently selected from (C₁-C₄)alkyl and hydroxy-(C₁-C₄)alkyl; or R⁸ represents arylmethyl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl;
or R⁴ represents (C₁-C₄)alkyl and R⁵ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
or R⁴ represents (C₁-C₄)alkyl and R⁵ represents the following group:

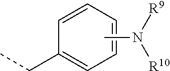

wherein the amino group can be in position 2, 3 or 4; R⁹ represents hydrogen, phenyl, or (C₁-C₄)alkyl; and R¹⁰ represents (C₁-C₄)alkyl, —(CH₂)₂—O—(C₁-C₄)alkyl, (C₁-C₄)alkyl-carbonyl, cycloalkyl-carbonyl, or benzoyl; or R⁹ and R¹⁰, together with the nitrogen atom to which they are attached, form a pyrrolidin-2-one or a piperidin-2-one ring.

ix) In another embodiment, the present invention relates to compounds of formula I according to embodiment i) wherein:

R¹ represents phenyl, pyridyl, pyrimidyl, pyridazinyl, pyrazolyl, oxazolyl, thiazolyl, imidazolyl, isoxazolyl, or thiadiazolyl, wherein these radicals can optionally be mono-, di-, or tri-substituted, wherein the substituents are independently selected from the group consisting of halogen, (C₁-C₄)alkyl such as methyl, (C₁-C₄)alkoxy such as methoxy, and trifluoromethyl;
R² represents phenyl or pyridyl, wherein these two radicals can optionally be mono-substituted (especially in para-position), wherein the substituent is selected from the group consisting of (C₁-C₄)alkyl such as ethyl, morpholin-4-yl, 4-acetyl-piperazin-1-yl, pyridyl, and pyrimidyl such as pyrimidin-5-yl;
R³ represents phenyl, pyrimidyl, imidazolyl, pyrrolyl, isoxazolyl, or pyrazolyl, wherein these radicals can optionally be mono-substituted with (C₁-C₄)alkyl such as methyl; or R³ represents pyrrolidinyl such as pyrrolidin-1-yl, morpholinyl such as morpholin-4-yl, or piperazinyl that can optionally be mono-substituted on one nitrogen ring atom with (C₁-C₄)alkyl such as 4-methyl-piperazin-1-yl; or R³ represents 2-oxo-oxazolidin-3-yl or 2,3-dioxo-2,3-dihydro-indol-1-yl; and
R⁴ and R⁵, together with the nitrogen atom to which they are attached, form a morpholine ring; or together with the nitrogen atom to which they are attached, form the radicals 5,8-dihydro-6H-[1,7]naphthyridin-7-yl, 2,3-dihydro-1H-indol-1-yl, or 1,3-dihydro-1H-isoindol-2-yl; or R⁴ and R⁵, together with the nitrogen atom to which they are attached, form a 3-amino-pyrrolidine ring, wherein the amino group is di-substituted with (C₁-C₄)alkyl such as methyl; or together with the nitrogen atom to which they are attached, form a 4-substituted piperidine ring, wherein the substituent is selected from the group consisting of phenyl, benzyl, pyrrolidinomethyl, amino di-substituted with (C₁-C₄)alkyl such as dimethylamino, and aminomethyl wherein the amino group is di-substituted with (C₁-C₄)alkyl such as dimethylaminomethyl;
or R⁴ represents hydrogen or (C₁-C₄)alkyl such as methyl, and R⁵ represents 1-benzyl-pyrrolidin-3-yl or 1-aza-bicyclo[2.2.2]oct-3-yl;
or R⁴ represents (C₁-C₄)alkyl such as methyl and R⁵ represents the following group:

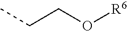

wherein R⁶ represents hydrogen, (C₁-C₄)alkyl such as methyl or ethyl, (C₃-C₄)alkenyl such as allyl, cyanomethyl, carbamoylmethyl, cycloalkylmethyl such as cyclopropylmethyl, or 2-benzyloxy-ethyl; or R⁶ represents pyrimidyl such as pyrimidin-2-yl; or R⁶ represents benzyl, pyridylmethyl, furanylmethyl, isoxazolylmethyl, or benzotriazolylmethyl such as benzotriazol-5-ylmethyl, wherein these radicals can optionally be mono- or di-substituted at the ring(s), wherein the substituents are independently selected from the group consisting of halogen, (C₁-C₄)alkyl such as methyl, (C₁-C₄)alkoxy such as methoxy, cyano, trifluoromethyl, difluoromethoxy, and trifluoromethoxy;
or R⁴ represents hydrogen, (C₁-C₄)alkyl such as methyl, or benzyl, and R⁵ represents the following group:

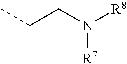

wherein R⁷ represents (C₁-C₄)alkyl such as methyl, isopropyl or n-butyl; and R⁸ represents (C₁-C₄)alkyl such as methyl, isopropyl or n-butyl, or 4-methyl-3,4-dihydro-2H-benzo[1,4]oxazin-7-ylmethyl; or R⁸ represents benzyl, pyridylmethyl, pyrimidylmethyl such as pyrimidin-5-ylmethyl, furanylmethyl, thienylmethyl, thiazolylmethyl, or imidazolylmethyl, wherein these radicals can optionally be mono-, di-, or tri-substituted at the ring, wherein the substituents are independently selected from the group consisting of halogen, (C₁-C₄)alkyl such as methyl, (C₁-C₄)alkoxy such as methoxy or isopropoxy, hydroxy, hydroxymethyl, cyano, trifluoromethyl, —O—(CH₂)₂—OH, —O—(CH₂)₃—N((C₁-C₄)alkyl)₂ such as —O—(CH₂)₃—N(CH₃)₂, and amino, wherein the amino group is di-substituted with substituents independently selected from (C₁-C₄)alkyl such as methyl or ethyl, and hydroxy-(C₁-C₄)alkyl such as 2-hydroxy-ethyl; or R⁸ represents phenylmethyl wherein two adjacent carbon ring atoms of the phenyl moiety are substituted with (C₁-C₂)alkylenedioxy such as benzo[1,3]dioxol-5-ylmethyl; or R⁷ and R⁸, together with the nitrogen atom to which they are attached, form a piperidine, morpholine, or azepane ring;
or R⁴ represents (C₁-C₄)alkyl such as methyl and R⁵ represents phenylmethyl, wherein the phenyl moiety is mono-substituted with (C₁-C₄)alkoxy such as methoxy;
or R⁴ represents (C₁-C₄)alkyl such as methyl and R⁵ represents the following group:

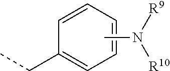

wherein the amino group is in position 4; R⁹ represents hydrogen or phenyl; and R¹⁰ represents —(CH₂)₂—O—(C₁-C₄)alkyl such as —(CH₂)₂—O—CH₃, (C₁-C₄)alkyl-carbonyl such as acetyl, cycloalkyl-carbonyl such as cyclopropylcarbonyl, or benzoyl; or R⁹ and R¹⁰, together with the nitrogen atom to which they are attached, form a pyrrolidin-2-one or a piperidin-2-one ring.

x) In another embodiment, the present invention relates to compounds of formula I according to embodiment i) wherein:

R¹ represents phenyl, pyridyl, pyrimidyl or pyridazinyl, wherein these four radicals are mono-substituted, wherein the substituent is selected from the group consisting of halogen, (C₁-C₄)alkyl such as especially methyl, (C₁-C₄)alkoxy such as especially methoxy, and trifluoromethyl; or R¹ represents 1-methyl-1H-pyrazol-3-yl, 1,5-dimethyl-1H-pyrazol-4-yl, 2,5-dimethyl-2H-pyrazol-3-yl, 1,3,5-trimethyl-1H-pyrazol-4-yl, 2-methyl-thiazol-4-yl, 2,4-dimethyl-thiazol-5-yl, 5-methyl-isoxazol-3-yl, 3,5-dimethyl-isoxazol-4-yl, 2,5-dimethyl-oxazol-4-yl, 2,3-dimethyl-3H-imidazol-4-yl, or [1,2,3]thiadiazol-4-yl;
R² represents phenyl or pyridyl, wherein these two radicals can optionally be mono-substituted (especially in para-position) with (C₁-C₄)alkyl such as especially ethyl, pyridyl, pyrimidyl such as especially pyrimidin-5-yl, morpholinyl such as especially morpholin-4-yl, or piperazinyl which is mono-substituted on one nitrogen ring atom with (C₁-C₄)alkyl-carbonyl such as especially 4-acetyl-piperazin-1-yl;
R³ represents phenyl, morpholinyl such as morpholin-4-yl, pyrrolyl such as pyrrol-1-yl, or 1-methyl-1H-pyrazol-3-yl, such as especially phenyl or morpholin-4-yl; and
R⁴ and R⁵, together with the nitrogen atom to which they are attached, form a morpholine ring; or together with the nitrogen atom to which they are attached, form the radicals 5,8-dihydro-6H-[1,7]naphthyridin-7-yl, 2,3-dihydro-1H-indol-1-yl, or 1,3-dihydro-1H-isoindol-2-yl;
or R⁴ and R⁵, together with the nitrogen atom to which they are attached, form a 3-amino-pyrrolidine ring, wherein the amino group is di-substituted with (C₁-C₄)alkyl such as especially 3-dimethylamino-pyrrolidin-1-yl; or together with the nitrogen atom to which they are attached, form a 3- or 4-substituted piperidine ring (especially 4-substituted), wherein the substituent is independently selected from the group consisting of phenyl, benzyl, pyrrolidinomethyl, amino di-substituted with (C₁-C₄)alkyl such as especially dimethylamino, and aminomethyl wherein the amino group is di-substituted with (C₁-C₄)alkyl such as especially dimethylaminomethyl;
or R⁴ represents (C₁-C₄)alkyl such as especially methyl, and R⁵ represents 1-benzyl-pyrrolidin-3-yl;
or R⁴ represents (C₁-C₄)alkyl such as especially methyl, and R⁵ represents the following group:

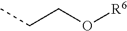

wherein R⁶ represents hydrogen, (C₁-C₄)alkyl such as especially methyl, (C₃-C₄)alkenyl such as especially allyl, cyanomethyl, carbamoylmethyl, cycloalkylmethyl such as especially cyclopropylmethyl, or 2-benzyloxy-ethyl; or R⁶ represents pyrimidyl such as especially pyrimidin-2-yl; or R⁶ represents phenylmethyl or pyridylmethyl, wherein the phenyl or pyridyl moiety can optionally be mono- or di-substituted, wherein the substituents are independently selected from the group consisting of halogen, (C₁-C₄)alkyl such as especially methyl, (C₁-C₄)alkoxy such as especially methoxy, cyano, difluoromethoxy, and trifluoromethoxy; or R⁶ represents 5-trifluoromethyl-furan-3-ylmethyl, 5-methyl-isoxazol-3-ylmethyl, or 1-methyl-1H-benzotriazol-5-ylmethyl;
or R⁴ represents (C₁-C₄)alkyl such as especially methyl, and R⁵ represents the following group:

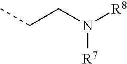

wherein R⁷ represents (C₁-C₄)alkyl such as especially methyl; and R⁸ represents (C₁-C₄)alkyl such as especially methyl; or R⁸ represents phenylmethyl or pyridylmethyl, wherein the phenyl or pyridyl moiety can optionally be mono-, di-, or tri-substituted, wherein the substituents are independently selected from the group consisting of halogen, (C₁-C₄)alkyl such as especially methyl, (C₁-C₄)alkoxy such as especially methoxy, hydroxy, cyano, trifluoromethyl, —O—(CH₂)₂—OH, —O—(CH₂)₃—N((C₁-C₄)alkyl)₂ such as especially —O—(CH₂)₃—N(CH₃)₂, and amino, wherein the amino group is di-substituted wherein the substituents are independently selected from (C₁-C₄)alkyl and hydroxy-(C₁-C₄)alkyl such as diethylamino or N-(2-hydroxy-ethyl)-N-methyl-amino; or R⁸ represents pyrimidylmethyl such as especially pyrimidin-5-ylmethyl; or R⁸ represents furan-2-ylmethyl, furan-3-ylmethyl, 5-bromo-furan-2-ylmethyl, 5-hydroxymethyl-furan-2-ylmethyl, thiophen-2-ylmethyl, thiophen-3-ylmethyl, 5-chloro-thiophen-2-ylmethyl, thiazol-2-ylmethyl, 3H-imidazol-4-ylmethyl, or 4-methyl-3,4-dihydro-2H-benzo[1,4]oxazin-7-ylmethyl; or R⁸ represents phenylmethyl, wherein two adjacent carbon ring atoms of the phenyl moiety are substituted with (C₁-C₂)alkylenedioxy, such as especially benzo[1,3]dioxol-5-ylmethyl;
or R⁴ represents (C₁-C₄)alkyl such as especially methyl, and R⁵ represents phenylmethyl, wherein the phenyl moiety is mono-substituted with (C₁-C₄)alkoxy such as especially methoxy;
or R⁴ represents (C₁-C₄)alkyl such as especially methyl, and R⁵ represents the following group:

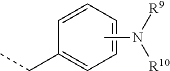

wherein the amino group can be in position 2, 3, or 4 (especially in position 4); R⁹ represents hydrogen or phenyl, such as especially hydrogen; and R¹⁰ represents —(CH₂)₂—O—(C₁-C₄)alkyl such as especially —(CH₂)₂—O—CH₃, (C₁-C₄)alkyl-carbonyl such as especially acetyl, cycloalkyl-carbonyl such as especially cyclopropyl-carbonyl, or benzoyl; or R⁹ and R¹⁰, together with the nitrogen atom to which they are attached, form a pyrrolidin-2-one or a piperidin-2-one ring.

The compounds of formula I may contain one or more stereogenic or asymmetric centers, such as one or more asymmetric carbon atoms. The compounds of formula I may thus be present as mixtures of stereoisomers or preferably as pure stereoisomers. Mixtures of stereoisomers may be separated in a manner known to a person skilled in the art.

Where the plural form is used for compounds, salts, pharmaceutical compositions, diseases and the like, this is intended to mean also a single compound, salt, or the like.

Any reference hereinbefore or hereinafter to a compound of formula I is to be understood as referring also to salts, especially pharmaceutically acceptable salts, of a compound of formula I, as appropriate and expedient.

The term “pharmaceutically acceptable salts” refers to non-toxic, inorganic or organic acid and/or base addition salts. Reference can be made to “Salt selection for basic drugs”, Int. J. Pharm. 1986, 33, 201-17.

Examples of preferred compounds of formula I are selected from the group consisting of:
(S)—N-[1-Benzyl-2-(4-dimethylamino-piperidin-1-yl)-2-oxo-ethyl]-N-(4-pyridin-2-yl-benzyl)-3-(4-trifluoromethyl-phenyl)-acrylamide;
(S)—N-[1-Benzyl-2-(4-dimethylamino-piperidin-1-yl)-2-oxo-ethyl]-N-(4-pyridin-2-yl-benzyl)-3-(6-trifluoromethyl-pyridin-3-yl)-acrylamide;
(S)—N-[1-Benzyl-2-(4-dimethylamino-piperidin-1-yl)-2-oxo-ethyl]-N-(4-pyridin-2-yl-benzyl)-3-(1,3,5-trimethyl-1H-pyrazol-4-yl)-acrylamide;
(S)—N-[1-Benzyl-2-(4-dimethylamino-piperidin-1-yl)-2-oxo-ethyl]-N-(4-pyridin-4-yl-benzyl)-3-(1,3,5-trimethyl-1H-pyrazol-4-yl)-acrylamide;
(S)—N-[1-Benzyl-2-(4-dimethylamino-piperidin-1-yl)-2-oxo-ethyl]-3-(2,4-dimethyl-thiazol-5-yl)-N-(4-pyridin-4-yl-benzyl)-acrylamide;
(S)—N-[1-Benzyl-2-(4-dimethylamino-piperidin-1-yl)-2-oxo-ethyl]-3-(2,5-dimethyl-oxazol-4-yl)-N-(4-pyridin-4-yl-benzyl)-acrylamide;
(S)—N-[1-Benzyl-2-(4-dimethylamino-piperidin-1-yl)-2-oxo-ethyl]-3-(6-methyl-pyridin-3-yl)-N-(4-pyridin-4-yl-benzyl)acrylamide;
(S)—N-[1-Benzyl-2-(4-dimethylamino-piperidin-1-yl)-2-oxo-ethyl]-3-(5-methyl-pyridin-2-yl)-N-(4-pyridin-4-yl-benzyl)-acrylamide;
(S)—N-[1-Benzyl-2-(4-dimethylamino-piperidin-1-yl)-2-oxo-ethyl]-3-(6-methoxy-pyridin-3-yl)-N-(4-pyridin-4-yl-benzyl)-acrylamide;
(S)—N-[1-Benzyl-2-(4-dimethylamino-piperidin-1-yl)-2-oxo-ethyl]-N-(4-pyridin-4-yl-benzyl)-3-(5-trifluoromethyl-pyridin-2-yl)-acrylamide;
(S)—N-[1-Benzyl-2-(4-dimethylamino-piperidin-1-yl)-2-oxo-ethyl]-3-(1,5-dimethyl-1H-pyrazol-4-yl)-N-(4-pyridin-4-yl-benzyl)-acrylamide;
(S)—N-[1-Benzyl-2-(4-benzyl-piperidin-1-yl)-2-oxo-ethyl]-N-pyridin-2-ylmethyl-3-(4-trifluoromethyl-phenyl)-acrylamide;
(S)—N-Benzyl-N-[1-benzyl-2-(4-benzyl-piperidin-1-yl)-2-oxo-ethyl]-3-(4-trifluoromethyl-phenyl)-acrylamide;
(S)—N-[1-Benzyl-2-(4-benzyl-piperidin-1-yl)-2-oxo-ethyl]-N-(4-pyridin-2-yl-benzyl)-3-(4-trifluoromethyl-phenyl)-acrylamide;
(S)—N-[1-Benzyl-2-(4-benzyl-piperidin-1-yl)-2-oxo-ethyl]-N-(4-pyridin-3-yl-benzyl)-3-(6-trifluoromethyl-pyridin-3-yl)-acrylamide;
(S)—N-Benzyl-N-[1-benzyl-2-(4-benzyl-piperidin-1-yl)-2-oxo-ethyl]-3-(6-trifluoromethyl-pyridin-3-yl)-acrylamide;
(S)—N-[1-Benzyl-2-oxo-2-(4-phenyl-piperidin-1-yl)-ethyl]-N-(4-pyrimidin-5-yl-benzyl)-3-(6-trifluoromethyl-pyridin-3-yl)-acrylamide;
(S)—N-[1-Benzyl-2-oxo-2-(4-phenyl-piperidin-1-yl)-ethyl]-N-(4-pyridin-2-yl-benzyl)-3-(6-trifluoromethyl-pyridin-3-yl)-acrylamide;
(S)—N-[1-Benzyl-2-(4-dimethylaminomethyl-piperidin-1-yl)-2-oxo-ethyl]-N-(4-pyridin-2-yl-benzyl)-3-(6-trifluoromethyl-pyridin-3-yl)-acrylamide;
(S)—N-[1-Benzyl-2-oxo-2-(4-pyrrolidin-1-ylmethyl-piperidin-1-yl)-ethyl]-N-(4-pyridin-2-yl-benzyl)-3-(4-trifluoromethyl-phenyl)-acrylamide;
N—-[(S)-1-Benzyl-2-((R)-3-dimethylamino-pyrrolidin-1-yl)-2-oxo-ethyl]-N-(4-pyridin-2-yl-benzyl)-3-(4-trifluoromethyl-phenyl)-acrylamide;
N—-[(S)-1-Benzyl-2-((S)-3-dimethylamino-pyrrolidin-1-yl)-2-oxo-ethyl]-N-(4-pyridin-2-yl-benzyl)-3-(4-trifluoromethyl-phenyl)-acrylamide;
N—-[(S)-1-Benzyl-2-((R)-3-dimethylamino-pyrrolidin-1-yl)-2-oxo-ethyl]-N-(4-pyridin-2-yl-benzyl)-3-(6-trifluoromethyl-pyridin-3-yl)-acrylamide;
N—-[(S)-1-Benzyl-2-((S)-3-dimethylamino-pyrrolidin-1-yl)-2-oxo-ethyl]-N-(4-pyridin-2-yl-benzyl)-3-(6-trifluoromethyl-pyridin-3-yl)-acrylamide;
(S)—N-[1-Benzyl-2-(2,3-dihydro-indol-1-yl)-2-oxo-ethyl]-N-(4-pyrimidin-5-yl-benzyl)-3-(4-trifluoromethyl-phenyl)-acrylamide;
(S)—N-[1-Benzyl-2-(1,3-dihydro-isoindol-2-yl)-2-oxo-ethyl]-N-(4-pyridin-2-yl-benzyl)-3-(6-trifluoromethyl-pyridin-3-yl)-acrylamide;
(S)—N-{1-[(2-Methoxy-ethyl)-methyl-carbamoyl]-2-phenyl-ethyl}-N-(4-morpholin-4-yl-benzyl)-3-(1,3,5-trimethyl-1H-pyrazol-4-yl)-acrylamide;
(S)-3-(2,4-Dimethyl-thiazol-5-yl)-N-{1-[(2-methoxy-ethyl)-methyl-carbamoyl]-2-phenyl-ethyl}-N-(4-morpholin-4-yl-benzyl)-acrylamide;
(S)—N-{1-[(2-Methoxy-ethyl)-methyl-carbamoyl]-2-phenyl-ethyl}-N-(4-morpholin-4-yl-benzyl)-3-(5-trifluoromethyl-pyridin-2-yl)-acrylamide;
(S)—N-{1-[(2-Methoxy-ethyl)-methyl-carbamoyl]-2-phenyl-ethyl}-3-(6-methoxy-pyridin-3-yl)-N-(4-morpholin-4-yl-benzyl)-acrylamide;
(S)-3-(2,5-Dimethyl-2H-pyrazol-3-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-N-(4-pyridin-2-yl-benzyl)-3-(1,3,5-trimethyl-1H-pyrazol-4-yl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(2-methyl-thiazol-4-yl)-N-(4-pyridin-2-yl-benzyl)-acrylamide;
(S)-3-(3,5-Dimethyl-isoxazol-4-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(6-methoxy-pyridazin-3-yl)-N-(4-pyridin-2-yl-benzyl)-acrylamide;
(S)—N-{1-[(2-Methoxy-ethyl)-methyl-carbamoyl]-2-phenyl-ethyl}-3-(6-methyl-pyridin-3-yl)-N-(4-pyridin-2-yl-benzyl)-acrylamide;
(S)-3-(6-Chloro-pyridin-3-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(5-methyl-pyridin-2-yl)-N-(4-pyridin-2-yl-benzyl)-acrylamide;
(S)—N-{1-[(2-Methoxy-ethyl)-methyl-carbamoyl]-2-phenyl-ethyl}-3-(6-methoxy-pyridin-3-yl)-N-(4-pyridin-2-yl-benzyl)-acrylamide;
(S)—N-{1-[(2-Methoxy-ethyl)-methyl-carbamoyl]-2-phenyl-ethyl}-N-(4-pyridin-2-yl-benzyl)-3-(2-trifluoromethyl-pyrimidin-5-yl)-acrylamide;
(S)—N-{1-[(2-Methoxy-ethyl)-methyl-carbamoyl]-2-phenyl-ethyl}-N-(4-pyridin-2-yl-benzyl)-3-(5-trifluoromethyl-pyridin-2-yl)-acrylamide;
(S)-3-(1,5-Dimethyl-1H-pyrazol-4-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(1-methyl-1H-pyrazol-3-yl)-N-(4-pyridin-4-yl-benzyl)-acrylamide;
(S)-3-(2,3-Dimethyl-3H-imidazol-4-yl)-N-{1-[(2-methoxy-ethyl)-methyl-carbamoyl]-2-phenyl-ethyl}-N-(4-pyridin-4-yl-benzyl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyridin-4-yl-benzyl)-acrylamide;
(S)—N-{1-[(2-Methoxy-ethyl)-methyl-carbamoyl]-2-phenyl-ethyl}-3-(2-methyl-thiazol-4-yl)-N-(4-pyridin-4-yl-benzyl)-acrylamide;
(S)—N-{1-[(2-Methoxy-ethyl)-methyl-carbamoyl]-2-phenyl-ethyl}-3-(5-methyl-isoxazol-3-yl)-N-(4-pyridin-4-yl-benzyl)-acrylamide;
(S)-3-(3,5-Dimethyl-isoxazol-4-yl)-N-{1-[(2-methoxy-ethyl)-methyl-carbamoyl]-2-phenyl-ethyl}-N-(4-pyridin-4-yl-benzyl)-acrylamide;
(S)-3-(2,5-Dimethyl-oxazol-4-yl)-N-{1-[(2-methoxy-ethyl)-methyl-carbamoyl]-2-phenyl-ethyl}-N-(4-pyridin-4-yl-benzyl)-acrylamide;
(S)—N-{1-[(2-Methoxy-ethyl)-methyl-carbamoyl]-2-phenyl-ethyl}-N-(4-pyridin-4-yl-benzyl)-3-[1,2,3]thiadiazol-4-yl-acrylamide;
(S)-3-(6-Chloro-pyridin-3-yl)-N-{1-[(2-methoxy-ethyl)-methyl-carbamoyl]-2-phenyl-ethyl}-N-(4-pyridin-4-yl-benzyl)-acrylamide;
(S)—N-{1-[(2-Methoxy-ethyl)-methyl-carbamoyl]-2-phenyl-ethyl}-3-(5-methyl-pyridin-2-yl)-N-(4-pyridin-4-yl-benzyl)-acrylamide;
(S)—N-{1-[(2-Methoxy-ethyl)-methyl-carbamoyl]-2-phenyl-ethyl}-3-(6-methoxy-pyridin-3-yl)-N-(4-pyridin-4-yl-benzyl)-acrylamide;
(S)—N-{1-[(2-Methoxy-ethyl)-methyl-carbamoyl]-2-phenyl-ethyl}-N-(4-pyridin-4-yl-benzyl)-3-(5-trifluoromethyl-pyridin-2-yl)-acrylamide;
(S)—N-[4-(4-Acetyl-piperazin-1-yl)-benzyl]-3-(2,5-dimethyl-2H-pyrazol-3-yl)-N-{1-[(2-methoxy-ethyl)-methyl-carbamoyl]-2-phenyl-ethyl}-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(1,3,5-trimethyl-1H-pyrazol-4-yl)-acrylamide;
(S)—N-[4-(4-Acetyl-piperazin-1-yl)-benzyl]-3-(2,4-dimethyl-thiazol-5-yl)-N-{1-[(2-methoxy-ethyl)-methyl-carbamoyl]-2-phenyl-ethyl}-acrylamide;
(S)—N-[4-(4-Acetyl-piperazin-1-yl)-benzyl]-3-(6-chloro-pyridin-3-yl)-N-{1-[(2-methoxy-ethyl)-methyl-carbamoyl]-2-phenyl-ethyl}-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(6-methoxy-pyridin-3-yl)-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(5-trifluoromethyl-pyridin-2-yl)-acrylamide;
(S)—N-{1-[(2-Methoxy-ethyl)-methyl-carbamoyl]-2-phenyl-ethyl}-3-(1-methyl-1H-pyrazol-3-yl)-N-(4-pyrimidin-5-yl-benzyl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyrimidin-5-yl-benzyl)-acrylamide;
(S)-3-(5-Chloro-pyridin-2-yl)-N-{1-[(2-methoxy-ethyl)-methyl-carbamoyl]-2-phenyl-ethyl}-N-(4-pyrimidin-5-yl-benzyl)-acrylamide;
(S)-3-(6-Chloro-pyridin-3-yl)-N-{1-[(2-methoxy-ethyl)-methyl-carbamoyl]-2-phenyl-ethyl}-N-(4-pyrimidin-5-yl-benzyl)-acrylamide;
(S)—N-{1-[(2-Methoxy-ethyl)-methyl-carbamoyl]-2-phenyl-ethyl}-3-(2-methoxy-pyrimidin-5-yl)-N-(4-pyrimidin-5-yl-benzyl)-acrylamide;
(S)—N-{1-[(2-Methoxy-ethyl)-methyl-carbamoyl]-2-phenyl-ethyl}-N-(4-pyrimidin-5-yl-benzyl)-3-(5-trifluoromethyl-pyridin-2-yl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyridin-3-yl-benzyl)-acrylamide;
(S)-3-(2,5-Dimethyl-oxazol-4-yl)-N-{1-[(2-methoxy-ethyl)-methyl-carbamoyl]-2-phenyl-ethyl}-N-(4-pyridin-3-yl-benzyl)-acrylamide;
(S)-3-(6-Chloro-pyridin-3-yl)-N-{1-[(2-methoxy-ethyl)-methyl-carbamoyl]-2-phenyl-ethyl}-N-(4-pyridin-3-yl-benzyl)-acrylamide;
(S)—N-{1-[(2-Methoxy-ethyl)-methyl-carbamoyl]-2-phenyl-ethyl}-3-(6-methoxy-pyridin-3-yl)-N-(4-pyridin-3-yl-benzyl)-acrylamide;
(S)—N-[1-Benzyl-2-(4-dimethylamino-piperidin-1-yl)-2-oxo-ethyl]-N-(6-morpholin-4-yl-pyridin-3-ylmethyl)-3-(4-trifluoromethyl-phenyl)-acrylamide;
(S)—N-[1-Benzyl-2-(4-benzyl-piperidin-1-yl)-2-oxo-ethyl]-N-(6-morpholin-4-yl-pyridin-3-ylmethyl)-3-(4-trifluoromethyl-phenyl)-acrylamide;
(S)—N-[1-Benzyl-2-(4-benzyl-piperidin-1-yl)-2-oxo-ethyl]-N-(6-morpholin-4-yl-pyridin-3-ylmethyl)-3-(6-trifluoromethyl-pyridin-3-yl)-acrylamide;
(S)—N-[1-Benzyl-2-(4-benzyl-piperidin-1-yl)-2-oxo-ethyl]-3-(4-methoxy-phenyl)-N-(6-morpholin-4-yl-pyridin-3-ylmethyl)-acrylamide;
(S)—N-[1-Benzyl-2-(4-benzyl-piperidin-1-yl)-2-oxo-ethyl]-N-(6-morpholin-4-yl-pyridin-3-ylmethyl)-3-p-tolyl-acrylamide;
(S)—N-(1-Benzyl-2-morpholin-4-yl-2-oxo-ethyl)-N-(6-morpholin-4-yl-pyridin-3-ylmethyl)-3-p-tolyl-acrylamide;
(S)—N-(1-Benzyl-2-morpholin-4-yl-2-oxo-ethyl)-3-(4-methoxy-phenyl)-N-(6-morpholin-4-yl-pyridin-3-ylmethyl)-acrylamide;
(S)—N-[1-Benzyl-2-(5,8-dihydro-6H-[1,7]naphthyridin-7-yl)-2-oxo-ethyl]-N-(4-pyridin-2-yl-benzyl)-3-(4-trifluoromethyl-phenyl)-acrylamide;
(S)—N-{1-[(2-Methoxy-ethyl)-methyl-carbamoyl]-2-phenyl-ethyl}-N-(6-morpholin-4-yl-pyridin-3-ylmethyl)-3-(4-trifluoromethyl-phenyl)-acrylamide;
(S)—N-{1-[(2-Methoxy-ethyl)-methyl-carbamoyl]-2-phenyl-ethyl}-N-(6-morpholin-4-yl-pyridin-3-ylmethyl)-3-p-tolyl-acrylamide;
(S)—N-{1-[(2-Methoxy-ethyl)-methyl-carbamoyl]-2-phenyl-ethyl}-3-(4-methoxy-phenyl)-N-(6-morpholin-4-yl-pyridin-3-ylmethyl)-acrylamide;
(S)—N-Benzyl-N-{1-[(2-methoxy-ethyl)-methyl-carbamoyl]-2-phenyl-ethyl}-3-p-tolyl-acrylamide;
(S)—N-(4-Ethyl-benzyl)-N-{1-[(2-methoxy-ethyl)-methyl-carbamoyl]-2-phenyl-ethyl}-3-p-tolyl-acrylamide;
(S)—N-{1-[(2-Methoxy-ethyl)-methyl-carbamoyl]-2-phenyl-ethyl}-N-pyridin-2-ylmethyl-3-p-tolyl-acrylamide;
(S)—N-{1-[(2-Methoxy-ethyl)-methyl-carbamoyl]-2-pyrrol-1-yl-ethyl}-N-(4-pyridin-2-yl-benzyl)-3-(4-trifluoromethyl-phenyl)-acrylamide;
N-[1-[(2-Methoxy-ethyl)-methyl-carbamoyl]-2-(1-methyl-1H-pyrazol-3-yl)-ethyl]-N-(4-pyridin-2-yl-benzyl)-3-(4-trifluoromethyl-phenyl)-acrylamide;
N-[1-[(2-Methoxy-ethyl)-methyl-carbamoyl]-2-(1-methyl-1H-pyrazol-4-yl)-ethyl]-N-(4-pyridin-4-yl-benzyl)-3-(4-trifluoromethyl-phenyl)-acrylamide;
N-[1-[(2-Methoxy-ethyl)-methyl-carbamoyl]-2-(1-methyl-1H-pyrazol-3-yl)-ethyl]-N-(4-pyridin-4-yl-benzyl)-3-(4-trifluoromethyl-phenyl)-acrylamide;
(S)—N-{1-[(2-Dimethylamino-ethyl)-methyl-carbamoyl]-2-phenyl-ethyl}-N-(4-pyridin-2-yl-benzyl)-3-(4-trifluoromethyl-phenyl)-acrylamide;
N-{1-[((S)-1-Benzyl-pyrrolidin-3-yl)-methyl-carbamoyl]-(S)-2-phenyl-ethyl}-N-(4-pyridin-2-yl-benzyl)-3-(4-trifluoromethyl-phenyl)-acrylamide;
N-{1-[((R)-1-Benzyl-pyrrolidin-3-yl)-methyl-carbamoyl]-(S)-2-phenyl-ethyl}-N-(4-pyridin-2-yl-benzyl)-3-(4-trifluoromethyl-phenyl)-acrylamide;
(S)—N-{1-[(2-Hydroxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-{1-[(2-hydroxy-ethyl)-methyl-carbamoyl]-2-phenyl-ethyl}-3-(4-trifluoromethyl-phenyl)-acrylamide;
(S)—N-{1-[(4-Methoxy-benzyl)-methyl-carbamoyl]-2-phenyl-ethyl}-N-(4-morpholin-4-yl-benzyl)-3-(6-trifluoromethyl-pyridin-3-yl)-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(4-trifluoromethyl-phenyl)-acrylamide;
(S)—N-{1-[(2-Methoxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-(1-{methyl-[2-(pyrimidin-2-yloxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-benzyloxy-ethyl)-methyl-carbamoyl]-2-phenyl-ethyl}-3-(4-trifluoromethyl-phenyl)-acrylamide;
(S)—N-{1-[(2-Benzyloxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-(1-{[2-(2,4-dimethyl-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(3-fluoro-4-methoxy-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(3-cyano-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(3-trifluoromethoxy-benzyloxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(3-methoxy-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(4-difluoromethoxy-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(1-methyl-1H-benzotriazol-5-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(pyridin-3-ylmethoxy)-ethyl]-carbamoyl}-2-phenyl-ethyl)-3-(4-trifluoromethyl-phenyl)-acrylamide;
(S)—N-(1-{[2-(3-Methoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-{1-[(2-Ethoxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-{1-[(2-ethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-(1-{[2-(2,4-dimethyl-benzyloxy)-ethyl]-methyl-carbamoyl}-2-phenyl-ethyl)-3-(4-trifluoromethyl-phenyl)-acrylamide;
(S)—N-(1-{[2-(2,4-Dimethyl-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(3-Fluoro-4-methoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(3-Cyano-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{Methyl-[2-(3-trifluoromethoxy-benzyloxy)-ethyl]-carbamoyl}-2-phenyl-ethyl)-N-(4-morpholin-4-yl-benzyl)-3-(4-trifluoromethyl-phenyl)-acrylamide;
(S)—N-(1-{[2-(3,5-Dimethoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(3-Methoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(4-Difluoromethoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{Methyl-[2-(1-methyl-1H-benzotriazol-5-ylmethoxy)-ethyl]-carbamoyl}-2-phenyl-ethyl)-N-(4-morpholin-4-yl-benzyl)-3-(4-trifluoromethyl-phenyl)-acrylamide;
(S)—N-(1-{Methyl-[2-(pyridin-3-ylmethoxy)-ethyl]-carbamoyl}-2-phenyl-ethyl)-N-(4-morpholin-4-yl-benzyl)-3-(4-trifluoromethyl-phenyl)-acrylamide;
(S)—N-[4-(4-Acetyl-piperazin-1-yl)-benzyl]-N-(1-{methyl-[2-(5-methyl-isoxazol-3-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(pyridin-4-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(5-trifluoromethyl-furan-2-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-cyclopropylmethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-(1-{methyl-[2-(pyridin-4-ylmethoxy)-ethyl]-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-(1-{methyl-[2-(5-methyl-isoxazol-3-ylmethoxy)-ethyl]-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-(1-{[2-(2-benzyloxy-ethoxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-cyanomethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-{1-[(2-allyloxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-{1-[(2-carbamoylmethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-(1-{methyl-[2-(pyridin-2-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(pyridin-2-ylmethoxy)-ethyl]-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-(1-{[2-(2-benzyloxy-ethoxy)-ethyl]-methyl-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-{1-[(2-cyanomethoxy-ethyl)-methyl-carbamoyl]-2-phenyl-ethyl}-3-(6-trifluoromethyl-pyridin-3-yl)-acrylamide;
(S)—N-[4-(4-Acetyl-piperazin-1-yl)-benzyl]-N-{1-[(2-allyloxy-ethyl)-methyl-carbamoyl]-2-phenyl-ethyl}-3-(6-trifluoromethyl-pyridin-3-yl)-acrylamide;
(S)—N-[1-({2-[(5-Bromo-furan-2-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-(1-{[2-(Benzyl-methyl-amino)-ethyl]-methyl-carbamoyl}-2-phenyl-ethyl)-N-(4-pyridin-2-yl-benzyl)-3-(4-trifluoromethyl-phenyl)-acrylamide;
(S)—N-[1-({2-[(6-Chloro-pyridin-3-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-(1-{[2-(Furan-3-ylmethyl-methyl-amino)-ethyl]-methyl-carbamoyl}-2-phenyl-ethyl)-N-(4-pyridin-2-yl-benzyl)-3-(4-trifluoromethyl-phenyl)-acrylamide;
(S)—N-(1-{[2-(Furan-2-ylmethyl-methyl-amino)-ethyl]-methyl-carbamoyl}-2-phenyl-ethyl)-N-(4-pyridin-2-yl-benzyl)-3-(4-trifluoromethyl-phenyl)-acrylamide;
(S)—N-(1-{Methyl-[2-(methyl-pyridin-2-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-(1-{Methyl-[2-(methyl-thiophen-2-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-[1-({2-[(5-Chloro-thiophen-2-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(6-Bromo-pyridin-3-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(5-Hydroxymethyl-furan-2-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(6-Methoxy-pyridin-3-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-(Methyl-{2-[methyl-(6-trifluoromethyl-pyridin-3-ylmethyl)-amino]-ethyl}-carbamoyl)-2-phenyl-ethyl]-N-(4-pyridin-2-yl-benzyl)-3-(4-trifluoromethyl-phenyl)-acrylamide;
(S)—N-(1-{Methyl-[2-(methyl-pyridin-3-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-(1-{Methyl-[2-(methyl-thiophen-3-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-[1-(Methyl-{2-[methyl-(2-methyl-benzyl)-amino]-ethyl}-carbamoyl)-2-phenyl-ethyl]-N-(4-pyridin-2-yl-benzyl)-3-(4-trifluoromethyl-phenyl)-acrylamide;
(S)—N-[1-({2-[(2,4-Dimethyl-benzyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(3,5-Dimethoxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[4-(4-Acetyl-piperazin-1-yl)-benzyl]-N-[1-({2-[(3,5-dimethoxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{[2-({4-[(2-hydroxy-ethyl)-methyl-amino]-benzyl}-methyl-amino)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-[1-({2-[(4-hydroxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(4-diethylamino-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(3-hydroxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{methyl-[2-(methyl-pyridin-3-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-{[3-(2-hydroxy-ethoxy)-benzyl]-methyl-amino}-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-[1-({2-[(3-cyano-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(4-isopropoxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-(methyl-{2-[methyl-(4-methyl-3,4-dihydro-2H-benzo[1,4]oxazin-7-ylmethyl)-amino]-ethyl}-carbamoyl)-2-phenyl-ethyl]-3-(4-trifluoromethyl-phenyl)-acrylamide;
(S)—N-[4-(4-Acetyl-piperazin-1-yl)-benzyl]-N-{1-[(2-{[4-(3-dimethylamino-propoxy)-benzyl]-methyl-amino}-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-[1-({2-[(6-methoxy-pyridin-3-ylmethyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{methyl-[2-(methyl-thiazol-2-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(benzo[1,3]dioxol-5-ylmethyl-methyl-amino)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(methyl-pyrimidin-5-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-[1-({2-[(2,3-difluoro-4-methyl-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(3H-imidazol-4-ylmethyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{methyl-[2-(methyl-pyridin-4-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(benzyl-methyl-amino)-ethyl]-methyl-carbamoyl}-2-phenyl-ethyl)-3-(4-trifluoromethyl-phenyl)-acrylamide;
(S)—N-(1-{[2-({4-[(2-Hydroxy-ethyl)-methyl-amino]-benzyl]-methyl-amino}-ethyl)-methyl-carbamoyl}-2-phenyl-ethyl)-N-(4-morpholin-4-yl-benzyl)-3-(4-trifluoromethyl-phenyl)-acrylamide;
(S)—N-[1-({2-[(4-Hydroxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(4-Diethylamino-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(3-Hydroxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-(1-{Methyl-[2-(methyl-pyridin-3-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-{1-[(2-{[3-(2-Hydroxy-ethoxy)-benzyl]methyl-amino}-ethyl)-methyl-carbamoyl]-2-phenyl-ethyl}-N-(4-morpholin-4-yl-benzyl)-3-(4-trifluoromethyl-phenyl)-acrylamide;
(S)—N-[1-({2-[(3-Cyano-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(4-Isopropoxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-(Methyl-{2-[methyl-(4-methyl-3,4-dihydro-2H-benzo[1,4]oxazin-7-ylmethyl)-amino]-ethyl}-carbamoyl)-2-phenyl-ethyl]-N-(4-morpholin-4-yl-benzyl)-3-(4-trifluoromethyl-phenyl)-acrylamide;
(S)—N-[1-({2-[(6-Methoxy-pyridin-3-ylmethyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-(1-{Methyl-[2-(methyl-thiazol-2-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-(1-{[2-(Benzo[1,3]dioxol-5-ylmethyl-methyl-amino)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{Methyl-[2-(methyl-pyrimidin-5-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-[1-({2-[(2,3-Difluoro-4-methyl-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(3H-Imidazol-4-ylmethyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-(1-{Methyl-[2-(methyl-pyridin-4-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-(1-{Methyl-[4-(2-oxo-pyrrolidin-1-yl)-benzyl]-carbamoyl}-2-phenyl-ethyl)-N-(4-morpholin-4-yl-benzyl)-3-(6-trifluoromethyl-pyridin-3-yl)-acrylamide;
(S)-Cyclopropanecarboxylic acid (4-{[methyl-(2-{(4-morpholin-4-yl-benzyl)-[3-(6-trifluoromethyl-pyridin-3-yl)-acryloyl]-amino}-3-phenyl-propionyl)-amino]-methyl}-phenyl)-amide;
(S)—N-(1-{Methyl-[4-(2-oxo-piperidin-1-yl)-benzyl]-carbamoyl}-2-phenyl-ethyl)-N-(4-morpholin-4-yl-benzyl)-3-(6-trifluoromethyl-pyridin-3-yl)-acrylamide;
(S)—N-(4-{[Methyl-(2-{(4-morpholin-4-yl-benzyl)-[3-(6-trifluoromethyl-pyridin-3-yl)-acryloyl]-amino}-3-phenyl-propionyl)-amino]-methyl}-phenyl)-benzamide;
(S)—N-(1-{[4-(Acetyl-phenyl-amino)-benzyl]-methyl-carbamoyl}-2-phenyl-ethyl)-N-(4-morpholin-4-yl-benzyl)-3-(6-trifluoromethyl-pyridin-3-yl)-acrylamide;
(S)—N-(1-{[4-(2-Methoxy-ethylamino)-benzyl]-methyl-carbamoyl}-2-phenyl-ethyl)-N-(4-morpholin-4-yl-benzyl)-3-(6-trifluoromethyl-pyridin-3-yl)-acrylamide;
N-{1-[(2-Methoxy-ethyl)-methyl-carbamoyl]-2-morpholin-4-yl-ethyl}-N-(4-pyridin-2-yl-benzyl)-3-(4-trifluoromethyl-phenyl)-acrylamide; and
N-{1-[(2-Methoxy-ethyl)-methyl-carbamoyl]-2-pyrrol-1-yl-ethyl}-N-(4-pyridin-4-yl-benzyl)-3-(4-trifluoromethyl-phenyl)-acrylamide.

The compounds of formula I and their pharmaceutically acceptable salts can be used as medicaments, e.g. in the form of pharmaceutical compositions for enteral or parenteral administration, and are suitable for the treatment and/or prevention of the diseases mentioned herein, such as especially malaria.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a galenical administration form together with suitable, non-toxic, inert, pharmaceutically acceptable solid or liquid carrier materials and, if desired, usual pharmaceutical adjuvants.

In one embodiment, the invention relates to a method for the treatment or prevention of the diseases mentioned herein, such as especially malaria, said method comprising administering to a subject a pharmaceutically active amount of a compound of formula I.

The compounds of formula I or the above-mentioned pharmaceutical compositions may also be used in combination with one or more other therapeutically useful substances e.g. with other antimalarials like quinolines (e.g. quinine, chloroquine, amodiaquine, mefloquine, primaquine, and tafenoquine), peroxide antimalarials (e.g. artemisinin, artemether, and artesunate), pyrimethamine-sulfadoxine antimalarials (e.g. Fansidar®), hydroxynaphtoquinones (e.g. atovaquone), acroline-type antimalarials (e.g. pyronaridine), and other antiprotozoal agents like ethylstibamine, hydroxystilbamidine, pentamidine, stilbamidine, quinapyramine, puromycine, propamidine, nifurtimox, melarsoprol, nimorazole, nifuroxime, aminitrozole and the like.

The present invention also relates to the use of a compound of formula I for the preparation of a pharmaceutical composition, optionally for use in combination with one or more other therapeutically useful substances such as those mentioned in the preceding paragraph, for the prevention and/or treatment of the diseases mentioned herein, such as especially malaria.

The compounds of the formula I of the present invention may be prepared according to the procedures described herein, especially as described in the experimental part.

In general, all chemical transformations can be performed according to well-known standard methodologies as described in the literature or as described in the procedures below.

### Preparation of Compounds of Formula I:

### Method A:

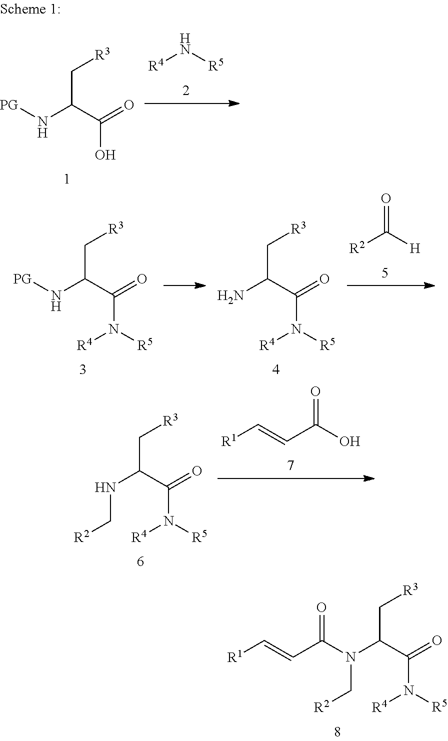

The Boc-protected amino-acid 1 can be coupled with an amine derivative 2 by the help of a coupling/activating reagent such as TBTU in a solvent such as DCM or DMF at rt in the presence of a base such as DIPEA (Hünig's base) to give the intermediate 3. Alternatively, the Cbz-protected amino-acid 1 can be coupled with the amine derivative 2 via the chloride intermediate (not depicted) generated by the help of a chlorinating agent such as the Ghosez's reagent in a solvent such as DCM at rt in the presence of a base such as TEA to give the intermediate 3. Boc-deprotection is usually achieved by reacting 3 with TFA in DCM, while Cbz-deprotection is achieved by hydrogenation with Pd/C catalyst in MeOH, to give the amine intermediate 4. Compound 4 can be refluxed with an aldehyde derivative 5 (under reductive amination conditions via the imine; not depicted) in MeOH in the presence of a base such as TEA to form an unstable imine intermediate which is reduced at rt with sodium borohydride to give the secondary amine intermediate 6. Alternatively, the reductive amination can be achieved in a solvent such as DCM in the presence of a reducing reagent such as sodium triacetoxyborohydride to give the expected secondary amine intermediate 6. Compound 6 can be acylated by either a carboxylic acid 7 by the help of a coupling/activating reagent such as TBTU or PyBop in a solvent such as DMF or MeCN at rt in the presence of a base such as DIPEA, or the corresponding acid chloride (not depicted) in a solvent such as DCM in the presence of a base such as TEA, to give the final compounds 8 of formula I.

The compounds of formula I can also be prepared via method B and according to Scheme 2.

### Method B:

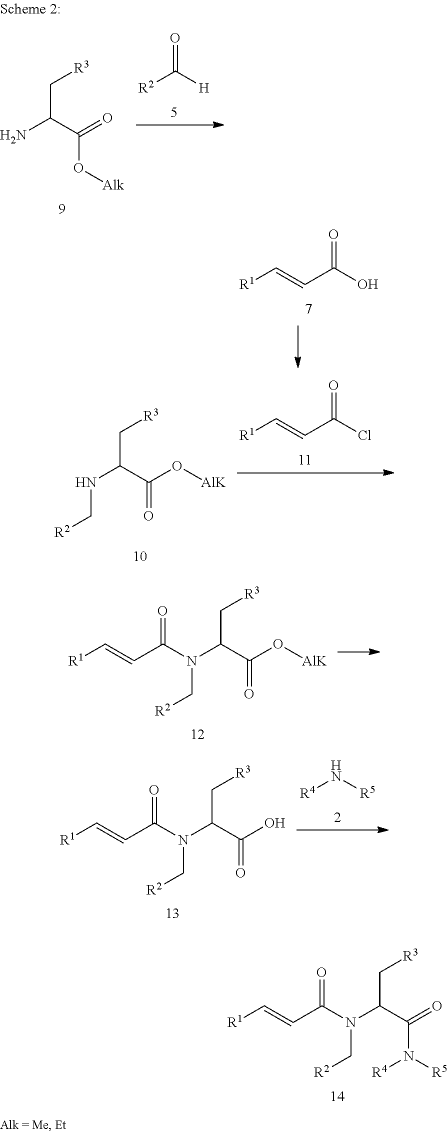

Reductive amination of an amino-acid methyl/ethyl ester 9 with an aldehyde derivative 5 either via the imine formation under conditions similar to those described above or in a solvent such as MeOH and in the presence of acetic acid and of a reducing reagent such as sodium cyanoborohydride gives the secondary amine intermediate 10. Compound 10 can be acylated by an acid chloride 11 in a solvent such as DCM in the presence of a base such as DIPEA or TEA to give the amide intermediate 12. The acid chloride can be generated by reaction of the corresponding carboxylic acid 7 either with oxalyl chloride in the presence of few drops of DMF or with the Ghosez's reagent, and in a solvent such as DCM.

Saponification of the ester function using methods known in the art such as treatment with a base such as NaOH in solvent mixtures such as methanol/water or ethanol/water followed by acylation of the resulting acid 13 with an amine derivative 2 with the help of a coupling/activating reagent such as TBTU or PyBrop in a solvent such as DCM in the presence of a base such as DIPEA provides the final compounds 14 of formula I.

The compounds of formula I wherein R⁵=—(CH₂)₂—O—R⁶ can also be prepared via method C and according to Scheme 3.

### Method C:

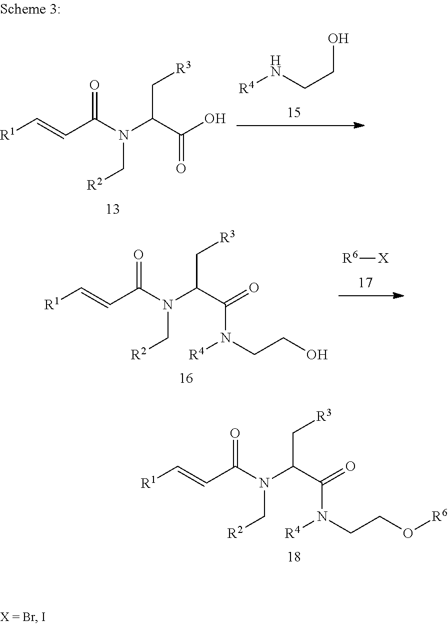

Coupling of the acid intermediate 13 with the aminoethanol derivative 15 under conditions similar to those described above followed by alkylation of the resulting hydroxyl intermediate 16 with a halide derivative 17 in the presence of a strong base such as sodium hydride and in a polar aprotic solvent such as THF provides the final compounds 18 of formula I.

The compounds of formula I wherein R⁴═R⁷, R⁵=—(CH₂)₂—NR⁷R⁸ and R⁸=alkyl, —CH₂-aryl or —CH₂-heteroaryl, can also be prepared via method D and according to Scheme 4.

### Method D:

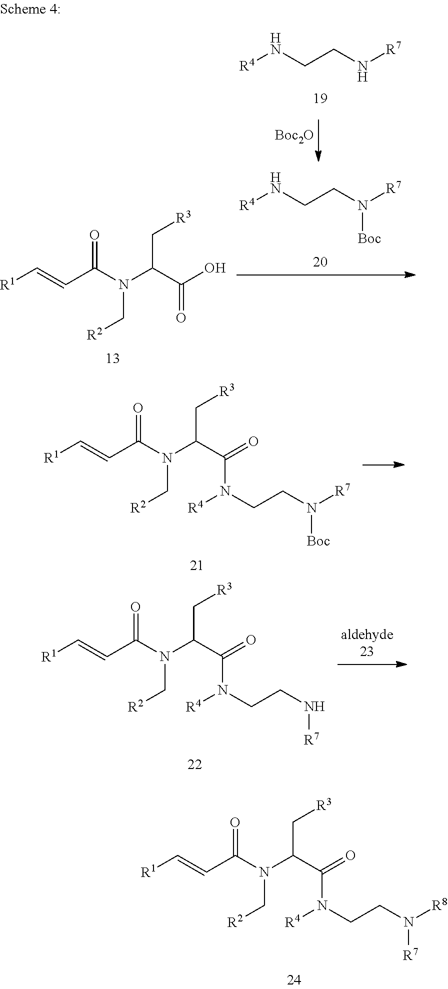

Coupling of the acid intermediate 13 with the Boc-protected ethylenediamine derivative 20 by the help of a coupling/activating reagent such as TBTU and a catalytic amount of DMAP in a solvent such as DCM at rt in the presence of a base such as DIPEA followed by Boc-deprotection of the amide intermediate 21 under conditions similar to those described above and then reductive amination of the resulting secondary amine 22 with an appropriate aldehyde derivative 23 in a solvent such as THF or MeCN in the presence of acetic acid and of a reducing reagent such as sodium triacetoxyborohydride provides the final compounds 24 of formula I.

The compounds of formula I wherein R⁵=—CH₂—(C₆H₄)—NR⁹R¹⁰ or —CH₂—(C₆H₄)—N(R¹¹)COR¹² can also be prepared via method E and according to Scheme 5.

### Method E:

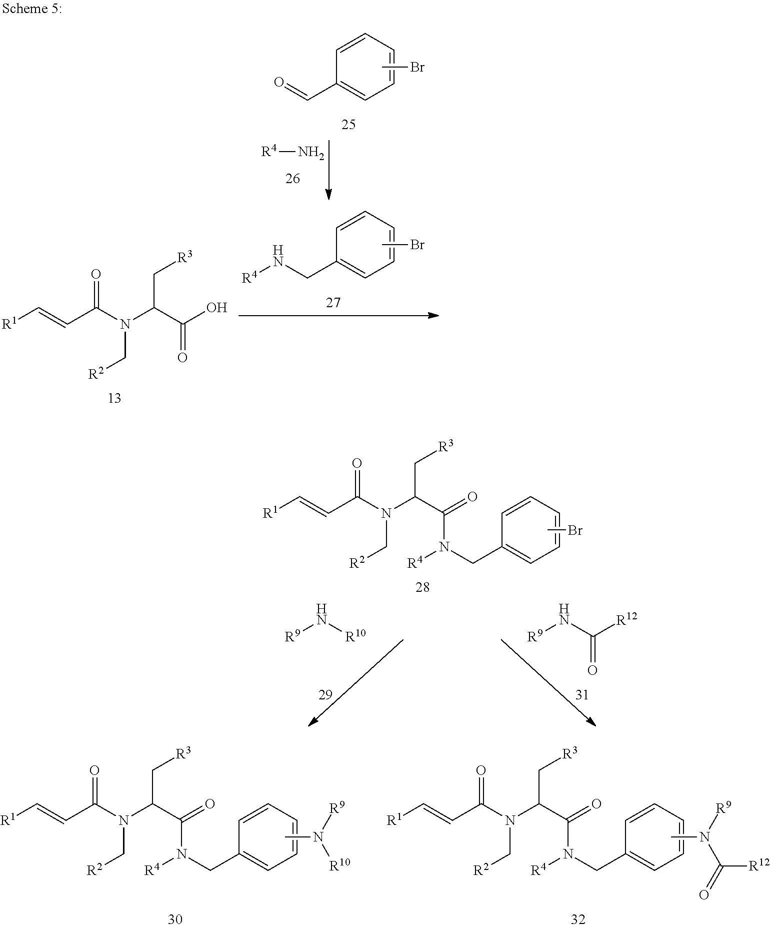

Coupling of the acid intermediate 13 with the amine 27 prepared via a reductive amination of 2-, 3-, or 4-bromobenzaldehyde 25 with a primary amine 26, under conditions similar to those described above, followed by Buchwald-Hartwig coupling of the aryl bromide intermediate 28 with an amine derivative 29, by the help of a catalyst such as SK-CC02-A in the presence of a base such as sodium tert-butoxide in a solvent such as dioxane, provides the final compounds 30 of formula I. In addition, aryl amidation of the aryl bromide intermediate 28 with an amide derivative 31, by the help of a catalyst such as copper (I) iodide in the presence of a ligand such as N,N′-dimethylethylenediamine and an inorganic base such as potassium carbonate in a solvent such as dioxane, provides the final compounds 32 of formula I.

The compounds of formula I wherein R³=—NR¹³R¹⁴ can also be prepared via method F and according to Scheme 6.

### Method F:

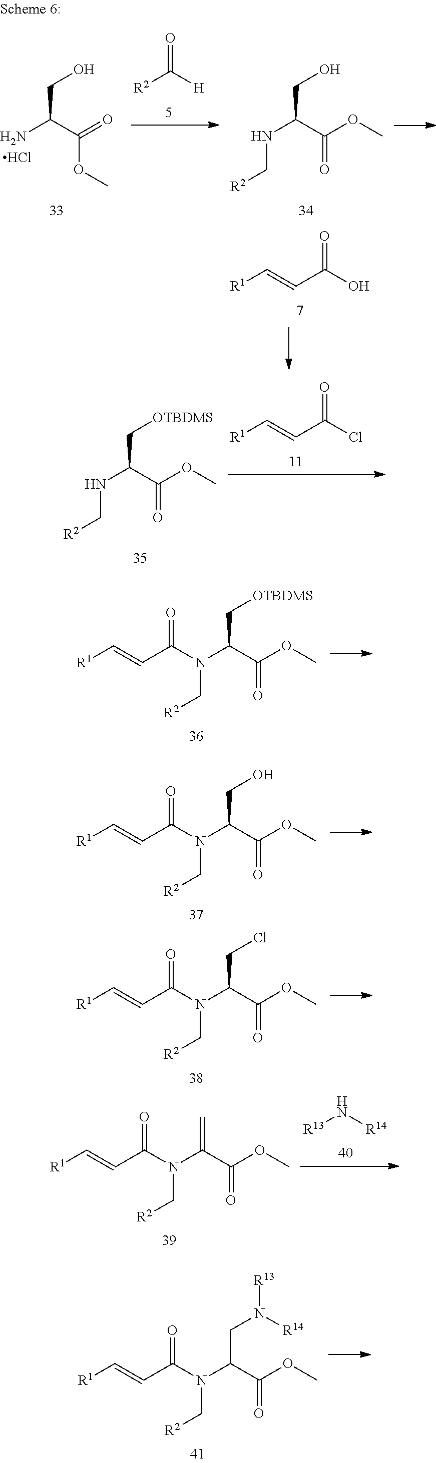

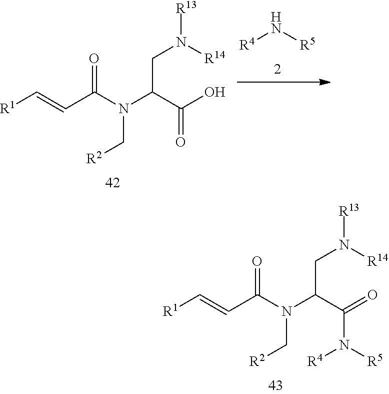

L-serine methyl ester 33 can be refluxed with an aldehyde derivative 5 (under reductive amination conditions via the imine; not depicted) in DCM in the presence of a base such as TEA and a dessicant such as sodium sulfate to form an unstable imine intermediate which is reduced at 0° C. in MeOH with sodium borohydride to give the secondary amine intermediate 34. Protection of the hydroxy group by tert-butyldimethylsilyl chloride in the presence of a catalyst such as imidazole in a solvent such as DCM gives the protected serine derivative 35. Compound 35 can be acylated by an acid chloride 11 in a solvent such as DCM in the presence of a base such as TEA and a catalytic amount of DMAP to give the amide intermediate 36. The acid chloride 11 can be generated by reaction of the corresponding carboxylic acid 7 with oxalyl chloride in the presence of few drops of DMF and in a solvent such as DCM.

TBDMS-deprotection is usually achieved by treating 36 in a solvent mixture such as acetic acid/water to give the alcohol intermediate 37. Chlorination of the hydroxy group of 37 with a chlorinating agent such as thionyl chloride in a solvent such as DCM gives the chloride intermediate 38. The elimination product 39 can be obtained by the use of a base such as TEA in a solvent such as DCM.

Conjugate addition on the double bond of compound 39 with an aliphatic cyclic secondary amine 40 in the presence of a catalyst such as FeCl₃ in a solvent such as DCM, or aza-Michael addition with an aromatic amine or a carbamate or an oxo-amide 40 in the presence of a base such as potassium carbonate in a solvent such as MeCN, gives the non-natural amino-acid derivative 41.

Saponification of the ester function using methods known in the art such as treatment with a base such as NaOH in solvent mixtures such as methanol/water followed by acylation of the resulting acid 42 with an amine derivative 2 with the help of a coupling/activating reagent such as TBTU in a solvent such as DCM in the presence of a base such as DIPEA provides the final compounds 43 of formula I.

Carboxylic acid compounds 7 are commercially available or can be synthesized according to the following pathways:

### Pathway A: Knoevenagel Reaction

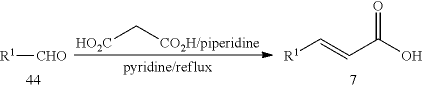

### Pathway B: Horner-Emmons Reaction

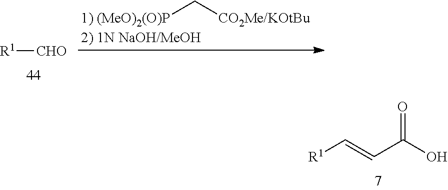

### Pathway C: Heck Reaction

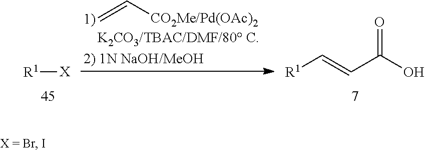

Pathway A: By reaction of an aldehyde 44 with malonic acid in the presence of a strong base such as piperidine in refluxing pyridine furnishes the desired carboxylic acid 7 (WO 00/66566).

Pathway B: By reaction of an aldehyde 44 with trimethyl phosphoacetate in the presence of a strong base such as KOtBu in an aprotic solvent such as THF followed by saponification of the resulting methyl ester with 1N NaOH in MeOH furnishes the desired carboxylic acid 7.

Pathway C: By reaction of a halide 45 with methyl acrylate in the presence of a base such as potassium carbonate, a palladium catalyst such as palladium (II) acetate and a phase-transfer catalyst TBAC in DMF followed by saponification of the resulting methyl ester with 1N NaOH in MeOH provides the desired carboxylic acid 7 (EP 0 702 014 A1).

Non-natural amino-acid derivatives 9 used in method B can be synthesized according to the following pathways:

### Pathway D: Paal-Knorr Pyrrole Synthesis

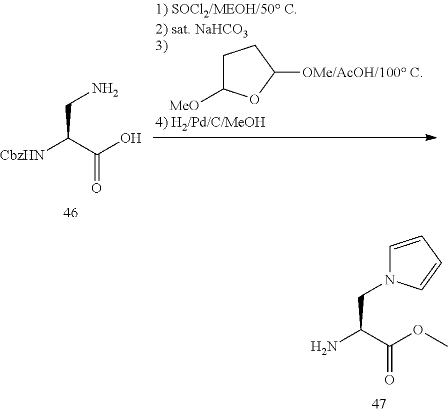

### Pathway E: Horner-Emmons Reaction

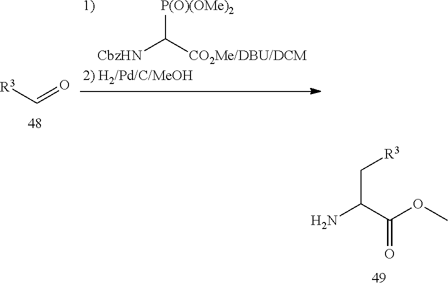

### Pathway F: Nucleophilic Substitution

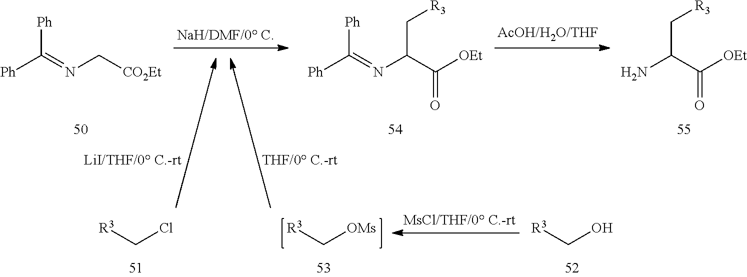

Pathway D: By reaction of the free amine Cbz-L-2,3-diaminopropionic acid methyl ester, prepared from the acid 46 by methylation (Helv. Chim. Acta 1989, 72, 1043-51), with 2,5-dimethoxytetrahydrofuran in AcOH at 100° C. (Acta Chem. Scand. 1952, 6, 867-74), followed by Cbz-deprotection of the resulting protected pyrrole amino-acid by hydrogenation with Pd/C catalyst in MeOH furnishes the methyl ester pyrrole amino-acid 47.

Pathway E: By reaction of an aldehyde 48 with (+/−)-Cbz-α-phosphonoglycine trimethyl ester in the presence of a strong base such as DBU in an aprotic solvent such as DCM, followed by reduction of the resulting double bond and Cbz-deprotection (one pot) by hydrogenation with Pd/C catalyst in MeOH furnishes the desired methyl ester amino-acid 49 (WO 2007/070826).

Pathway F: By reaction of a chloride 51 in the presence of lithium iodide or a mesylate 53 generated from an alcohol 52 (with mesyl chloride in an aprotic solvent such as THF) with the anion of N-(diphenylmethylene)-glycine ethyl ester 50 in a DMF/THF mixture, followed by deprotection of the resulting imine-protected amino-acid 54 in an AcOH/H₂O/THF mixture provides the desired ethyl ester amino-acid 55 (WO 2006/045613, WO 2005/016883, WO 01/68591).

The following examples illustrate the invention but do not limit the scope thereof. All temperatures are stated in ° C.

### Abbreviations (as Used Herein):

AcOH acetic acid
Alk alkyl
aq. aqueous
Boc tert.-butyloxycarbonyl
Boc₂O di-tert-butyldicarbonate
cat. catalytic
Cbz benzyloxycarbonyl
conc. concentrated
DAD diode array detection
DBU 1,8-diazabicyclo[5.4.0]undec-7-ene
DCM dichloromethane
DIPEA N,N-diisopropylethylamine
DMAP N,N-dimethyl-4-aminopyridine
DMF dimethylformamide
DMSO dimethylsulfoxide
EA ethyl acetate
ELSD evaporative light scattering detection
eq equivalent(s)
ESI electrospray ionization
Et ethyl
EtOH ethanol
Ex. example
FC flash chromatography
h hour(s)
HPLC high performance liquid chromatography
KOtBu potassium tert-butoxide
LC-MS liquid chromatography-mass spectroscopy
Me methyl
MeCN acetonitrile
MeOH methanol
min minute(s)
MS mass spectroscopy
Ms mesyl
MsCl mesyl chloride
No. number
OAc acetate
PBS phosphate buffered saline
PG protecting group
Ph phenyl
PyBop benzotriazol-1-yl-oxy-tris-pyrrolidinophosphonium hexafluorophosphate
PyBrop bromo-tris-pyrrolidinophosphonium hexafluorophosphate
quant. quantitative
rt room temperature
sat. saturated
SK-CC02-A 2-(dimethylamino)-ferrocen-1-yl-palladium(11)-chloride dinorbornylphosphine complex (Fluke 44696)
TBAC tetra-n-butylammonium chloride
TBDMS tert-butyldimethylsilyl
TBDMSCl tert-butyldimethylsilyl chloride
TBTU O-benzotriazol-1-yl-N,N,N′,N′-tetramethyluronium tetrafluoroborate
TEA triethylamine
TFA trifluoroacetic acid
THF tetrahydrofuran
t_{R} retention time
UV ultra violet
V is visible

### General Procedures and Examples:

### HPLC Conditions:

### Analytic:

(A) Agilent 1100 series with UV/Vis and MS detection (MS: Thermo Finnigan single quadrupole). Columns (4.6×50 mm, 5 μm): Zorbax SB-AQ, Zorbax Extend C18 or Waters X-Bridge C18. Acidic conditions: eluents: A: MeCN, B: H₂O+0.04% TFA. Basic conditions: eluents: A: MeCN, B: conc. NH₃ in water (1.0 mL/L). Gradient 5 to 95% A over 1.5 min. Flow rate: 4.5 mL/min.

(B) Agilent 1100 series with DAD, ELSD and MS detection (MS: ESI⁺/ESI⁻, AB Sciex Instruments API 2000 triple quadrupole). Column: Onyx monolithic C18 (100×3 mm). Conditions: eluents: A: MeCN, B: H₂O+0.05% formic acid. Gradient 10 to 90% A over 4.0 min. Flow rate: 1.8 mL/min.

### Preparative:

Gilson with UV/Vis+MS or UV/Vis+ELSD detection. Acidic conditions: eluents: A: MeCN, B: H₂O+0.5% formic acid. Basic conditions: eluents: A: MeCN, B: H₂O+0.5% NH₃ (25% aq.).

(A) Waters X-Bridge column, 19×50 mm, 5 μm. Gradient: 20 to 90% A over 5 min. Flow rate: 40 mL/min.

(B) Waters X-Bridge column, 30×75 mm, 10 μm. Gradient: 20 to 90% A over 6 min. Flow rate: 75 mL/min.

### Preparation of Compounds of Formula I Via Method A:

### Step 1

### General Procedure 1

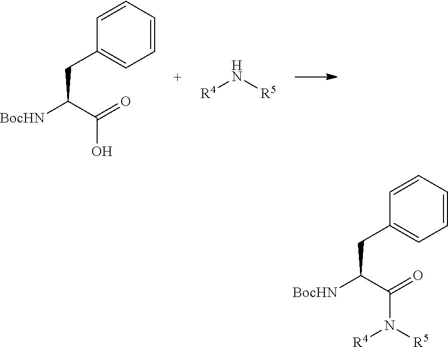

To a solution of the acid Boc-L-phenylalanine (1 eq) in dry DCM or DMF (1 mL/mmol) were added TBTU (1 eq) and DIPEA (5 eq). The resulting white suspension was stirred at rt for 30 min, then a solution of the amine NHR⁴R⁵ (1 eq) in dry DCM or DMF (0.5 mL/mmol) was added. The reaction mixture was stirred at rt overnight under nitrogen atmosphere, then concentrated in vacuo. The resulting residue was diluted in EA. The organic layer was washed with water, sat. NaHCO₃ solution and brine, dried (MgSO₄), filtered and concentrated under reduced pressure. FC (n-heptane/EA system) afforded the pure amide.

| 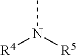 | Chemical name | Yield | LC-MS* t_{R} (min) [M + H]⁺ | |
|---|---|---|---|---|
| 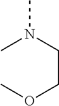 | (S)-{1-[(2-Methoxy-ethyl)-methyl- carbamoyl]-2-phenyl-ethyl}-carbamic acid tert-butyl ester | 97% | 0.96 | 337.48 |
| 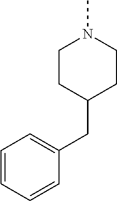 | (S)-[1-Benzyl-2-(4-benzyl-piperidin-1- yl)-2-oxo-ethyl]-carbamic acid tert- butyl ester | 98% | 1.09 | 423.24 |
| 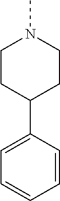 | (S)-[1-Benzyl-2-oxo-2-(4-phenyl- piperidin-1-yl)-ethyl]-carbamic acid tert-butyl ester | 94% | 1.07 | 409.21 |
| 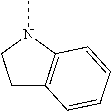 | (S)-[1-Benzyl-2-(2,3-dihydro-indol-1- yl)-2-oxo-ethyl]-carbamic acid tert- butyl ester | 80% | 1.04 | 367.14 |
| 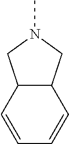 | (S)-[1-Benzyl-2-oxo-2-(1,3,3a,7a- tetrahydro-isoindol-2-yl)-ethyl]- carbamic acid tert-butyl ester | 61% | 1.00 | 367.15 |

*Analytic A, Zorbax SB-AQ column, acidic conditions

### General Procedure 2

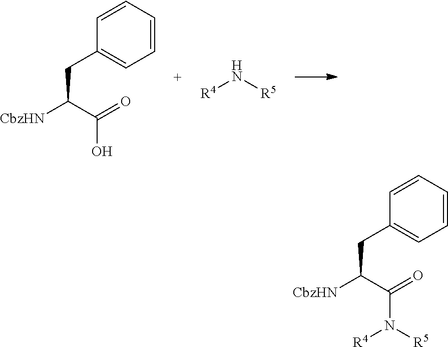

To an ice-cooled solution of the acid Cbz-L-phenylalanine (1 eq) in dry DCM (2.5 mL/mmol) was added 1-chloro-N,N-2-trimethylpropenylamine (Ghosez's reagent, 1 eq). The resulting mixture was stirred at 0° C. for 10 min, then the amine NHR⁴R⁵ (1 eq) and TEA (1 eq) were added. The reaction mixture was stirred at rt overnight, then diluted with DCM, washed with a sat. NaHCO₃ solution, dried (MgSO₄), filtered and concentrated under reduced pressure. FC (DCM/MeOH system) afforded the pure amide.

| 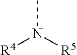 | Chemical name | Yield | LC-MS* t_{R} (min) [M + H]⁺ | |
|---|---|---|---|---|
| 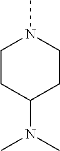 | (S)-[1-Benzyl-2-(4-dimethylamino- piperidin-1-yl)-2-oxo-ethyl]-carbamic acid benzyl ester | crude | 0.69 | 410.12 |
| 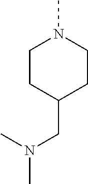 | (S)-[1-Benzyl-2-(4- dimethylaminomethyl-piperidin-1-yl)-2- oxo-ethyl]-carbamic acid benzyl ester | crude | 0.74 | 424.24 |
| 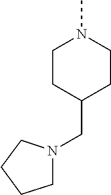 | (S)-[1-Benzyl-2-oxo-2-(4-pyrrolidin-1- ylmethyl-piperidin-1-yl)-ethyl]- carbamic acid benzyl ester | 32% | 0.77 | 450.12 |
| 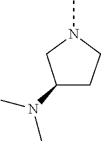 | (S,R)-[1-Benzyl-2-(3-dimethylamino- pyrrolidin-1-yl)-2-oxo-ethyl]-carbamic acid benzyl ester | 79% | 0.69 | 396.16 |
| 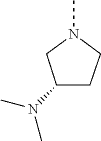 | (S,S)-[1-Benzyl-2-(3-dimethylamino- pyrrolidin-1-yl)-2-oxo-ethyl]-carbamic acid benzyl ester | 78% | 0.71 | 396.16 |

*Analytic A, Zorbax SB-AQ column, acidic conditions

### Step 2

### General Procedure 1

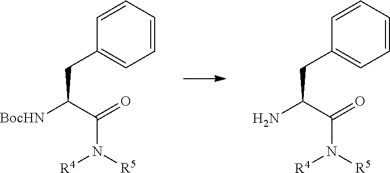

To an ice-cooled solution of the Boc-protected amine (1 eq) in dry DCM (15 mL/mmol) was added dropwise TFA (10 eq). The resulting reaction mixture was stirred at rt for 2 h under nitrogen atmosphere and then concentrated in vacuo. The resulting residue was dissolved in DCM, washed with a sat. NaHCO₃ solution, and the aq. phase was extracted twice with DCM. The combined organic extracts were washed with brine, dried (MgSO₄), filtered and concentrated under reduced pressure to afford the free primary amine, which was used for the next step without further purification.

| 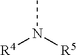 | Chemical name | Yield | LC-MS* t_{R} (min) [M + H]⁺ | |
|---|---|---|---|---|
| 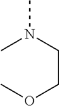 | (S)-2-Amino-N-(2-methoxy-ethyl)-N- methyl-3-phenyl-propionamide | 83% | 0.59 | 237.18 |
| 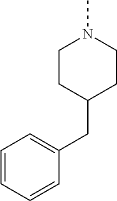 | (S)-2-Amino-1-(4-benzyl-piperidin-1- yl)-3-phenyl-propan-1-one | 95% | 0.81 | 323.18 |
| 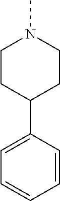 | (S)-2-Amino-3-phenyl-1-(4-phenyl- piperidin-1-yl)-propan-1-one | 87% | 0.75 | 309.14 |
| 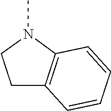 | (S)-2-Amino-1-(2,3-dihydro-indol-1-yl)- 3-phenyl-propan-1-one | 98% | 0.73 | 267.09 |
| 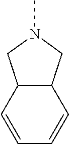 | (S)-2-Amino-3-phenyl-1-(1,3,3a,7a- tetrahydro-isoindol-2-yl)-propan-1-one | 86% | 0.71 | 267.08 |

*Analytic A, Zorbax SB-AQ column, acidic conditions

### General Procedure 2

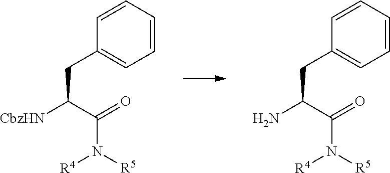

To a purged solution of the Cbz-protected amine (1 eq) in dry MeOH (2.5 mL/mmol) was added 10% Pd/C (10% w/w) under nitrogen atmosphere. The flask was evacuated and refilled with hydrogen (3×). The black suspension was stirred at rt overnight under hydrogen atmosphere, then filtered over Celite and concentrated under reduced pressure to afford the crude primary amine, which was used for the next step without further purification.

| 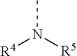 | Chemical name | LC-MS* t_{R} (min) [M + H]⁺ | |
|---|---|---|---|
| 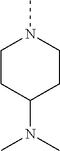 | (S)-2-Amino-1-(4- dimethylamino-piperidin-1- yl)-3-phenyl-propan-1-one | 0.33 | 276.46 |
| 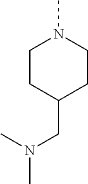 | (S)-2-Amino-1-(4- dimethylaminomethyl- piperidin-1-yl)-3-phenyl- propan-1-one | 0.42 | 290.16 |
| 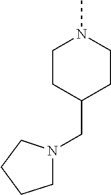 | (S)-2-Amino-3-phenyl- 1-(4-pyrrolidin-1-ylmethyl- piperidin-1-yl)-propan-1-one | 0.49 | 316.20 |
| 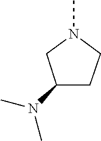 | (S,R)-2-Amino-1-(3- dimethylamino-pyrrolidin-1- yl)-3-phenyl-propan-1-one | 0.28 | 262.11 |
| 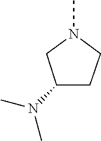 | (S,S)-2-Amino-1-(3- dimethylamino-pyrrolidin-1- yl)-3-phenyl-propan-1-one | 0.28 | 262.12 |

*Analytic A, Zorbax SB-AQ column, acidic conditions

### Step 3

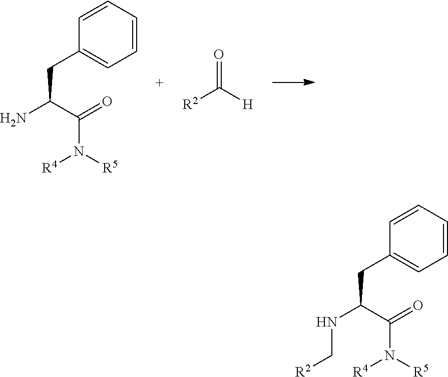

### General Procedure 1

To a solution of the amine (1 eq) in dry MeOH (4 mL/mmol) was added the aldehyde R²CHO (1 eq). The resulting mixture was refluxed overnight under nitrogen atmosphere. After cooling to 0° C., sodium borohydride (2 eq) was added portionwise. The reaction mixture was stirred at rt for 1 h, then quenched with a sat. NaHCO₃ solution and extracted twice with EA. The combined organic extracts were washed with brine, dried (MgSO₄), filtered and concentrated under reduced pressure. FC (EA, EA/MeOH, DCM/MeOH or DCM/MeOH+1% NH₄OH system) afforded the pure secondary amine.

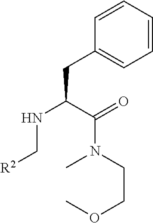

| | | | LC-MS* | |
|---|---|---|---|---|
| R² | Chemical name | Yield | t_{R} (min) | [M + H]⁺ |
| 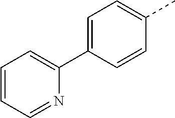 | (S)-N-(2-Methoxy-ethyl)-N-methyl-3- phenyl-2-(4-pyridin-2-yl- benzylamino)-propionamide | 83% | 0.66 | 404.27 |
| 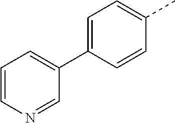 | (S)-N-(2-Methoxy-ethyl)-N-methyl-3- phenyl-2-(4-pyridin-3-yl- benzylamino)-propionamide | 62% | 0.63 | 404.08 |
| 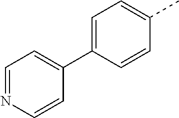 | (S)-N-(2-Methoxy-ethyl)-N-methyl-3- phenyl-2-(4-pyridin-4-yl- benzylamino)-propionamide | 41% | 0.61 | 404.09 |
| 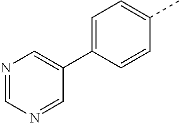 | (S)-N-(2-Methoxy-ethyl)-N-methyl-3- phenyl-2-(4-pyrimidin-5-yl- benzylamino)-propionamide | 89% | 0.72 | 405.09 |
| 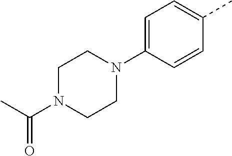 | (S)-2-[4-(4-Acetyl-piperazin-1-yl)- benzylamino]-N-(2-methoxy-ethyl)- N-methyl-3-phenyl-propionamide | 91% | 0.77 | 453.77 |
| 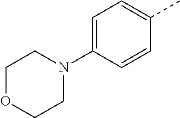 | (S)-N-(2-Methoxy-ethyl)-N-methyl-2- (4-morpholin-4-yl-benzylamino)-3- phenyl-propionamide | 82% | 0.76 | 412.61 |

*Analytic A, Zorbax SB-AQ column, acidic conditions

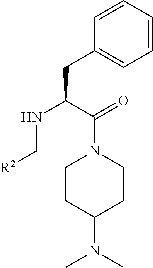

| | | | LC-MS* | |
|---|---|---|---|---|
| R² | Chemical name | Yield | t_{R} (min) | [M + H]⁺ |
| 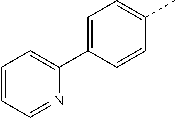 | (S)-1-(4-Dimethylamino-piperidin-1- yl)-3-phenyl-2-(4-pyridin-2-yl- benzylamino)-propan-1-one | 87% | 0.51 | 442.93 |
| 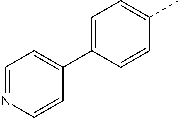 | (S)-1-(4-Dimethylamino-piperidin-1- yl)-3-phenyl-2-(4-pyridin-4-yl- benzylamino)-propan-1-one | 79% | 0.49 | 442.87 |
| 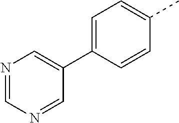 | (S)-1-(4-Dimethylamino-piperidin-1- yl)-3-phenyl-2-(4-pyrimidin-5-yl- benzylamino)-propan-1-one | 95% | 0.57 | 444.12 |
| 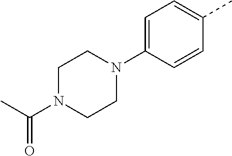 | (S)-2-[4-(4-Acetyl-piperazin-1-yl)- benzylamino]-1-(4-dimethylamino- piperidin-1-yl)-3-phenyl-propan-1- one | 92% | 0.56 | 492.17 |
| 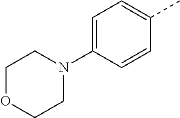 | (S)-1-(4-Dimethylamino-piperidin-1- yl)-2-(4-morpholin-4-yl- benzylamino)-3-phenyl-propan-1- one | 62% | 0.60 | 451.66 |

*Analytic A, Zorbax SB-AQ column, acidic conditions

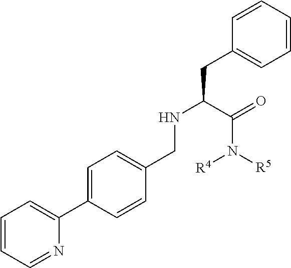

| | | | LC-MS* | |
|---|---|---|---|---|
| 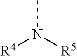 | Chemical name | Yield | t_{R} (min) | [M + H]⁺ |
| 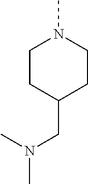 | (S)-1-(4-Dimethyl- aminomethyl- piperidin-1-yl)-3- phenyl- 2-(4-pyridin-2- yl-benzylamino)- propan-1-one | 53% | 0.54 | 457.30 |
| 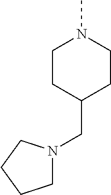 | (S)-3-Phenyl-2- (4-pyridin-2-yl- benzylamino)-1- (4-pyrrolidin-1- ylmethyl-piperidin- 1-yl)-propan-1-one | 49% | 0.56 | 483.21 |
| 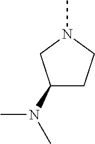 | (S,R)-1-(3-Dimethyl- amino-pyrrolidin- 1-yl)-3-phenyl-2- (4-pyridin-2-yl- benzylamino)- propan-1-one | 86% | 0.52 | 429.23 |
| 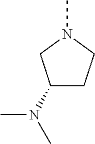 | (S,S)-1-(3-Dimethyl- amino-pyrrolidin- 1-yl)-3-phenyl-2- (4-pyridin-2-yl- benzylamino)- propan-1-one | 99% | 0.53 | 429.22 |

*Analytic A, Zorbax SB-AQ column, acidic conditions

### General Procedure 2

To a solution of the amine (1 eq) in dry DCM (20 mL/mmol) were added successively the aldehyde R²CHO (1 eq) and sodium triacetoxyborohydride (3 eq). The reaction mixture was stirred at rt overnight under nitrogen atmosphere, then quenched with a sat. NH₄Cl solution and extracted twice with EA. The combined organic extracts were washed with brine, dried (MgSO₄), filtered and concentrated under reduced pressure to afford the crude secondary amine.

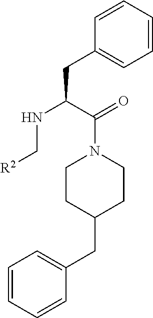

| | | | LC-MS* | |
|---|---|---|---|---|
| | Chemical | | t_{R} | |
| R² | name | Yield | (min) | [M + H]⁺ |
| 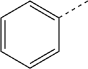 | (S)-2- Benzylamino- 1-(4-benzyl- piperidin-1-yl)- 3-phenyl- propan-1- one | quant. | 0.91 | 413.24 |
| 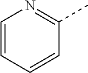 | (S)-1-(4-Benzyl- piperidin-1-yl)- 3-phenyl-2- [(pyridin- 2-ylmethyl)- amino]- propan- 1-one | quant. | 0.87 | 414.24 |
| 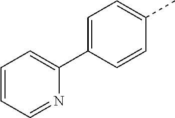 | (S)-1-(4- Benzyl- piperidin- 1-yl)-3- phenyl-2-(4- pyridin-2-yl- benzylamino)- propan-1-one | 81% | 0.84 | 490.24 |
| 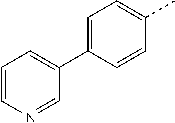 | (S)-1-(4- Benzyl- piperidin-1- yl)-3- phenyl-2-(4- pyridin-3-yl- benzylamino)- propan-1-one | 87% | 0.80 | 490.26 |

*Analytic A, Zorbax SB-AQ column, acidic conditions

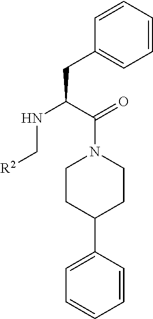

| | | | LC-MS* | |
|---|---|---|---|---|
| | Chemical | | t_{R} | |
| R² | name | Yield | (min) | [M + H]⁺ |
| 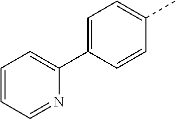 | (S)-3-Phenyl-1- (4-phenyl- piperidin-1-yl)- 2-(4-pyridin-2- yl-benzylamino)- propan-1-one | 95% | 0.85 | 476.27 |
| 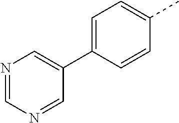 | (S)-3-Phenyl-1- (4-phenyl- piperidin-1- yl)-2-(4- pyrimidin-5- yl- benzylamino)- propan-1-one | 95% | 0.89 | 477.26 |

*Analytic A, Zorbax SB-AQ column, acidic conditions

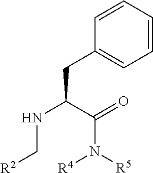

| | | | | LC-MS* | |
|---|---|---|---|---|---|
| R² | 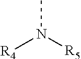 | Chemical name | Yield | t_{R} (min) | [M + H]⁺ |
| 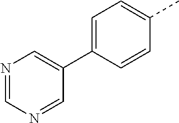 | 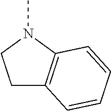 | (S)-1-(2,3-Dihydro-indol-1-yl)- 3-phenyl-2-(4-pyrimidin-5-yl- benzylamino)-propan-1-one | quant. | 0.81 | 435.12 |
| 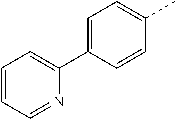 | 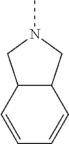 | (S)-3-Phenyl-2-(4-pyridin-2- yl-benzylamino)-1-(1,3,3a,7a- tetrahydro-isoindol-2-yl)- propan-1-one | 43% | 0.73 | 434.14 |

*Analytic A, Zorbax SB-AQ column, acidic conditions

### Step 4

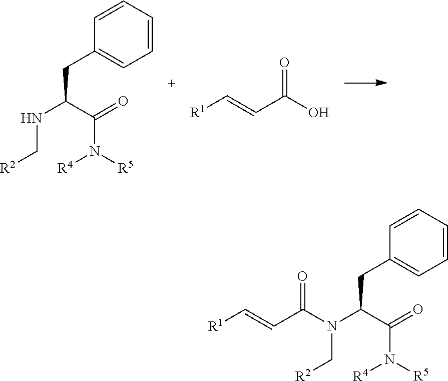

### General Procedure 1

To the cinnamic acid (1 eq) were added successively a solution of TBTU or PyBop (1 eq) in dry MeCN or DMF (5 mL/mmol), and DIPEA (5 eq). The resulting mixture was stirred at rt for 30 min and then a solution of the amine (1 eq) in dry MeCN or DMF (5 mL/mmol) was added. The reaction mixture was stirred at rt or at 60° C. overnight under nitrogen atmosphere, then directly purified by preparative HPLC to afford the pure final compound.

### General Procedure 2

To an ice-cooled solution of the cinnamic acid (1 eq) in dry DCM (30 mL/mmol) was added 1-chloro-N,N-2-trimethylpropenylamine (Ghosez's reagent, 1 eq). The resulting mixture was stirred at 0° C. for 10 min, then the amine (1 eq) and TEA (1.1 eq) were added. The reaction mixture was stirred at rt overnight, then diluted with DCM, washed with a sat. NaHCO₃ solution, dried (MgSO₄), filtered and concentrated under reduced pressure. The crude product was purified by preparative HPLC to afford the pure final compound.

| Example | | LC-MS* | |
|---|---|---|---|
| number | Chemical name | t_{R} (min) | [M + H]⁺ |
| 1 | (S)-N-[1-Benzyl-2-(4-dimethylamino-piperidin-1-yl)-2- | 0.80 | 613.19 |
| | oxo-ethyl]-N-(4-morpholin-4-yl-benzyl)-3-(1,3,5- | | |
| | trimethyl-1H-pyrazol-4-yl)-acrylamide | | |
| 2 | (S)-N-[1-Benzyl-2-(4-dimethylamino-piperidin-1-yl)-2- | 0.82 | 616.08 |
| | oxo-ethyl]-3-(2,4-dimethyl-thiazol-5-yl)-N-(4-morpholin- | | |
| | 4-yl-benzyl)-acrylamide | | |
| 3 | (S)-N-[1-Benzyl-2-(4-dimethylamino-piperidin-1-yl)-2- | 0.81 | 600.24 |
| | oxo-ethyl]-3-(3,5-dimethyl-isoxazol-4-yl)-N-(4- | | |
| | morpholin-4-yl-benzyl)-acrylamide | | |
| 4 | (S)-N-[1-Benzyl-2-(4-dimethylamino-piperidin-1-yl)-2- | 0.87 | 650.77 |
| | oxo-ethyl]-N-(4-morpholin-4-yl-benzyl)-3-(5- | | |
| | trifluoromethyl-pyridin-2-yl)-acrylamide | | |
| 5 | (S)-N-[1-Benzyl-2-(4-dimethylamino-piperidin-1-yl)-2- | 0.82 | 612.21 |
| | oxo-ethyl]-3-(6-methoxy-pyridin-3-yl)-N-(4-morpholin-4- | | |
| | yl-benzyl)-acrylamide | | |
| 6 | (S)-N-[1-Benzyl-2-(4-dimethylamino-piperidin-1-yl)-2- | 0.81 | 641.32 |
| | oxo-ethyl]-N-(4-pyridin-2-yl-benzyl)-3-(4-trifluoromethyl- | | |
| | phenyl)-acrylamide | | |
| 7 | (S)-N-[1-Benzyl-2-(4-dimethylamino-piperidin-1-yl)-2- | 0.77 | 642.18 |
| | oxo-ethyl]-N-(4-pyridin-2-yl-benzyl)-3-(6-trifluoromethyl- | | |
| | pyridin-3-yl)-acrylamide | | |
| 8 | (S)-N-[1-Benzyl-2-(4-dimethylamino-piperidin-1-yl)-2- | 0.75 | 591.54 |
| | oxo-ethyl]-3-(2,5-dimethyl-2H-pyrazol-3-yl)-N-(4-pyridin- | | |
| | 2-yl-benzyl)-acrylamide | | |
| 9 | (S)-N-[1-Benzyl-2-(4-dimethylamino-piperidin-1-yl)-2- | 0.75 | 605.18 |
| | oxo-ethyl]-N-(4-pyridin-2-yl-benzyl)-3-(1,3,5-trimethyl- | | |
| | 1H-pyrazol-4-yl)-acrylamide | | |
| 10 | (S)-N-[1-Benzyl-2-(4-dimethylamino-piperidin-1-yl)-2- | 0.76 | 608.02 |
| | oxo-ethyl]-3-(2,4-dimethyl-thiazol-5-yl)-N-(4-pyridin-2-yl- | | |
| | benzyl)-acrylamide | | |
| 11 | (S)-N-[1-Benzyl-2-(4-dimethylamino-piperidin-1-yl)-2- | 0.74 | 593.89 |
| | oxo-ethyl]-3-(2-methyl-thiazol-4-yl)-N-(4-pyridin-2-yl- | | |
| | benzyl)-acrylamide | | |
| 12 | (S)-N-[1-Benzyl-2-(4-dimethylamino-piperidin-1-yl)-2- | 0.76 | 592.33 |
| | oxo-ethyl]-3-(3,5-dimethyl-isoxazol-4-yl)-N-(4-pyridin-2- | | |
| | yl-benzyl)-acrylamide | | |
| 13 | (S)-N-[1-Benzyl-2-(4-dimethylamino-piperidin-1-yl)-2- | 0.74 | 592.13 |
| | oxo-ethyl]-3-(2,5-dimethyl-oxazol-4-yl)-N-(4-pyridin-2-yl- | | |
| | benzyl)-acrylamide | | |
| 14 | (S)-N-[1-Benzyl-2-(4-dimethylamino-piperidin-1-yl)-2- | 0.77 | 608.13 |
| | oxo-ethyl]-3-(6-chloro-pyridin-3-yl)-N-(4-pyridin-2-yl- | | |
| | benzyl)-acrylamide | | |
| 15 | (S)-N-[1-Benzyl-2-(4-dimethylamino-piperidin-1-yl)-2- | 0.71 | 588.32 |
| | oxo-ethyl]-3-(5-methyl-pyridin-2-yl)-N-(4-pyridin-2-yl- | | |
| | benzyl)-acrylamide | | |
| 16 | (S)-N-[1-Benzyl-2-(4-dimethylamino-piperidin-1-yl)-2- | 0.77 | 604.31 |
| | oxo-ethyl]-3-(6-methoxy-pyridin-3-yl)-N-(4-pyridin-2-yl- | | |
| | benzyl)-acrylamide | | |
| 17 | (S)-N-[1-Benzyl-2-(4-dimethylamino-piperidin-1-yl)-2- | 0.81 | 642.09 |
| | oxo-ethyl]-N-(4-pyridin-2-yl-benzyl)-3-(5-trifluoromethyl- | | |
| | pyridin-2-yl)-acrylamide | | |
| 18 | (S)-N-[1-Benzyl-2-(4-dimethylamino-piperidin-1-yl)-2- | 0.73 | 591.37 |
| | oxo-ethyl]-3-(1,5-dimethyl-1H-pyrazol-4-yl)-N-(4-pyridin- | | |
| | 2-yl-benzyl)-acrylamide | | |
| 19 | (S)-N-[1-Benzyl-2-(4-dimethylamino-piperidin-1-yl)-2- | 0.70 | 577.05 |
| | oxo-ethyl]-3-(1-methyl-1H-pyrazol-3-yl)-N-(4-pyridin-4- | | |
| | yl-benzyl)-acrylamide | | |
| 20 | (S)-N-[1-Benzyl-2-(4-dimethylamino-piperidin-1-yl)-2- | 0.73 | 591.47 |
| | oxo-ethyl]-3-(2,5-dimethyl-2H-pyrazol-3-yl)-N-(4-pyridin- | | |
| | 4-yl-benzyl)-acrylamide | | |
| 21 | (S)-N-[1-Benzyl-2-(4-dimethylamino-piperidin-1-yl)-2- | 0.72 | 605.18 |
| | oxo-ethyl]-N-(4-pyridin-4-yl-benzyl)-3-(1,3,5-trimethyl- | | |
| | 1H-pyrazol-4-yl)-acrylamide | | |
| 22 | (S)-N-[1-Benzyl-2-(4-dimethylamino-piperidin-1-yl)-2- | 0.73 | 607.40 |
| | oxo-ethyl]-3-(2,4-dimethyl-thiazol-5-yl)-N-(4-pyridin-4-yl- | | |
| | benzyl)-acrylamide | | |
| 23 | (S)-N-[1-Benzyl-2-(4-dimethylamino-piperidin-1-yl)-2- | 0.72 | 594.31 |
| | oxo-ethyl]-3-(2-methyl-thiazol-4-yl)-N-(4-pyridin-4-yl- | | |
| | benzyl)-acrylamide | | |
| 24 | (S)-N-[1-Benzyl-2-(4-dimethylamino-piperidin-1-yl)-2- | 0.73 | 592.91 |
| | oxo-ethyl]-3-(3,5-dimethyl-isoxazol-4-yl)-N-(4-pyridin-4- | | |
| | yl-benzyl)-acrylamide | | |
| 25 | (S)-N-[1-Benzyl-2-(4-dimethylamino-piperidin-1-yl)-2- | 0.72 | 592.63 |
| | oxo-ethyl]-3-(2,5-dimethyl-oxazol-4-yl)-N-(4-pyridin-4-yl- | | |
| | benzyl)-acrylamide | | |
| 26 | (S)-N-[1-Benzyl-2-(4-dimethylamino-piperidin-1-yl)-2- | 0.72 | 605.14 |
| | oxo-ethyl]-3-(6-methoxy-pyridazin-3-yl)-N-(4-pyridin-4- | | |
| | yl-benzyl)-acrylamide | | |
| 27 | (S)-N-[1-Benzyl-2-(4-dimethylamino-piperidin-1-yl)-2- | 0.65 | 588.34 |
| | oxo-ethyl]-3-(6-methyl-pyridin-3-yl)-N-(4-pyridin-4-yl- | | |
| | benzyl)-acrylamide | | |
| 28 | (S)-N-[1-Benzyl-2-(4-dimethylamino-piperidin-1-yl)-2- | 0.69 | 588.37 |
| | oxo-ethyl]-3-(5-methyl-pyridin-2-yl)-N-(4-pyridin-4-yl- | | |
| | benzyl)-acrylamide | | |
| 29 | (S)-N-[1-Benzyl-2-(4-dimethylamino-piperidin-1-yl)-2- | 0.74 | 604.21 |
| | oxo-ethyl]-3-(6-methoxy-pyridin-3-yl)-N-(4-pyridin-4-yl- | | |
| | benzyl)-acrylamide | | |
| 30 | (S)-N-[1-Benzyl-2-(4-dimethylamino-piperidin-1-yl)-2- | 0.78 | 642.09 |
| | oxo-ethyl]-N-(4-pyridin-4-yl-benzyl)-3-(5-trifluoromethyl- | | |
| | pyridin-2-yl)-acrylamide | | |
| 31 | (S)-N-[1-Benzyl-2-(4-dimethylamino-piperidin-1-yl)-2- | 0.71 | 591.27 |
| | oxo-ethyl]-3-(1,5-dimethyl-1H-pyrazol-4-yl)-N-(4-pyridin- | | |
| | 4-yl-benzyl)-acrylamide | | |
| 32 | (S)-N-[1-Benzyl-2-(4-dimethylamino-piperidin-1-yl)-2- | 0.71 | 605.45 |
| | oxo-ethyl]-3-(2-methoxy-pyrimidin-5-yl)-N-(4-pyridin-4- | | |
| | yl-benzyl)-acrylamide | | |
| 33 | (S)-N-[4-(4-Acetyl-piperazin-1-yl)-benzyl]-N-[1-benzyl-2- | 0.75 | 654.22 |
| | (4-dimethylamino-piperidin-1-yl)-2-oxo-ethyl]-3-(1,3,5- | | |
| | trimethyl-1H-pyrazol-4-yl)-acrylamide | | |
| 34 | (S)-N-[4-(4-Acetyl-piperazin-1-yl)-benzyl]-N-[1-benzyl-2- | 0.78 | 653.28 |
| | (4-dimethylamino-piperidin-1-yl)-2-oxo-ethyl]-3-(6- | | |
| | methoxy-pyridin-3-yl)-acrylamide | | |
| 35 | (S)-N-[4-(4-Acetyl-piperazin-1-yl)-benzyl]-N-[1-benzyl-2- | 0.82 | 691.23 |
| | (4-dimethylamino-piperidin-1-yl)-2-oxo-ethyl]-3-(5- | | |
| | trifluoromethyl-pyridin-2-yl)-acrylamide | | |
| 36 | (S)-N-[1-Benzyl-2-(4-dimethylamino-piperidin-1-yl)-2- | 0.80 | 606.18 |
| | oxo-ethyl]-N-(4-pyrimidin-5-yl-benzyl)-3-(1,3,5-trimethyl- | | |
| | 1H-pyrazol-4-yl)-acrylamide | | |
| 37 | (S)-N-[1-Benzyl-2-(4-dimethylamino-piperidin-1-yl)-2- | 0.81 | 609.20 |
| | oxo-ethyl]-3-(2,4-dimethyl-thiazol-5-yl)-N-(4-pyrimidin-5- | | |
| | yl-benzyl)-acrylamide | | |
| 38 | (S)-N-[1-Benzyl-2-(4-dimethylamino-piperidin-1-yl)-2- | 0.82 | 593.25 |
| | oxo-ethyl]-3-(3,5-dimethyl-isoxazol-4-yl)-N-(4-pyrimidin- | | |
| | 5-yl-benzyl)-acrylamide | | |
| 39 | (S)-N-[1-Benzyl-2-(4-dimethylamino-piperidin-1-yl)-2- | 0.85 | 609.18 |
| | oxo-ethyl]-3-(5-chloro-pyridin-2-yl)-N-(4-pyrimidin-5-yl- | | |
| | benzyl)-acrylamide | | |
| 40 | (S)-N-[1-Benzyl-2-(4-dimethylamino-piperidin-1-yl)-2- | 0.84 | 609.17 |
| | oxo-ethyl]-3-(6-chloro-pyridin-3-yl)-N-(4-pyrimidin-5-yl- | | |
| | benzyl)-acrylamide | | |
| 41 | (S)-N-[1-Benzyl-2-(4-dimethylamino-piperidin-1-yl)-2- | 0.83 | 605.22 |
| | oxo-ethyl]-3-(6-methoxy-pyridin-3-yl)-N-(4-pyrimidin-5- | | |
| | yl-benzyl)-acrylamide | | |
| 42 | (S)-N-[1-Benzyl-2-(4-dimethylamino-piperidin-1-yl)-2- | 0.88 | 642.88 |
| | oxo-ethyl]-N-(4-pyrimidin-5-yl-benzyl)-3-(5- | | |
| | trifluoromethyl-pyridin-2-yl)-acrylamide | | |
| 43 | (S)-N-[1-Benzyl-2-(4-dimethylamino-piperidin-1-yl)-2- | 0.79 | 592.28 |
| | oxo-ethyl]-3-(1,5-dimethyl-1H-pyrazol-4-yl)-N-(4- | | |
| | pyrimidin-5-yl-benzyl)-acrylamide | | |
| 44 | (S)-N-[1-Benzyl-2-(4-benzyl-piperidin-1-yl)-2-oxo-ethyl]- | 1.04 | 612.30 |
| | N-pyridin-2-ylmethyl-3-(4-trifluoromethyl-phenyl)- | | |
| | acrylamide | | |
| 45 | (S)-N-Benzyl-N-[1-benzyl-2-(4-benzyl-piperidin-1-yl)-2- | 1.23 | 611.10 |
| | oxo-ethyl]-3-(4-trifluoromethyl-phenyl)-acrylamide | | |
| 46 | (S)-N-[1-Benzyl-2-(4-benzyl-piperidin-1-yl)-2-oxo-ethyl]- | 1.10 | 688.32 |
| | N-(4-pyridin-2-yl-benzyl)-3-(4-trifluoromethyl-phenyl)- | | |
| | acrylamide | | |
| 47 | (S)-N-[1-Benzyl-2-(4-benzyl-piperidin-1-yl)-2-oxo-ethyl]- | 1.01 | 689.30 |
| | N-(4-pyridin-3-yl-benzyl)-3-(6-trifluoromethyl-pyridin-3- | | |
| | yl)-acrylamide | | |
| 48 | (S)-N-Benzyl-N-[1-benzyl-2-(4-benzyl-piperidin-1-yl)-2- | 1.16 | 612.30 |
| | oxo-ethyl]-3-(6-trifluoromethyl-pyridin-3-yl)-acrylamide | | |
| 49 | (S)-N-[1-Benzyl-2-oxo-2-(4-phenyl-piperidin-1-yl)-ethyl]- | 1.00 | 675.20 |
| | N-(4-pyrimidin-5-yl-benzyl)-3-(6-trifluoromethyl-pyridin- | | |
| | 3-yl)-acrylamide | | |
| 50 | (S)-N-[1-Benzyl-2-oxo-2-(4-phenyl-piperidin-1-yl)-ethyl]- | 0.99 | 675.22 |
| | N-(4-pyridin-2-yl-benzyl)-3-(6-trifluoromethyl-pyridin-3- | | |
| | yl)-acrylamide | | |
| 51 | (S)-N-[1-Benzyl-2-(4-dimethylaminomethyl-piperidin-1- | 0.77 | 656.32 |
| | yl)-2-oxo-ethyl]-N-(4-pyridin-2-yl-benzyl)-3-(6- | | |
| | trifluoromethyl-pyridin-3-yl)-acrylamide | | |
| 52 | (S)-N-[1-Benzyl-2-oxo-2-(4-pyrrolidin-1-ylmethyl- | 0.83 | 681.28 |
| | piperidin-1-yl)-ethyl]-N-(4-pyridin-2-yl-benzyl)-3-(4- | | |
| | trifluoromethyl-phenyl)-acrylamide | | |
| 53 | N-[(S)-1-Benzyl-2-((R)-3-dimethylamino-pyrrolidin-1-yl)- | 0.81 | 627.35 |
| | 2-oxo-ethyl]-N-(4-pyridin-2-yl-benzyl)-3-(4- | | |
| | trifluoromethyl-phenyl)-acrylamide | | |
| 54 | N-[(S)-1-Benzyl-2-((S)-3-dimethylamino-pyrrolidin-1-yl)- | 0.81 | 627.26 |
| | 2-oxo-ethyl]-N-(4-pyridin-2-yl-benzyl)-3-(4- | | |
| | trifluoromethyl-phenyl)-acrylamide | | |
| 55 | N-[(S)-1-Benzyl-2-((R)-3-dimethylamino-pyrrolidin-1-yl)- | 0.76 | 628.29 |
| | 2-oxo-ethyl]-N-(4-pyridin-2-yl-benzyl)-3-(6- | | |
| | trifluoromethyl-pyridin-3-yl)-acrylamide | | |
| 56 | N-[(S)-1-Benzyl-2-((S)-3-dimethylamino-pyrrolidin-1-yl)- | 0.76 | 628.25 |
| | 2-oxo-ethyl]-N-(4-pyridin-2-yl-benzyl)-3-(6- | | |
| | trifluoromethyl-pyridin-3-yl)-acrylamide | | |
| 57 | (S)-N-[1-Benzyl-2-(2,3-dihydro-indol-1-yl)-2-oxo-ethyl]- | 1.25 | 633.24 |
| | N-(4-pyrimidin-5-yl-benzyl)-3-(4-trifluoromethyl-phenyl)- | | |
| | acrylamide | | |
| 58 | (S)-N-[1-Benzyl-2-(1,3-dihydro-isoindol-2-yl)-2-oxo- | 0.97 | 633.23 |
| | ethyl]-N-(4-pyridin-2-yl-benzyl)-3-(6-trifluoromethyl- | | |
| | pyridin-3-yl)-acrylamide | | |
| 59 | (S)-3-(2,5-Dimethyl-2H-pyrazol-3-yl)-N-{1-[(2-methoxy- | 0.96 | 560.48 |
| | ethyl)-methyl-carbamoyl]-2-phenyl-ethyl}-N-(4- | | |
| | morpholin-4-yl-benzyl)-acrylamide | | |
| 60 | (S)-N-{1-[(2-Methoxy-ethyl)-methyl-carbamoyl]-2- | 0.94 | 574.45 |
| | phenyl-ethyl}-N-(4-morpholin-4-yl-benzyl)-3-(1,3,5- | | |
| | trimethyl-1H-pyrazol-4-yl)-acrylamide | | |
| 61 | (S)-3-(2,3-Dimethyl-3H-imidazol-4-yl)-N-{1-[(2-methoxy- | 0.77 | 560.43 |
| | ethyl)-methyl-carbamoyl]-2-phenyl-ethyl}-N-(4- | | |
| | morpholin-4-yl-benzyl)-acrylamide | | |
| 62 | (S)-N-{1-[(2-Methoxy-ethyl)-methyl-carbamoyl]-2- | 0.94 | 563.44 |
| | phenyl-ethyl}-3-(2-methyl-thiazol-4-yl)-N-(4-morpholin- | | |
| | 4-yl-benzyl)-acrylamide | | |
| 63 | (S)-3-(2,4-Dimethyl-thiazol-5-yl)-N-{1-[(2-methoxy- | 0.98 | 577.56 |
| | ethyl)-methyl-carbamoyl]-2-phenyl-ethyl}-N-(4- | | |
| | morpholin-4-yl-benzyl)-acrylamide | | |
| 64 | (S)-3-(3,5-Dimethyl-isoxazol-4-yl)-N-{1-[(2-methoxy- | 0.96 | 561.14 |
| | ethyl)-methyl-carbamoyl]-2-phenyl-ethyl}-N-(4- | | |
| | morpholin-4-yl-benzyl)-acrylamide | | |
| 65 | (S)-3-(2,5-Dimethyl-oxazol-4-yl)-N-{1-[(2-methoxy- | 0.93 | 561.23 |
| | ethyl)-methyl-carbamoyl]-2-phenyl-ethyl}-N-(4- | | |
| | morpholin-4-yl-benzyl)-acrylamide | | |
| 66 | (S)-N-{1-[(2-Methoxy-ethyl)-methyl-carbamoyl]-2- | 0.94 | 550.16 |
| | phenyl-ethyl}-N-(4-morpholin-4-yl-benzyl)-3- | | |
| | [1,2,3]thiadiazol-4-yl-acrylamide | | |
| 67 | (S)-N-{1-[(2-Methoxy-ethyl)-methyl-carbamoyl]-2- | 1.03 | 611.18 |
| | phenyl-ethyl}-N-(4-morpholin-4-yl-benzyl)-3-(5- | | |
| | trifluoromethyl-pyridin-2-yl)-acrylamide | | |
| 68 | (S)-N-{1-[(2-Methoxy-ethyl)-methyl-carbamoyl]-2- | 0.84 | 557.37 |
| | phenyl-ethyl}-3-(5-methyl-pyridin-2-yl)-N-(4-morpholin- | | |
| | 4-yl-benzyl)-acrylamide | | |
| 69 | (S)-N-{1-[(2-Methoxy-ethyl)-methyl-carbamoyl]-2- | 0.78 | 557.29 |
| | phenyl-ethyl}-3-(6-methyl-pyridin-3-yl)-N-(4-morpholin- | | |
| | 4-yl-benzyl)-acrylamide | | |
| 70 | (S)-3-(6-Chloro-pyridin-3-yl)-N-{1-[(2-methoxy-ethyl)- | 0.98 | 576.77 |
| | methyl-carbamoyl]-2-phenyl-ethyl}-N-(4-morpholin-4-yl- | | |
| | benzyl)-acrylamide | | |
| 71 | (S)-N-{1-[(2-Methoxy-ethyl)-methyl-carbamoyl]-2- | 0.98 | 573.32 |
| | phenyl-ethyl}-3-(6-methoxy-pyridin-3-yl)-N-(4- | | |
| | morpholin-4-yl-benzyl)-acrylamide | | |
| 72 | (S)-N-{1-[(2-Methoxy-ethyl)-methyl-carbamoyl]-2- | 0.92 | 574.14 |
| | phenyl-ethyl}-3-(2-methoxy-pyrimidin-5-yl)-N-(4- | | |
| | morpholin-4-yl-benzyl)-acrylamide | | |
| 73 | (S)-N-{1-[(2-Methoxy-ethyl)-methyl-carbamoyl]-2- | 0.78 | 538.19 |
| | phenyl-ethyl}-3-(1-methyl-1H-pyrazol-3-yl)-N-(4-pyridin- | | |
| | 2-yl-benzyl)-acrylamide | | |
| 74 | (S)-3-(2,5-Dimethyl-2H-pyrazol-3-yl)-N-{1-[(2-methoxy- | 0.82 | 552.22 |
| | ethyl)-methyl-carbamoyl]-2-phenyl-ethyl}-N-(4-pyridin-2- | | |
| | yl-benzyl)-acrylamide | | |
| 75 | (S)-N-{1-[(2-Methoxy-ethyl)-methyl-carbamoyl]-2- | 0.79 | 566.21 |
| | phenyl-ethyl}-N-(4-pyridin-2-yl-benzyl)-3-(1,3,5- | | |
| | trimethyl-1H-pyrazol-4-yl)-acrylamide | | |
| 76 | (S)-3-(2,3-Dimethyl-3H-imidazol-4-yl)-N-{1-[(2-methoxy- | 0.67 | 552.22 |
| | ethyl)-methyl-carbamoyl]-2-phenyl-ethyl}-N-(4-pyridin-2- | | |
| | yl-benzyl)-acrylamide | | |
| 77 | (S)-3-(2,4-Dimethyl-thiazol-5-yl)-N-{1-[(2-methoxy- | 0.85 | 569.15 |
| | ethyl)-methyl-carbamoyl]-2-phenyl-ethyl}-N-(4-pyridin-2- | | |
| | yl-benzyl)-acrylamide | | |
| 78 | (S)-N-{1-[(2-Methoxy-ethyl)-methyl-carbamoyl]-2- | 0.82 | 555.15 |
| | phenyl-ethyl}-3-(2-methyl-thiazol-4-yl)-N-(4-pyridin-2-yl- | | |
| | benzyl)-acrylamide | | |
| 79 | (S)-N-{1-[(2-Methoxy-ethyl)-methyl-carbamoyl]-2- | 0.83 | 539.19 |
| | phenyl-ethyl}-3-(5-methyl-isoxazol-3-yl)-N-(4-pyridin-2- | | |
| | yl-benzyl)-acrylamide | | |
| 80 | (S)-3-(3,5-Dimethyl-isoxazol-4-yl)-N-{1-[(2-methoxy- | 0.83 | 553.18 |
| | ethyl)-methyl-carbamoyl]-2-phenyl-ethyl}-N-(4-pyridin-2- | | |
| | yl-benzyl)-acrylamide | | |
| 81 | (S)-3-(2,5-Dimethyl-oxazol-4-yl)-N-{1-[(2-methoxy- | 0.81 | 553.19 |
| | ethyl)-methyl-carbamoyl]-2-phenyl-ethyl}-N-(4-pyridin-2- | | |
| | yl-benzyl)-acrylamide | | |
| 82 | (S)-N-{1-[(2-Methoxy-ethyl)-methyl-carbamoyl]-2- | 0.75 | 566.19 |
| | phenyl-ethyl}-3-(6-methoxy-pyridazin-3-yl)-N-(4-pyridin- | | |
| | 2-yl-benzyl)-acrylamide | | |
| 83 | (S)-N-{1-[(2-Methoxy-ethyl)-methyl-carbamoyl]-2- | 0.78 | 550.20 |
| | phenyl-ethyl}-3-(2-methyl-pyrimidin-5-yl)-N-(4-pyridin-2- | | |
| | yl-benzyl)-acrylamide | | |
| 84 | (S)-N-{1-[(2-Methoxy-ethyl)-methyl-carbamoyl]-2- | 0.69 | 549.19 |
| | phenyl-ethyl}-3-(6-methyl-pyridin-3-yl)-N-(4-pyridin-2-yl- | | |
| | benzyl)-acrylamide | | |
| 85 | (S)-3-(6-Chloro-pyridin-3-yl)-N-{1-[(2-methoxy-ethyl)- | 0.85 | 569.16 |
| | methyl-carbamoyl]-2-phenyl-ethyl}-N-(4-pyridin-2-yl- | | |
| | benzyl)-acrylamide | | |
| 86 | (S)-N-{1-[(2-Methoxy-ethyl)-methyl-carbamoyl]-2- | 0.74 | 549.20 |
| | phenyl-ethyl}-3-(5-methyl-pyridin-2-yl)-N-(4-pyridin-2-yl- | | |
| | benzyl)-acrylamide | | |
| 87 | (S)-N-{1-[(2-Methoxy-ethyl)-methyl-carbamoyl]-2- | 0.84 | 565.20 |
| | phenyl-ethyl}-3-(6-methoxy-pyridin-3-yl)-N-(4-pyridin-2- | | |
| | yl-benzyl)-acrylamide | | |
| 88 | (S)-N-{1-[(2-Methoxy-ethyl)-methyl-carbamoyl]-2- | 0.88 | 604.14 |
| | phenyl-ethyl}-N-(4-pyridin-2-yl-benzyl)-3-(2- | | |
| | trifluoromethyl-pyrimidin-5-yl)-acrylamide | | |
| 89 | (S)-N-{1-[(2-Methoxy-ethyl)-methyl-carbamoyl]-2- | 0.89 | 603.14 |
| | phenyl-ethyl}-N-(4-pyridin-2-yl-benzyl)-3-(5- | | |
| | trifluoromethyl-pyridin-2-yl)-acrylamide | | |
| 90 | (S)-3-(1,5-Dimethyl-1H-pyrazol-4-yl)-N-{1-[(2-methoxy- | 0.80 | 552.19 |
| | ethyl)-methyl-carbamoyl]-2-phenyl-ethyl}-N-(4-pyridin-2- | | |
| | yl-benzyl)-acrylamide | | |
| 91 | (S)-N-{1-[(2-Methoxy-ethyl)-methyl-carbamoyl]-2- | 0.76 | 538.20 |
| | phenyl-ethyl}-3-(1-methyl-1H-pyrazol-3-yl)-N-(4-pyridin- | | |
| | 4-yl-benzyl)-acrylamide | | |
| 92 | (S)-3-(2,3-Dimethyl-3H-imidazol-4-yl)-N-{1-[(2-methoxy- | 0.65 | 552.15 |
| | ethyl)-methyl-carbamoyl]-2-phenyl-ethyl}-N-(4-pyridin-4- | | |
| | yl-benzyl)-acrylamide | | |
| 93 | (S)-3-(2,4-Dimethyl-thiazol-5-yl)-N-{1-[(2-methoxy- | 0.77 | 569.06 |
| | ethyl)-methyl-carbamoyl]-2-phenyl-ethyl}-N-(4-pyridin-4- | | |
| | yl-benzyl)-acrylamide | | |
| 94 | (S)-N-{1-[(2-Methoxy-ethyl)-methyl-carbamoyl]-2- | 0.80 | 555.15 |
| | phenyl-ethyl}-3-(2-methyl-thiazol-4-yl)-N-(4-pyridin-4-yl- | | |
| | benzyl)-acrylamide | | |
| 95 | (S)-N-{1-[(2-Methoxy-ethyl)-methyl-carbamoyl]-2- | 0.81 | 539.17 |
| | phenyl-ethyl}-3-(5-methyl-isoxazol-3-yl)-N-(4-pyridin-4- | | |
| | yl-benzyl)-acrylamide | | |
| 96 | (S)-3-(3,5-Dimethyl-isoxazol-4-yl)-N-{1-[(2-methoxy- | 0.81 | 553.18 |
| | ethyl)-methyl-carbamoyl]-2-phenyl-ethyl}-N-(4-pyridin-4- | | |
| | yl-benzyl)-acrylamide | | |
| 97 | (S)-3-(2,5-Dimethyl-oxazol-4-yl)-N-{1-[(2-methoxy- | 0.79 | 553.18 |
| | ethyl)-methyl-carbamoyl]-2-phenyl-ethyl}-N-(4-pyridin-4- | | |
| | yl-benzyl)-acrylamide | | |
| 98 | (S)-N-{1-[(2-Methoxy-ethyl)-methyl-carbamoyl]-2- | 0.78 | 542.12 |
| | phenyl-ethyl}-N-(4-pyridin-4-yl-benzyl)-3- | | |
| | [1,2,3]thiadiazol-4-yl-acrylamide | | |
| 99 | (S)-3-(6-Chloro-pyridin-3-yl)-N-{1-[(2-methoxy-ethyl)- | 0.83 | 569.14 |
| | methyl-carbamoyl]-2-phenyl-ethyl}-N-(4-pyridin-4-yl- | | |
| | benzyl)-acrylamide | | |
| 100 | (S)-N-{1-[(2-Methoxy-ethyl)-methyl-carbamoyl]-2- | 0.72 | 549.23 |
| | phenyl-ethyl}-3-(5-methyl-pyridin-2-yl)-N-(4-pyridin-4-yl- | | |
| | benzyl)-acrylamide | | |
| 101 | (S)-N-{1-[(2-Methoxy-ethyl)-methyl-carbamoyl]-2- | 0.82 | 565.22 |
| | phenyl-ethyl}-3-(6-methoxy-pyridin-3-yl)-N-(4-pyridin-4- | | |
| | yl-benzyl)-acrylamide | | |
| 102 | (S)-N-{1-[(2-Methoxy-ethyl)-methyl-carbamoyl]-2- | 0.86 | 603.16 |
| | phenyl-ethyl}-N-(4-pyridin-4-yl-benzyl)-3-(5- | | |
| | trifluoromethyl-pyridin-2-yl)-acrylamide | | |
| 103 | (S)-N-[4-(4-Acetyl-piperazin-1-yl)-benzyl]-N-{1-[(2- | 0.86 | 587.20 |
| | methoxy-ethyl)-methyl-carbamoyl]-2-phenyl-ethyl}-3-(1- | | |
| | methyl-1H-pyrazol-3-yl)-acrylamide | | |
| 104 | (S)-N-[4-(4-Acetyl-piperazin-1-yl)-benzyl]-3-(2,5- | 0.91 | 601.23 |
| | dimethyl-2H-pyrazol-3-yl)-N-{1-[(2-methoxy-ethyl)- | | |
| | methyl-carbamoyl]-2-phenyl-ethyl}-acrylamide | | |
| 105 | (S)-N-[4-(4-Acetyl-piperazin-1-yl)-benzyl]-N-{1-[(2- | 0.90 | 615.25 |
| | methoxy-ethyl)-methyl-carbamoyl]-2-phenyl-ethyl}-3- | | |
| | (1,3,5-trimethyl-1H-pyrazol-4-yl)-acrylamide | | |
| 106 | (S)-N-[4-(4-Acetyl-piperazin-1-yl)-benzyl]-3-(2,3- | 0.73 | 601.24 |
| | dimethyl-3H-imidazol-4-yl)-N-{1-[(2-methoxy-ethyl)- | | |
| | methyl-carbamoyl]-2-phenyl-ethyl}-acrylamide | | |
| 107 | (S)-N-[4-(4-Acetyl-piperazin-1-yl)-benzyl]-3-(2,4- | 0.94 | 618.11 |
| | dimethyl-thiazol-5-yl)-N-{1-[(2-methoxy-ethyl)-methyl- | | |
| | carbamoyl]-2-phenyl-ethyl}-acrylamide | | |
| 108 | (S)-N-[4-(4-Acetyl-piperazin-1-yl)-benzyl]-N-{1-[(2- | 0.91 | 604.20 |
| | methoxy-ethyl)-methyl-carbamoyl]-2-phenyl-ethyl}-3-(2- | | |
| | methyl-thiazol-4-yl)-acrylamide | | |
| 109 | (S)-N-[4-(4-Acetyl-piperazin-1-yl)-benzyl]-3-(3,5- | 0.93 | 602.23 |
| | dimethyl-isoxazol-4-yl)-N-{1-[(2-methoxy-ethyl)-methyl- | | |
| | carbamoyl]-2-phenyl-ethyl}-acrylamide | | |
| 110 | (S)-N-[4-(4-Acetyl-piperazin-1-yl)-benzyl]-3-(2,5- | 0.90 | 602.23 |
| | dimethyl-oxazol-4-yl)-N-{1-[(2-methoxy-ethyl)-methyl- | | |
| | carbamoyl]-2-phenyl-ethyl}-acrylamide | | |
| 111 | (S)-N-[4-(4-Acetyl-piperazin-1-yl)-benzyl]-N-{1-[(2- | 0.88 | 615.20 |
| | methoxy-ethyl)-methyl-carbamoyl]-2-phenyl-ethyl}-3-(6- | | |
| | methoxy-pyridazin-3-yl)-acrylamide | | |
| 112 | (S)-N-[4-(4-Acetyl-piperazin-1-yl)-benzyl]-N-{1-[(2- | 0.75 | 598.29 |
| | methoxy-ethyl)-methyl-carbamoyl]-2-phenyl-ethyl}-3-(6- | | |
| | methyl-pyridin-3-yl)-acrylamide | | |
| 113 | (S)-N-[4-(4-Acetyl-piperazin-1-yl)-benzyl]-3-(6-chloro- | 0.95 | 618.14 |
| | pyridin-3-yl)-N-{1-[(2-methoxy-ethyl)-methyl-carbamoyl]- | | |
| | 2-phenyl-ethyl}-acrylamide | | |
| 114 | (S)-N-[4-(4-Acetyl-piperazin-1-yl)-benzyl]-N-{1-[(2- | 0.80 | 598.21 |
| | methoxy-ethyl)-methyl-carbamoyl]-2-phenyl-ethyl}-3-(5- | | |
| | methyl-pyridin-2-yl)-acrylamide | | |
| 115 | (S)-N-[4-(4-Acetyl-piperazin-1-yl)-benzyl]-N-{1-[(2- | 0.94 | 614.21 |
| | methoxy-ethyl)-methyl-carbamoyl]-2-phenyl-ethyl}-3-(6- | | |
| | methoxy-pyridin-3-yl)-acrylamide | | |
| 116 | (S)-N-[4-(4-Acetyl-piperazin-1-yl)-benzyl]-N-{1-[(2- | 0.99 | 652.18 |
| | methoxy-ethyl)-methyl-carbamoyl]-2-phenyl-ethyl}-3-(5- | | |
| | trifluoromethyl-pyridin-2-yl)-acrylamide | | |
| 117 | (S)-N-[4-(4-Acetyl-piperazin-1-yl)-benzyl]-3-(1,5- | 0.88 | 601.24 |
| | dimethyl-1H-pyrazol-4-yl)-N-{1-[(2-methoxy-ethyl)- | | |
| | methyl-carbamoyl]-2-phenyl-ethyl}-acrylamide | | |
| 118 | (S)-N-[4-(4-Acetyl-piperazin-1-yl)-benzyl]-N-{1-[(2- | 0.88 | 615.21 |
| | methoxy-ethyl)-methyl-carbamoyl]-2-phenyl-ethyl}-3-(2- | | |
| | methoxy-pyrimidin-5-yl)-acrylamide | | |
| 119 | (S)-N-{1-[(2-Methoxy-ethyl)-methyl-carbamoyl]-2- | 0.95 | 539.18 |
| | phenyl-ethyl}-3-(1-methyl-1H-pyrazol-3-yl)-N-(4- | | |
| | pyrimidin-5-yl-benzyl)-acrylamide | | |
| 120 | (S)-3-(2,3-Dimethyl-3H-imidazol-4-yl)-N-{1-[(2-methoxy- | 0.76 | 553.20 |
| | ethyl)-methyl-carbamoyl]-2-phenyl-ethyl}-N-(4- | | |
| | pyrimidin-5-yl-benzyl)-acrylamide | | |
| 121 | (S)-3-(2,4-Dimethyl-thiazol-5-yl)-N-{1-[(2-methoxy- | 1.05 | 570.17 |
| | ethyl)-methyl-carbamoyl]-2-phenyl-ethyl}-N-(4- | | |
| | pyrimidin-5-yl-benzyl)-acrylamide | | |
| 122 | (S)-N-{1-[(2-Methoxy-ethyl)-methyl-carbamoyl]-2- | 1.00 | 556.13 |
| | phenyl-ethyl}-3-(2-methyl-thiazol-4-yl)-N-(4-pyrimidin-5- | | |
| | yl-benzyl)-acrylamide | | |
| 123 | (S)-N-{1-[(2-Methoxy-ethyl)-methyl-carbamoyl]-2- | 1.01 | 540.17 |
| | phenyl-ethyl}-3-(5-methyl-isoxazol-3-yl)-N-(4-pyrimidin- | | |
| | 5-yl-benzyl)-acrylamide | | |
| 124 | (S)-3-(3,5-Dimethyl-isoxazol-4-yl)-N-{1-[(2-methoxy- | 1.03 | 554.17 |
| | ethyl)-methyl-carbamoyl]-2-phenyl-ethyl}-N-(4- | | |
| | pyrimidin-5-yl-benzyl)-acrylamide | | |
| 125 | (S)-N-{1-[(2-Methoxy-ethyl)-methyl-carbamoyl]-2- | 0.99 | 542.84 |
| | phenyl-ethyl}-N-(4-pyrimidin-5-yl-benzyl)-3- | | |
| | [1,2,3]thiadiazol-4-yl-acrylamide | | |
| 126 | (S)-N-{1-[(2-Methoxy-ethyl)-methyl-carbamoyl]-2- | 0.97 | 567.03 |
| | phenyl-ethyl}-3-(6-methoxy-pyridazin-3-yl)-N-(4- | | |
| | pyrimidin-5-yl-benzyl)-acrylamide | | |
| 127 | (S)-3-(5-Chloro-pyridin-2-yl)-N-{1-[(2-methoxy-ethyl)- | 1.07 | 570.15 |
| | methyl-carbamoyl]-2-phenyl-ethyl}-N-(4-pyrimidin-5-yl- | | |
| | benzyl)-acrylamide | | |
| 128 | (S)-3-(6-Chloro-pyridin-3-yl)-N-{1-[(2-methoxy-ethyl)- | 1.05 | 570.12 |
| | methyl-carbamoyl]-2-phenyl-ethyl}-N-(4-pyrimidin-5-yl- | | |
| | benzyl)-acrylamide | | |
| 129 | (S)-N-{1-[(2-Methoxy-ethyl)-methyl-carbamoyl]-2- | 1.04 | 566.10 |
| | phenyl-ethyl}-3-(2-methoxy-pyrimidin-5-yl)-N-(4- | | |
| | pyrimidin-5-yl-benzyl)-acrylamide | | |
| 130 | (S)-N-{1-[(2-Methoxy-ethyl)-methyl-carbamoyl]-2- | 1.09 | 604.13 |
| | phenyl-ethyl}-N-(4-pyrimidin-5-yl-benzyl)-3-(5- | | |
| | trifluoromethyl-pyridin-2-yl)-acrylamide | | |
| 131 | (S)-3-(2,3-Dimethyl-3H-imidazol-4-yl)-N-{1-[(2-methoxy- | 0.66 | 552.18 |
| | ethyl)-methyl-carbamoyl]-2-phenyl-ethyl}-N-(4-pyridin-3- | | |
| | yl-benzyl)-acrylamide | | |
| 132 | (S)-3-(2,4-Dimethyl-thiazol-5-yl)-N-{1-[(2-methoxy- | 0.83 | 569.12 |
| | ethyl)-methyl-carbamoyl]-2-phenyl-ethyl}-N-(4-pyridin-3- | | |
| | yl-benzyl)-acrylamide | | |
| 133 | (S)-3-(2,5-Dimethyl-oxazol-4-yl)-N-{1-[(2-methoxy- | 0.81 | 553.16 |
| | ethyl)-methyl-carbamoyl]-2-phenyl-ethyl}-N-(4-pyridin-3- | | |
| | yl-benzyl)-acrylamide | | |
| 134 | (S)-3-(6-Chloro-pyridin-3-yl)-N-{1-[(2-methoxy-ethyl)- | 0.84 | 569.10 |
| | methyl-carbamoyl]-2-phenyl-ethyl}-N-(4-pyridin-3-yl- | | |
| | benzyl)-acrylamide | | |
| 135 | (S)-N-{1-[(2-Methoxy-ethyl)-methyl-carbamoyl]-2- | 0.83 | 565.16 |
| | phenyl-ethyl}-3-(6-methoxy-pyridin-3-yl)-N-(4-pyridin-3- | | |
| | yl-benzyl)-acrylamide | | |

*Analytic A, Zorbax SB-AQ column, acidic conditions

### Preparation of Compounds of Formula I Via Method B:

### Step 1

### General Procedure 1

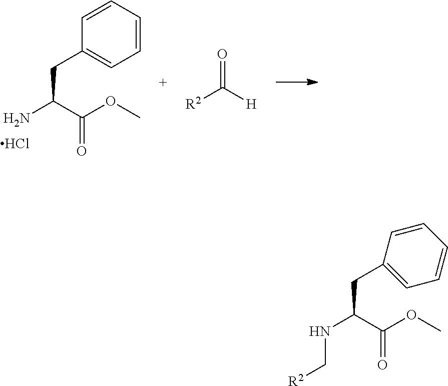

To a solution of L-phenylalanine-methylester hydrochloride (1 eq), and TEA (1 eq) in dry MeOH (5 mL/mmol) was added in one portion the aldehyde R²CHO (1 eq). The resulting mixture was refluxed overnight under nitrogen atmosphere. After cooling to 0° C., sodium borohydride (1.5 eq) was added portionwise. The reaction mixture was stirred at rt for 1 h, then quenched with a sat. NaHCO₃ solution and extracted twice with EA. The combined organic extracts were washed with brine, dried (MgSO₄), filtered and concentrated under reduced pressure to afford the crude secondary amine, which was used for the next step without further purification.

| | | | LC-MS* | |
|---|---|---|---|---|
| | | | t_{R} | |
| R² | Chemical name | Yield | (min) | [M + H]⁺ |
| 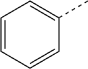 | (S)-2-Benzylamino-3-phenyl- propionic acid methyl ester | 14% | 0.78 | 270.15 |
| 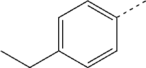 | (S)-2-(4-Ethyl-benzylamino)-3- phenyl-propionic acid methyl ester | 11% | 0.83 | 298.18 |
| 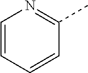 | (S)-3-Phenyl-2-[(pyridin-2-ylmethyl)- amino]-propionic acid methyl ester | 47% | 0.72 | 271.16 |
| 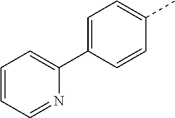 | (S)-3-Phenyl-2-(4-pyridin-2-yl- benzylamino)-propionic acid methyl ester | 99% | 0.70 | 347.40 |
| 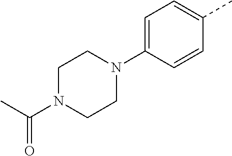 | (S)-2-[4-(4-Acetyl-piperazin-1-yl)- benzylamino]-3-phenyl-propionic acid methyl ester | 89% | 0.74 | 396.40 |
| 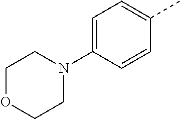 | (S)-2-(4-Morpholin-4-yl- benzylamino)-3-phenyl-propionic acid methyl ester | 84% | 0.76 | 355.30 |
| 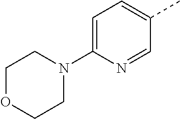 | (S)-2-[(6-Morpholin-4-yl-pyridin-3- ylmethyl)-amino]-3-phenyl-propionic acid methyl ester | 89% | 0.81 | 356.08 |

*Analytic A, Zorbax SB-AQ column, acidic conditions

### General Procedure 2

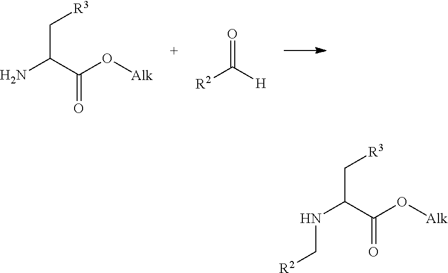

To a solution of the amino-acid methyl/ethyl ester (1 eq) in dry MeOH (5 mL/mmol) at 0° C. were added successively the aldehyde R²CHO (1 eq), sodium cyanoborohydride (1 eq) and acetic acid (1 eq). The reaction mixture was stirred at rt for 1 h under nitrogen atmosphere and then concentrated in vacuo. The resulting residue was dissolved in a small amount of water, basified with a 10% Na₂CO₃ solution and extracted twice with DCM. The combined organic extracts were dried (MgSO₄) and concentrated under reduced pressure to afford the crude secondary amine, which was used for the next step without further purification.

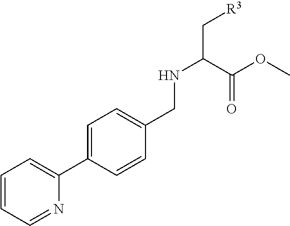

| | | LC-MS* | |
|---|---|---|---|
| | | t_{R} | |
| R³ | Chemical name | (min) | [M + H]⁺ |
| 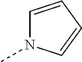 | (S)-2-(4-Pyridin-2-yl- benzylamino)-3-pyrrol-1-yl- propionic acid methyl ester | 2.20 | 336.30 |
| 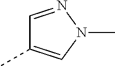 | rac-3-(1-Methyl-1H-pyrazol- 4-yl)-2-(4-pyridin-2-yl- benzylamino)-propionic acid methyl ester | 1.36 | 351.10 |
| 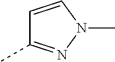 | rac-3-(1-Methyl-1H-pyrazol- 3-yl)-2-(4-pyridin-2-yl- benzylamino)-propionic acid methyl ester | 1.83 | 351.20 |
| 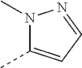 | rac-3-(2-Methyl-2H-pyrazol- 3-yl)-2-(4-pyridin-2-yl- benzylamino)-propionic acid methyl ester | 1.82 | 351.20 |

*Analytic B

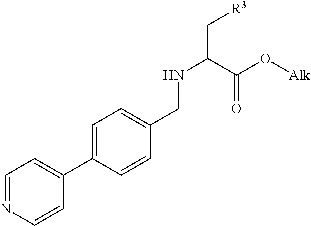

| | | | LC-MS* | |
|---|---|---|---|---|
| | | | t_{R} | |
| R³ | Alk | Chemical name | (min) | [M + H]⁺ |
| 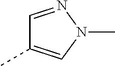 | Me | rac-3-(1-Methyl-1H- pyrazol-4-yl)-2-(4- pyridin- 4-yl-benzylamino)- propionic acid methyl ester | 0.61 | 351.10 |
| 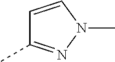 | Me | rac-3-(1-Methyl-1H- pyrazol-3-yl)-2-(4- pyridin-4-yl-benzylamino)- propionic acid methyl ester | 0.61 | 351.20 |
| 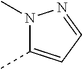 | Me | rac-3-(2-Methyl-2H- pyrazol-3-yl)-2-(4- pyridin- 4-yl-benzylamino)- propionic acid methyl ester | 0.61 | 351.10 |
| 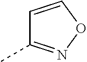 | Et | rac-3-Isoxazol-3-yl-2- (4-pyridin-4-yl- benzylamino)-propionic acid ethyl ester | 1.02 | 352.30 |
| 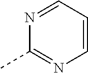 | Et | rac-2-(4-Pyridin-4- yl-benzylamino)-3- pyrimidin-2-yl-propionic acid ethyl ester | 0.60 | 363.20 |

*Analytic B

### Step 2

### General Procedure 1

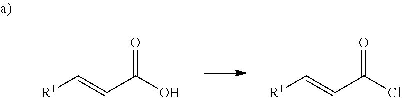

To an ice-cooled solution of the cinnamic acid (1 eq) in a mixture of dry DCM (1 mL/mmol) and DMF (few drops) was added dropwise oxalyl chloride (1.1 eq). The reaction mixture was stirred at 0° C. for 3 h and concentrated in vacuo to yield the crude acid chloride.

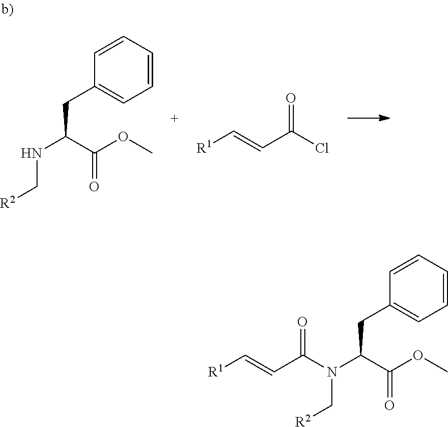

To an ice-cooled solution of the secondary amine (1 eq) and DIPEA (2 eq) in dry DCM (4 mL/mmol) was added dropwise a solution of the acid chloride (1 eq) in dry DCM (4 mL/mmol). The reaction mixture was stirred at 0° C. for 1 h and then concentrated in vacuo. The resulting residue was taken up in EA, washed with brine, dried (MgSO₄), filtered and concentrated under reduced pressure. FC (n-heptane/EA or DCM/MeOH system) afforded the pure amide.

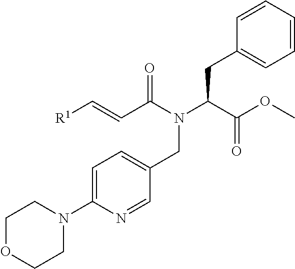

| | | | LC-MS | | |
|---|---|---|---|---|---|
| | | | t_{R} | | |
| R¹ | Chemical name | Yield | (min) | [M + H]⁺ | conditions |
| 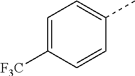 | (S)-2-{(6-Morpholin-4-yl-pyridin- 3-ylmethyl)-[3-(4-trifluoromethyl- phenyl)-acryloyl]-amino}-3- phenyl-propionic acid methyl ester | 45% | 0.93 | 554.21 | analytic A Zorbax SB-AQ acidic |
| 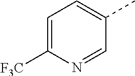 | (S)-2-{(6-Morpholin-4-yl-pyridin- 3-ylmethyl)-[3-(6-trifluoromethyl- pyridin-3-yl)-acryloyl]-amino}-3- phenyl-propionic acid methyl ester | 79% | 0.91 | 554.79 | analytic A Zorbax Extend basic |
| 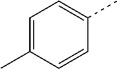 | (S)-2-[(6-Morpholin-4-yl-pyridin- 3-ylmethyl)-(3-p-tolyl-acryloyl)- amino]-3-phenyl-propionic acid methyl ester | crude | 0.84 | 500.26 | analytic A Zorbax SB-AQ acidic |
| 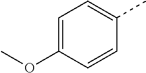 | (S)-2-[[3-(4-Methoxy-phenyl)- acryloyl]-(6-morpholin-4-yl- pyridin-3-ylmethyl)-amino]-3- phenyl-propionic acid methyl ester | quant. | 0.91 | 516.02 | analytic A Zorbax Extend basic |

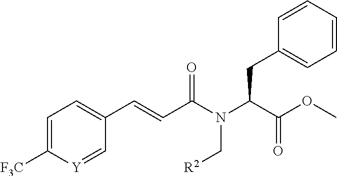

| | | | | LC-MS* | |
|---|---|---|---|---|---|
| | | | | t_{R} | |
| R² | Y | Chemical name | Yield | (min) | [M + H]⁺ |
| 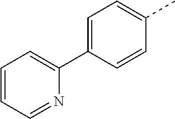 | CH | (S)-3-Phenyl-2-{(4-pyridin-2-yl- benzyl)-[3-(4-trifluoromethyl- phenyl)-acryloyl]-amino}-propionic acid methyl ester | 89% | 0.96 | 545.32 |
| 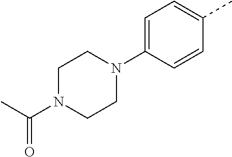 | CH | (S)-2-{[4-(4-Acetyl-piperazin-1-yl)- benzyl]-[3-(4-trifluoromethyl- phenyl)-acryloyl]-amino}-3-phenyl- propionic acid methyl ester | 70% | 1.12 | 594.35 |
| 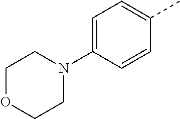 | CH | (S)-2-{(4-Morpholin-4-yl-benzyl)- [3-(4-trifluoromethyl-phenyl)- acryloyl]-amino}-3-phenyl- propionic acid methyl ester | 74% | 1.12 | 553.14 |
| 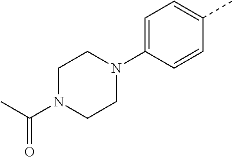 | N | (S)-2-{[4-(4-Acetyl-piperazin-1-yl)- benzyl]-[3-(6-trifluoromethyl- pyridin-3-yl)-acryloyl]-amino}-3- phenyl-propionic acid methyl ester | 34% | 1.06 | 595.24 |
| 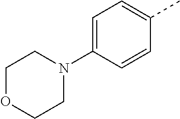 | N | (S)-2-{(4-Morpholin-4-yl-benzyl)- [3-(6-trifluoromethyl-pyridin-3-yl)- acryloyl]-amino}-3-phenyl- propionic acid methyl ester | 39% | 1.07 | 553.91 |

*Analytic A, Zorbax SB-AQ column, acidic conditions

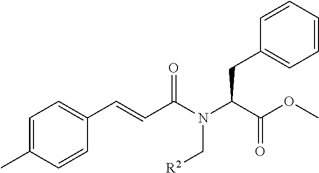

| | | | LC-MS* | |
|---|---|---|---|---|
| | | | t_{R} | |
| R² | Chemical name | Yield | (min) | [M + H]⁺ |
| 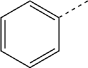 | (S)-2-[Benzyl-(3- p-tolyl-acryloyl)- amino]-3- phenyl-propionic acid methyl ester | 81% | 1.14 | 414.10 |
| 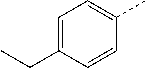 | (S)-2-[(4-Ethyl- benzyl)- (3-p-tolyl-acryloyl)- amino]-3-phenyl- propionic acid methyl ester | 95% | 1.18 | 442.10 |
| 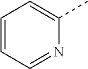 | (S)-3-Phenyl-2- [pyridin- 2-ylmethyl-(3-p- tolyl-acryloyl)- amino]-propionic acid methyl ester | 96% | 0.91 | 415.05 |

*Analytic A, Zorbax SB-AQ column, acidic conditions

### General Procedure 2

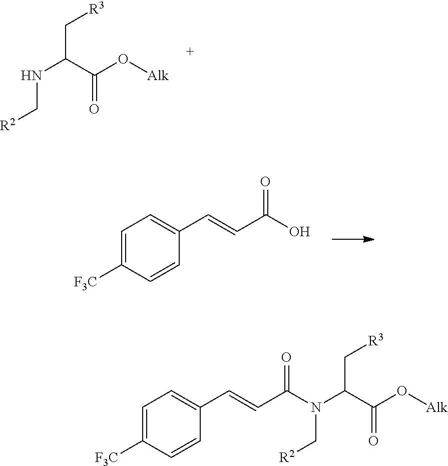

To an ice-cooled solution of 4-(trifluoromethyl)cinnamic acid (1.05 eq) in a mixture of dry DCM (2 mL/mmol) and DMF (few drops) was added dropwise oxalyl chloride (1.1 eq). The reaction mixture was stirred at 0° C. for 15 min under nitrogen atmosphere, then allowed to warm at rt for 30 min.

To this mixture at 0° C. was added a solution of the secondary amine (1 eq), TEA (2 eq) and DMAP (0.05 eq) in dry DCM (2 mL/mmol). The reaction mixture was stirred at rt overnight under nitrogen atmosphere, then diluted with DCM and washed with water. The aq. phase was separated and extracted with DCM. The combined organic extracts were washed with a sat. NaHCO₃ solution, dried (MgSO₄), filtered and concentrated under reduced pressure. FC (n-heptane/EA or EA/MeOH system) afforded the pure amide.

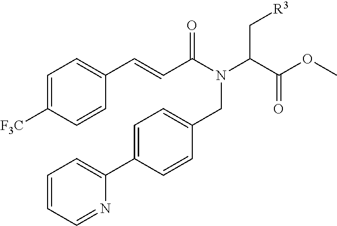

| | | | LC-MS* | |
|---|---|---|---|---|
| | | | t_{R} | |
| R³ | Chemical name | Yield | (min) | [M + H]⁺ |
| 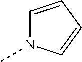 | (S)-2-{(4-Pyridin- 2-yl-benzyl)-[3-(4- trifluoromethyl-phenyl)- acryloyl]-amino}-3- pyrrol-1-yl-propionic acid methyl ester | 72% | 3.58 | 534.30 |
| 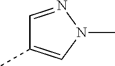 | rac-3-(1-Methyl-1H- pyrazol-4-yl)-2-{(4- pyridin-2-yl-benzyl)-[3- (trifluoromethyl-phenyl)- acryloyl]-amino}-propionic acid methyl ester | 76% | 3.17 | 549.30 |
| 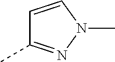 | rac-3-(1-Methyl-1H- pyrazol-3-yl)-2-{(4- pyridin-2-yl-benzyl)-[3- (4-trifluoromethyl-phenyl)- acryloyl]-amino}-propionic acid methyl ester | 69% | 3.20 | 549.30 |
| 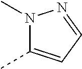 | rac-3-(2-Methyl-2H- pyrazol-3-yl)-2-{(4- pyridin-2-yl-benzyl)- [3-(4-trifluoromethyl- phenyl)-acryloyl]-amino}- propionic acid methyl ester | 61% | 3.22 | 549.40 |

*Analytic B

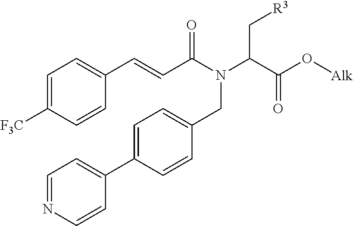

| | | | | LC-MS* | |
|---|---|---|---|---|---|
| | | | | t_{R} | |
| R³ | Alk | Chemical name | Yield | (min) | [M + H]⁺ |
| 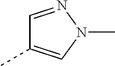 | Me | rac-3-(1-Methyl- 1H-pyrazol-4-yl)-2- {(4-pyridin-4-yl- benzyl)-[3-(4- trifluoromethyl- phenyl)- acryloyl]-amino}- propionic acid methyl ester | 72% | 2.69 | 549.40 |
| 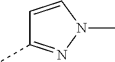 | Me | rac-3-(1-Methyl- 1H-pyrazol-3-yl)-2- {(4-pyridin-4-yl- benzyl)-[3-(4- trifluoromethyl- phenyl)- acryloyl]-amino}- propionic acid methyl ester | 67% | 2.74 | 549.30 |
| 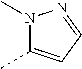 | Me | rac-3-(2-Methyl- 2H-pyrazol-3-yl)-2- {(4-pyridin-4-yl- benzyl)-[3-(4- trifluoromethyl- phenyl)- acryloyl]-amino}- propionic acid methyl ester | 61% | 2.74 | 549.30 |
| 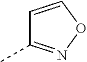 | Et | rac-3-Isoxazol-3-yl- 2-{(4-pyridin-4-yl- benzyl)-[3-(4- trifluoromethyl- phenyl)- acryloyl]-amino}- propionic acid ethyl ester | 56% | 2.91 | 550.40 |
| 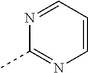 | Et | rac-2-{(4-Pyridin- 4-yl-benzyl)-[3-(4- trifluoromethyl- phenyl)- acryloyl]-amino}- 3-pyrimidin-2-yl- propionic acid ethyl ester | 78% | 2.78 | 561.40 |

*Analytic B

### Step 3

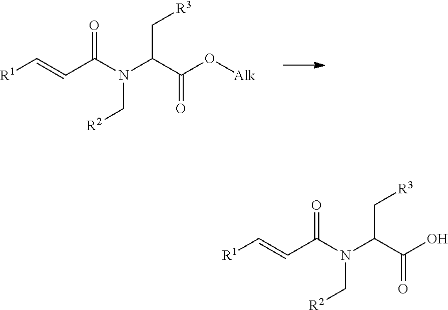

To a solution of the ester (1 eq) in MeOH (for methyl ester) or EtOH (for ethyl ester) (15 mL/mmol) was added dropwise aq. 2N NaOH (3.5 eq). The reaction mixture was stirred at rt for 1-14 h, then water was added and the solvent was removed in vacuo. The residue was acidified with aq. 1N HCl until pH <6. Solid NaCl was added until the aq. phase was saturated and then extracted with EA or DCM. The combined organic extracts were dried (MgSO₄), filtered and concentrated to afford the acid.

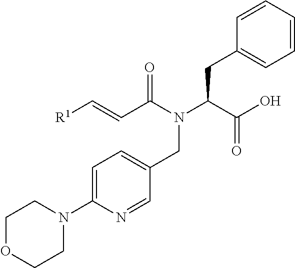

| | | | LC-MS | | |
|---|---|---|---|---|---|
| | | | t_{R} | | |
| R¹ | Chemical name | Yield | (min) | [M + H]⁺ | conditions |
| 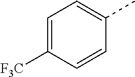 | (S)-2-{(6-Morpholin-4-yl-pyridin- 3-ylmethyl)-3-(4-trifluoromethyl- phenyl)-acryloyl]-amino}-3- phenyl-propionic acid | 93% | 0.61 | 539.95 | analytic A Zorbax Extend basic |
| 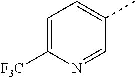 | (S)-2-{(6-Morpholin-4-yl-pyridin- 3-ylmethyl)-[3-(6-trifluoromethyl- pyridin-3-yl)-acryloyl]-amino}-3- phenyl-propionic acid | 97% | 0.83 | 541.03 | analytic A Zorbax SB-AQ acidic |
| 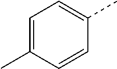 | (S)-2-[(6-Morpholin-4-yl-pyridin- 3-ylmethyl)-(3-p-tolyl-acryloyl)- amino]-3-phenyl-propionic acid | crude | 0.85 | 486.15 | analytic A Zorbax SB-AQ acidic |
| 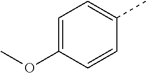 | (S)-2-[[3-(4-Methoxy-phenyl)- acryloyl]-(6-morpholin-4-yl- pyridin-3-ylmethyl)-amino]-3- phenyl-propionic acid | 99% | 0.83 | 502.15 | analytic A Zorbax SB-AQ acidic |

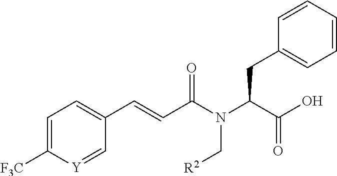

| | | | | LC-MS* | |
|---|---|---|---|---|---|
| | | | | t_{R} | |
| R² | Y | Chemical name | Yield | (min) | [M + H]⁺ |
| 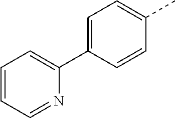 | CH | (S)-3-Phenyl-2-{(4-pyridin-2-yl- benzyl)-[3-(4-trifluoromethyl- phenyl)-acryloyl]-amino}- propionic acid | quant. | 0.91 | 531.25 |
| 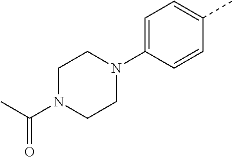 | CH | (S)-2-{[4-(4-Acetyl-piperazin-1- yl)-benzyl]-[3-(4- trifluoromethyl-phenyl)- acryloyl]-amino}-3-phenyl- propionic acid | 95% | 1.04 | 580.00 |
| 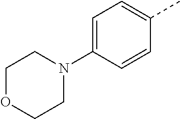 | CH | (S)-2-{(4-Morpholin-4-yl- benzyl)-[3-(4-trifluoromethyl- phenyl)-acryloyl]-amino}-3- phenyl-propionic acid | 89% | 1.04 | 539.13 |
| 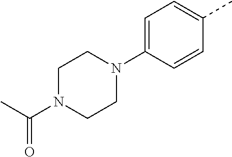 | N | (S)-2-{[4-(4-Acetyl-piperazin-1- yl)-benzyl]-[3-(6- trifluoromethyl-pyridin-3-yl)- acryloyl]-amino}-3-phenyl- propionic acid | quant. | 0.98 | 581.05 |
| 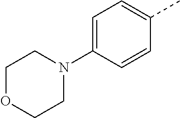 | N | (S)-2-{(4-Morpholin-4-yl- benzyl)-[3-(6-trifluoromethyl- pyridin-3-yl)-acryloyl]-amino}- 3-phenyl-propionic acid | 95% | 0.99 | 539.94 |

*Analytic A, Zorbax SB-AQ column, acidic conditions

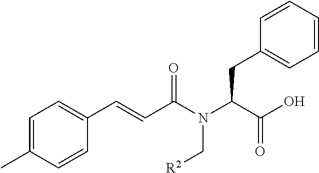

| | | | LC-MS* | |
|---|---|---|---|---|
| | | | t_{R} | |
| R² | Chemical name | Yield | (min) | [M + H]⁺ |
| 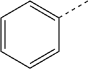 | (S)-2-[Benzyl-(3-p- tolyl- acryloyl)-amino]-3- phenyl-propionic acid | 99% | 1.07 | 400.06 |
| 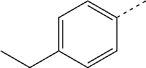 | (S)-2-[(4-Ethyl- benzyl)- (3-p-tolyl-acryloyl)- amino]-3-phenyl- propionic acid | 99% | 1.11 | 428.10 |
| 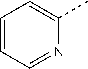 | (S)-3-Phenyl-2- [pyridin-2-ylmethyl- (3-p-tolyl-acryloyl)- amino]-propionic acid | 81% | 0.94 | 401.07 |

*Analytic A, Zorbax SB-AQ column, acidic conditions

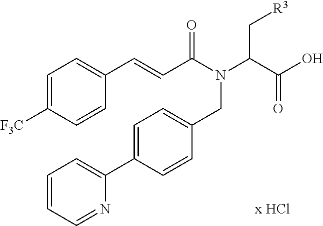

| | | | LC-MS* | |
|---|---|---|---|---|
| R³ | Chemical name | Yield | t_{R} (min) | [M + H]⁺ |
| 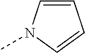 | (S)-2-{(4-Pyridin-2-yl-benzyl)-[3-(4- trifluoromethyl-phenyl)-acryloyl]-amino}-3- pyrrol-1-yl-propionic acid, hydrochloride salt | quant. | 3.41 | 520.40 |
| 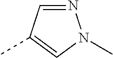 | rac-3-(1-Methyl-1H-pyrazol-4-yl)-2-{(4-pyridin- 2-yl-benzyl)-[3-(4-trifluoromethyl-phenyl)- acryloyl]-amino}-propionic acid, dihydrochloride salt | 90% | 2.90 | 535.50 |
| 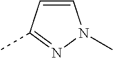 | rac-3-(1-Methyl-1H-pyrazol-3-yl)-2-{(4-pyridin- 2-yl-benzyl)-[3-(4-trifluoromethyl-phenyl)- acryloyl]-amino}-propionic acid, dihydrochloride salt | 85% | 2.95 | 535.40 |
| 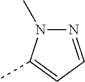 | rac-3-(2-Methyl-2H-pyrazol-3-yl)-2-{(4-pyridin- 2-yl-benzyl)-[3-(4-trifluoromethyl-phenyl)- acryloyl]-amino}-propionic acid, dihydrochloride salt | crude | 2.93 | 535.50 |

*Analytic B

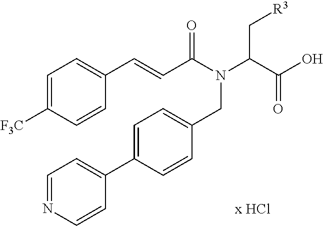

| | | | LC-MS* | |
|---|---|---|---|---|
| R³ | Chemical name | Yield | t_{R} (min) | [M + H]⁺ |
| 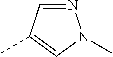 | rac-3-(1-Methyl-1H-pyrazol-4-yl)-2-{(4-pyridin- 4-yl-benzyl)-[3-(4-trifluoromethyl-phenyl)- acryloyl]-amino}-propionic acid, dihydrochloride salt | crude | 2.53 | 535.50 |
| 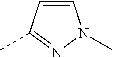 | rac-3-(1-Methyl-1H-pyrazol-3-yl)-2-{(4-pyridin- 4-yl-benzyl)-[3-(4-trifluoromethyl-phenyl)- acryloyl]-amino}-propionic acid, dihydrochloride salt | crude | 2.57 | 535.50 |
| 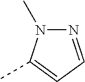 | rac-3-(2-Methyl-2H-pyrazol-3-yl)-2-{(4-pyridin- 4-yl-benzyl)-[3-(4-trifluoromethyl-phenyl)- acryloyl]-amino}-propionic acid, dihydrochloride | crude | 2.57 | 535.50 |
| 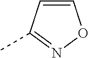 | rac-3-Isoxazol-3-yl-2-{(4-pyridin-4-yl-benzyl)- [3-(4-trifluoromethyl-phenyl)-acryloyl]-amino}- propionic acid, hydrochloride salt | 86% | 2.65 | 522.20 |
| 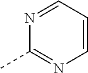 | rac-2-{(4-Pyridin-4-yl-benzyl)-[3-(4- trifluoromethyl-phenyl)-acryloyl]-amino}-3- pyrimidin-2-yl-propionic acid, hydrochloride salt | 67% | 2.51 | 533.50 |

*Analytic B

### Step 4

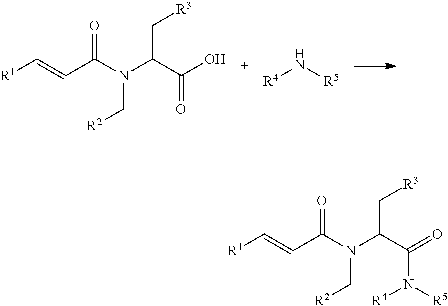

### General Procedure 1

A mixture of the acid (1 eq), TBTU (1.1 eq), and DIPEA (5 eq) in dry DMF (5 mL/mmol) was stirred at rt for 30 min. Then a solution of the amine NHR⁴R⁵ (1.05 eq) in dry DMF (5 mL/mmol) was added and the reaction mixture was stirred at rt overnight, then directly purified by preparative HPLC to afford the pure final compound.

### General Procedure 2

To an ice-cooled solution of the acid (1 eq) and the amine NHR⁴R⁵ (1.1 eq) in dry DCM (5 mL/mmol) were added successively a solution of PyBrop (1.1 eq) in dry DCM (5 mL/mmol) and DIPEA (2 eq). The reaction mixture was stirred at rt for 30 min, then the solvent was removed in vacuo and the crude product purified by preparative HPLC to afford the pure final compound.

| | | LC-MS (analytic A) | | |
|---|---|---|---|---|
| Ex. | | t_{R} | | |
| No. | Chemical name | (min) | [M + H]⁺ | HPLC |
| 136 | (S)-N-[1-Benzyl-2-(4-dimethylamino-piperidin-1-yl)-2- | 0.80 | 649.92 | Zorbax |
| | oxo-ethyl]-N-(6-morpholin-4-yl-pyridin-3-ylmethyl)-3-(4- | | | SB-AQ |
| | trifluoromethyl-phenyl)-acrylamide | | | acidic |
| 137 | rac-N-[2-(4-Dimethylamino-piperidin-1-yl)-1-(1-methyl- | 0.88 | 645.15 | XBridge |
| | 1H-pyrazol-4-ylmethyl)-2-oxo-ethyl]-N-(4-pyridin-2-yl- | | | basic |
| | benzyl)-3-(4-trifluoromethyl-phenyl)-acrylamide | | | |
| 138 | rac-N-[2-(4-Dimethylamino-piperidin-1-yl)-1-(1-methyl- | 0.89 | 645.14 | XBridge |
| | 1H-pyrazol-3-ylmethyl)-2-oxo-ethyl]-N-(4-pyridin-2-yl- | | | basic |
| | benzyl)-3-(4-trifluoromethyl-phenyl)-acrylamide | | | |
| 139 | rac-N-[2-(4-Dimethylamino-piperidin-1-yl)-1-(1-methyl- | 0.84 | 645.16 | XBridge |
| | 1H-pyrazol-4-ylmethyl)-2-oxo-ethyl]-N-(4-pyridin-4-yl- | | | basic |
| | benzyl)-3-(4-trifluoromethyl-phenyl)-acrylamide | | | |
| 140 | rac-N-[2-(4-Dimethylamino-piperidin-1-yl)-1-(1-methyl- | 0.85 | 645.11 | XBridge |
| | 1H-pyrazol-3-ylmethyl)-2-oxo-ethyl]-N-(4-pyridin-4-yl- | | | basic |
| | benzyl)-3-(4-trifluoromethyl-phenyl)-acrylamide | | | |
| 141 | rac-N-[2-(4-Dimethylamino-piperidin-1-yl)-1-(2-methyl- | 0.85 | 645.13 | XBridge |
| | 2H-pyrazol-3-ylmethyl)-2-oxo-ethyl]-N-(4-pyridin-4-yl- | | | basic |
| | benzyl)-3-(4-trifluoromethyl-phenyl)-acrylamide | | | |
| 142 | rac-N-[2-(4-Dimethylamino-piperidin-1-yl)-1-(2-methyl- | 0.90 | 645.12 | XBridge |
| | 2H-pyrazol-3-ylmethyl)-2-oxo-ethyl]-N-(4-pyridin-2-yl- | | | basic |
| | benzyl)-3-(4-trifluoromethyl-phenyl)-acrylamide | | | |
| 143 | (S)-N-[1-Benzyl-2-(4-benzyl-piperidin-1-yl)-2-oxo- | 1.04 | 697.19 | Zorbax |
| | ethyl]-N-(6-morpholin-4-yl-pyridin-3-ylmethyl)-3-(4- | | | SB-AQ |
| | trifluoromethyl-phenyl)-acrylamide | | | acidic |
| 144 | (S)-N-[1-Benzyl-2-(4-benzyl-piperidin-1-yl)-2-oxo- | 1.01 | 698.16 | Zorbax |
| | ethyl]-N-(6-morpholin-4-yl-pyridin-3-ylmethyl)-3-(6- | | | SB-AQ |
| | trifluoromethyl-pyridin-3-yl)-acrylamide | | | acidic |
| 145 | (S)-N-[1-Benzyl-2-(4-benzyl-piperidin-1-yl)-2-oxo- | 1.00 | 659.16 | Zorbax |
| | ethyl]-3-(4-methoxy-phenyl)-N-(6-morpholin-4-yl- | | | SB-AQ |
| | pyridin-3-ylmethyl)-acrylamide | | | acidic |
| 146 | (S)-N-[1-Benzyl-2-(4-benzyl-piperidin-1-yl)-2-oxo- | 1.02 | 643.11 | Zorbax |
| | ethyl]-N-(6-morpholin-4-yl-pyridin-3-ylmethyl)-3-p-tolyl- | | | SB-AQ |
| | acrylamide | | | acidic |
| 147 | (S)-N-(1-Benzyl-2-morpholin-4-yl-2-oxo-ethyl)-N-(6- | 0.88 | 555.00 | Zorbax |
| | morpholin-4-yl-pyridin-3-ylmethyl)-3-p-tolyl-acrylamide | | | SB-AQ |
| | | | | acidic |
| 148 | (S)-N-(1-Benzyl-2-morpholin-4-yl-2-oxo-ethyl)-3-(4- | 0.85 | 570.98 | Zorbax |
| | methoxy-phenyl)-N-(6-morpholin-4-yl-pyridin-3- | | | SB-AQ |
| | ylmethyl)-acrylamide | | | acidic |
| 149 | (S)-N-[1-Benzyl-2-(5,8-dihydro-6H-[1,7]naphthyridin-7- | 0.88 | 647.23 | Zorbax |
| | yl)-2-oxo-ethyl]-N-(4-pyridin-2-yl-benzyl)-3-(4- | | | SB-AQ |
| | trifluoromethyl-phenyl)-acrylamide | | | acidic |
| 150 | (S)-N-[1-(2-Dibutylamino-ethylcarbamoyl)-2-phenyl- | 0.92 | 685.51 | Zorbax |
| | ethyl]-N-(4-pyridin-2-yl-benzyl)-3-(4-trifluoromethyl- | | | SB-AQ |
| | phenyl)-acrylamide | | | acidic |
| 151 | (S)-N-[1-(2-Morpholin-4-yl-ethylcarbamoyl)-2-phenyl- | 0.81 | 643.42 | Zorbax |
| | ethyl]-N-(4-pyridin-2-yl-benzyl)-3-(4-trifluoromethyl- | | | SB-AQ |
| | phenyl)-acrylamide | | | acidic |
| 152 | N-[1-(rac-1-Aza-bicyclo[2.2.2]oct-3-ylcarbamoyl)-(S)-2- | 0.80 | 639.40 | Zorbax |
| | phenyl-ethyl]-N-(4-pyridin-2-yl-benzyl)-3-(4- | | | SB-AQ |
| | trifluoromethyl-phenyl)-acrylamide | | | acidic |
| 153 | (S)-N-[2-Phenyl-1-(2-piperidin-1-yl-ethylcarbamoyl)- | 0.83 | 641.44 | Zorbax |
| | ethyl]-N-(4-pyridin-2-yl-benzyl)-3-(4-trifluoromethyl- | | | SB-AQ |
| | phenyl)-acrylamide | | | acidic |
| 154 | (S)-N-[1-(2-Azepan-1-yl-ethylcarbamoyl)-2-phenyl- | 0.86 | 655.43 | Zorbax |
| | ethyl]-N-(4-pyridin-2-yl-benzyl)-3-(4-trifluoromethyl- | | | SB-AQ |
| | phenyl)-acrylamide | | | acidic |
| 155 | (S)-N-[1-(2-Diisopropylamino-ethylcarbamoyl)-2- | 0.85 | 657.46 | Zorbax |
| | phenyl-ethyl]-N-(4-pyridin-2-yl-benzyl)-3-(4- | | | SB-AQ |
| | trifluoromethyl-phenyl)-acrylamide | | | acidic |
| 156 | (S)-N-[1-(2-Dimethylamino-ethylcarbamoyl)-2-phenyl- | 0.80 | 601.41 | Zorbax |
| | ethyl]-N-(4-pyridin-2-yl-benzyl)-3-(4-trifluoromethyl- | | | SB-AQ |
| | phenyl)-acrylamide | | | acidic |
| 157 | (S)-N-{1-[(2-Methoxy-ethyl)-methyl-carbamoyl]-2- | 0.92 | 610.93 | Zorbax |
| | phenyl-ethyl}-N-(6-morpholin-4-yl-pyridin-3-ylmethyl)- | | | SB-AQ |
| | 3-(4-trifluoromethyl-phenyl)-acrylamide | | | acidic |
| 158 | (S)-N-{1-[(2-Methoxy-ethyl)-methyl-carbamoyl]-2- | 0.90 | 557.01 | Zorbax |
| | phenyl-ethyl}-N-(6-morpholin-4-yl-pyridin-3-ylmethyl)- | | | SB-AQ |
| | 3-p-tolyl-acrylamide | | | acidic |
| 159 | (S)-N-{1-[(2-Methoxy-ethyl)-methyl-carbamoyl]-2- | 0.88 | 573.01 | Zorbax |
| | phenyl-ethyl}-3-(4-methoxy-phenyl)-N-(6-morpholin-4- | | | SB-AQ |
| | yl-pyridin-3-ylmethyl)-acrylamide | | | acidic |
| 160 | (S)-N-Benzyl-N-{1-[(2-methoxy-ethyl)-methyl- | 1.12 | 471.63 | Zorbax |
| | carbamoyl]-2-phenyl-ethyl}-3-p-tolyl-acrylamide | | | SB-AQ |
| | | | | acidic |
| 161 | (S)-N-(4-Ethyl-benzyl)-N-{1-[(2-methoxy-ethyl)-methyl- | 1.16 | 499.70 | Zorbax |
| | carbamoyl]-2-phenyl-ethyl}-3-p-tolyl-acrylamide | | | SB-AQ |
| | | | | acidic |
| 162 | (S)-N-{1-[(2-Methoxy-ethyl)-methyl-carbamoyl]-2- | 0.90 | 472.68 | Zorbax |
| | phenyl-ethyl}-N-pyridin-2-ylmethyl-3-p-tolyl-acrylamide, | | | SB-AQ |
| | formic acid | | | acidic |
| 163 | (S)-N-{1-[(2-Methoxy-ethyl)-methyl-carbamoyl]-2- | 0.99 | 591.10 | XBridge |
| | pyrrol-1-yl-ethyl}-N-(4-pyridin-2-yl-benzyl)-3-(4- | | | basic |
| | trifluoromethyl-phenyl)-acrylamide | | | |
| 164 | rac-N-[1-[(2-Methoxy-ethyl)-methyl-carbamoyl]-2-(1- | 0.90 | 606.11 | XBridge |
| | methyl-1H-pyrazol-4-yl)-ethyl]-N-(4-pyridin-2-yl- | | | basic |
| | benzyl)-3-(4-trifluoromethyl-phenyl)-acrylamide | | | |
| 165 | rac-N-[1-[(2-Methoxy-ethyl)-methyl-carbamoyl]-2-(1- | 0.91 | 606.10 | XBridge |
| | methyl-1H-pyrazol-3-yl)-ethyl]-N-(4-pyridin-2-yl- | | | basic |
| | benzyl)-3-(4-trifluoromethyl-phenyl)-acrylamide | | | |
| 166 | rac-N-[1-[(2-Methoxy-ethyl)-methyl-carbamoyl]-2-(1- | 0.86 | 606.08 | XBridge |
| | methyl-1H-pyrazol-4-yl)-ethyl]-N-(4-pyridin-4-yl- | | | basic |
| | benzyl)-3-(4-trifluoromethyl-phenyl)-acrylamide | | | |
| 167 | rac-N-[1-[(2-Methoxy-ethyl)-methyl-carbamoyl]-2-(1- | 0.87 | 606.07 | XBridge |
| | methyl-1H-pyrazol-3-yl)-ethyl]-N-(4-pyridin-4-yl- | | | basic |
| | benzyl)-3-(4-trifluoromethyl-phenyl)-acrylamide | | | |
| 168 | rac-N-[1-[(2-Methoxy-ethyl)-methyl-carbamoyl]-2-(2- | 0.87 | 606.07 | XBridge |
| | methyl-2H-pyrazol-3-yl)-ethyl]-N-(4-pyridin-4-yl- | | | basic |
| | benzyl)-3-(4-trifluoromethyl-phenyl)-acrylamide | | | |
| 169 | rac-N-{2-Isoxazol-3-yl-1-[(2-methoxy-ethyl)-methyl- | 0.90 | 592.98 | XBridge |
| | carbamoyl]-ethyl}-N-(4-pyridin-4-yl-benzyl)-3-(4- | | | basic |
| | trifluoromethyl-phenyl)-acrylamide | | | |
| 170 | rac-N-{1-[(2-Methoxy-ethyl)-methyl-carbamoyl]-2- | 0.86 | 604.03 | XBridge |
| | pyrimidin-2-yl-ethyl}-N-(4-pyridin-4-yl-benzyl)-3-(4- | | | basic |
| | trifluoromethyl-phenyl)-acrylamide | | | |
| 171 | (S)-N-{1-[(2-Dimethylamino-ethyl)-methyl-carbamoyl]- | 0.82 | 615.52 | Zorbax |
| | 2-phenyl-ethyl}-N-(4-pyridin-2-yl-benzyl)-3-(4- | | | SB-AQ |
| | trifluoromethyl-phenyl)-acrylamide | | | acidic |
| 172 | N-{1-[((S)-1-Benzyl-pyrrolidin-3-yl)-methyl-carbamoyl]- | 0.90 | 703.52 | Zorbax |
| | (S)-2-phenyl-ethyl}-N-(4-pyridin-2-yl-benzyl)-3-(4- | | | SB-AQ |
| | trifluoromethyl-phenyl)-acrylamide | | | acidic |
| 173 | N-{1-[((R)-1-Benzyl-pyrrolidin-3-yl)-methyl-carbamoyl]- | 0.89 | 703.40 | Zorbax |
| | (S)-2-phenyl-ethyl}-N-(4-pyridin-2-yl-benzyl)-3-(4- | | | SB-AQ |
| | trifluoromethyl-phenyl)-acrylamide | | | acidic |
| 174 | (S)-N-{1-[Benzyl-(2-dimethylamino-ethyl)-carbamoyl]- | 0.90 | 691.43 | Zorbax |
| | 2-phenyl-ethyl}-N-(4-pyridin-2-yl-benzyl)-3-(4- | | | SB-AQ |
| | trifluoromethyl-phenyl)-acrylamide | | | acidic |
| 175 | (S)-N-{1-[(2-Hydroxy-ethyl)-methyl-carbamoyl]-2- | 1.00 | 596.17 | Zorbax |
| | phenyl-ethyl}-N-(4-morpholin-4-yl-benzyl)-3-(4- | | | SB-AQ |
| | trifluoromethyl-phenyl)-acrylamide | | | acidic |
| 176 | (S)-N-[4-(4-Acetyl-piperazin-1-yl)-benzyl]-N-{1-[(2- | 1.00 | 637.28 | Zorbax |
| | hydroxy-ethyl)-methyl-carbamoyl]-2-phenyl-ethyl}-3-(4- | | | SB-AQ |
| | trifluoromethyl-phenyl)-acrylamide | | | acidic |
| 177 | (S)-N-{1-[(4-Methoxy-benzyl)-methyl-carbamoyl]-2- | 1.11 | 672.46 | Zorbax |
| | phenyl-ethyl}-N-(4-morpholin-4-yl-benzyl)-3-(6- | | | SB-AQ |
| | trifluoromethyl-pyridin-3-yl)-acrylamide | | | acidic |

### Preparation of Compounds of Formula I Via Method C:

### Step 1

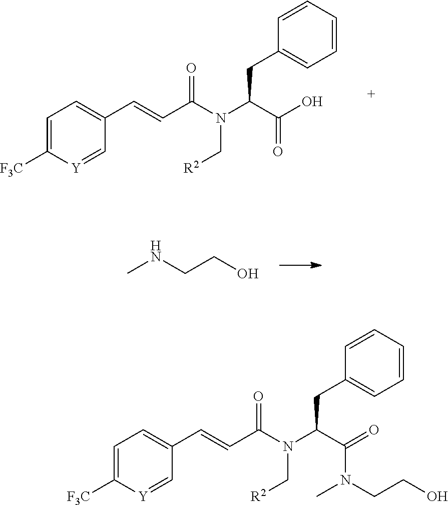

To a mixture of the acid (1 eq) and TBTU (2 eq) in dry DCM (25 mL/mmol) was added DIPEA (3 eq). The resulting mixture was stirred at rt for 15 min and then 2-(methylamino)ethanol (2 eq) was added. The reaction mixture was stirred at rt overnight under nitrogen atmosphere, then concentrated in vacuo. The resulting residue was taken up in a mixture of EA and a sat. NH₄Cl solution. The organic layer was separated and washed 4 times with sat. NH₄Cl. The combined aq. phases were extracted twice with EA. The combined organic layers were washed with brine, dried (MgSO₄), filtered and concentrated under reduced pressure. FC (n-heptane/EA/MeOH or DCM/MeOH system) afforded the pure amide.

| | | | | LC-MS* | |
|---|---|---|---|---|---|
| R² | Y | Chemical name | Yield | t_{R} (min) | [M + H]⁺ |
| 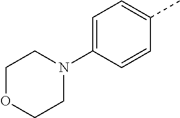 | CH | (S)-N-{1-[(2-Hydroxy-ethyl)- methyl-carbamoyl]-2-phenyl- ethyl}-N-(4-morpholin-4-yl- benzyl)-3-(4-trifluoromethyl- phenyl)-acrylamide | 91% | 1.00 | 596.17 |
| 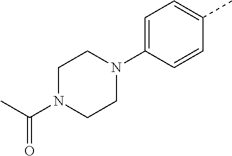 | CH | (S)-N-[4-(4-Acetyl-piperazin-1-yl)- benzyl]-N-{1-[(2-hydroxy-ethyl)- methyl-carbamoyl]-2-phenyl- ethyl}-3-(4-trifluoromethyl-phenyl)- acrylamide | 64% | 1.00 | 637.28 |
| 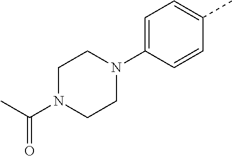 | N | (S)-N-[4-(4-Acetyl-piperazin-1-yl)- benzyl]-N-{1-[(2-hydroxy-ethyl)- methyl-carbamoyl]-2-phenyl- ethyl}-3-(6-trifluoromethyl-pyridin- 3-yl)-acrylamide | 66% | 0.93 | 638.09 |

*Analytic A, Zorbax SB-AQ column, acidic conditions

### Step 2

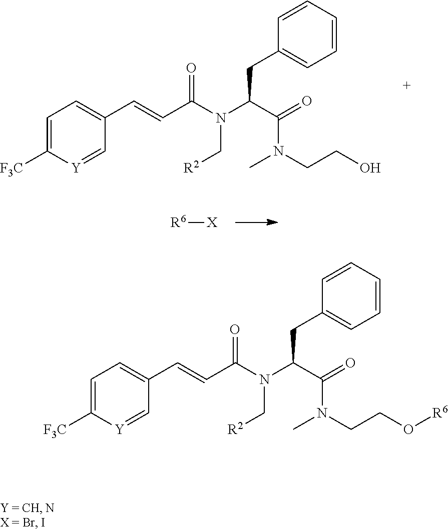

To a stirred solution of the alcohol (1 eq) in dry THF (5 ml/mmol) was added NaH (1.5 eq). Then was added the halide R⁶X (1 eq) in the resulting orange mixture. The reaction mixture was stirred at rt overnight, then quenched with a small amount of water. The solvent was removed in vacuo and the crude product purified by preparative HPLC to afford the pure final compound.

| Example | | LC-MS* | |
|---|---|---|---|
| number | Chemical name | t_{R} (min) | [M + H]⁺ |
| 178 | (S)-N-[4-(4-Acetyl-piperazin-1-yl)-benzyl]-N-{1-[(2- | 1.07 | 651.24 |
| | methoxy-ethyl)-methyl-carbamoyl]-2-phenyl-ethyl}-3-(4- | | |
| | trifluoromethyl-phenyl)-acrylamide | | |
| 179 | (S)-N-{1-[(2-Methoxy-ethyl)-methyl-carbamoyl]-2-phenyl- | 1.07 | 610.27 |
| | ethyl}-N-(4-morpholin-4-yl-benzyl)-3-(4-trifluoromethyl- | | |
| | phenyl)-acrylamide, formic acid | | |
| 180 | (S)-N-[4-(4-Acetyl-piperazin-1-yl)-benzyl]-N-(1-{methyl-[2- | 1.03 | 715.27 |
| | (pyrimidin-2-yloxy)-ethyl]-carbamoyl}-2-phenyl-ethyl)-3-(4- | | |
| | trifluoromethyl-phenyl)-acrylamide | | |
| 181 | (S)-N-[4-(4-Acetyl-piperazin-1-yl)-benzyl]-N-{1-[(2- | 1.12 | 727.36 |
| | benzyloxy-ethyl)-methyl-carbamoyl]-2-phenyl-ethyl}-3-(4- | | |
| | trifluoromethyl-phenyl)-acrylamide | | |
| 182 | (S)-N-{1-[(2-Benzyloxy-ethyl)-methyl-carbamoyl]-2- | 1.14 | 686.25 |
| | phenyl-ethyl}-N-(4-morpholin-4-yl-benzyl)-3-(4- | | |
| | trifluoromethyl-phenyl)-acrylamide | | |
| 183 | (S)-N-[4-(4-Acetyl-piperazin-1-yl)-benzyl]-N-(1-{[2-(2,4- | 1.19 | 755.22 |
| | dimethyl-benzyloxy)-ethyl]-methyl-carbamoyl}-2-phenyl- | | |
| | ethyl)-3-(4-trifluoromethyl-phenyl)-acrylamide, formic acid | | |
| 184 | (S)-N-[4-(4-Acetyl-piperazin-1-yl)-benzyl]-N-(1-{[2-(3- | 1.15 | 775.13 |
| | fluoro-4-methoxy-benzyloxy)-ethyl]-methyl-carbamoyl}-2- | | |
| | phenyl-ethyl)-3-(4-trifluoromethyl-phenyl)-acrylamide, | | |
| | formic acid | | |
| 185 | (S)-N-[4-(4-Acetyl-piperazin-1-yl)-benzyl]-N-(1-{[2-(3- | 1.14 | 752.19 |
| | cyano-benzyloxy)-ethyl]-methyl-carbamoyl}-2-phenyl- | | |
| | ethyl)-3-(4-trifluoromethyl-phenyl)-acrylamide, formic acid | | |
| 186 | (S)-N-[4-(4-Acetyl-piperazin-1-yl)-benzyl]-N-(1-{methyl-[2- | 1.20 | 811.12 |
| | (3-trifluoromethoxy-benzyloxy)-ethyl]-carbamoyl}-2- | | |
| | phenyl-ethyl)-3-(4-trifluoromethyl-phenyl)-acrylamide, | | |
| | formic acid | | |
| 187 | (S)-N-[4-(4-Acetyl-piperazin-1-yl)-benzyl]-N-(1-{[2-(3- | 1.15 | 757.19 |
| | methoxy-benzyloxy)-ethyl]-methyl-carbamoyl}-2-phenyl- | | |
| | ethyl)-3-(4-trifluoromethyl-phenyl)-acrylamide, formic acid | | |
| 188 | (S)-N-[4-(4-Acetyl-piperazin-1-yl)-benzyl]-N-(1-{[2-(4- | 1.16 | 793.14 |
| | difluoromethoxy-benzyloxy)-ethyl]-methyl-carbamoyl}-2- | | |
| | phenyl-ethyl)-3-(4-trifluoromethyl-phenyl)-acrylamide, | | |
| | formic acid | | |
| 189 | (S)-N-[4-(4-Acetyl-piperazin-1-yl)-benzyl]-N-(1-{methyl-[2- | 1.10 | 782.20 |
| | (1-methyl-1H-benzotriazol-5-ylmethoxy)-ethyl]- | | |
| | carbamoyl}-2-phenyl-ethyl)-3-(4-trifluoromethyl-phenyl)- | | |
| | acrylamide, formic acid | | |
| 190 | (S)-N-[4-(4-Acetyl-piperazin-1-yl)-benzyl]-N-(1-{methyl-[2- | 0.93 | 728.20 |
| | (pyridin-3-ylmethoxy)-ethyl]-carbamoyl}-2-phenyl-ethyl)-3- | | |
| | (4-trifluoromethyl-phenyl)-acrylamide, formic acid | | |
| 191 | (S)-N-(1-{[2-(3-Methoxy-benzyloxy)-ethyl]-methyl- | 1.12 | 716.30 |
| | carbamoyl}-2-phenyl-ethyl)-N-(4-morpholin-4-yl-benzyl)-3- | | |
| | (4-trifluoromethyl-phenyl)-acrylamide, formic acid | | |
| 192 | (S)-N-{1-[(2-Ethoxy-ethyl)-methyl-carbamoyl]-2-phenyl- | 1.10 | 623.84 |
| | ethyl}-N-(4-morpholin-4-yl-benzyl)-3-(4-trifluoromethyl- | | |
| | phenyl)-acrylamide | | |
| 193 | (S)-N-[4-(4-Acetyl-piperazin-1-yl)-benzyl]-N-{1-[(2-ethoxy- | 1.10 | 664.99 |
| | ethyl)-methyl-carbamoyl]-2-phenyl-ethyl}-3-(4- | | |
| | trifluoromethyl-phenyl)-acrylamide | | |
| 194 | (S)-N-[4-(4-Acetyl-piperazin-1-yl)-benzyl]-N-(1-{[2-(2,4- | 1.20 | 754.94 |
| | dimethyl-benzyloxy)-ethyl]-methyl-carbamoyl}-2-phenyl- | | |
| | ethyl)-3-(4-trifluoromethyl-phenyl)-acrylamide | | |
| 195 | (S)-N-(1-{[2-(2,4-Dimethyl-benzyloxy)-ethyl]-methyl- | 1.19 | 714.01 |
| | carbamoyl}-2-phenyl-ethyl)-N-(4-morpholin-4-yl-benzyl)-3- | | |
| | (4-trifluoromethyl-phenyl)-acrylamide | | |
| 196 | (S)-N-(1-{[2-(3-Fluoro-4-methoxy-benzyloxy)-ethyl]- | 1.15 | 733.98 |
| | methyl-carbamoyl}-2-phenyl-ethyl)-N-(4-morpholin-4-yl- | | |
| | benzyl)-3-(4-trifluoromethyl-phenyl)-acrylamide | | |
| 197 | (S)-N-(1-{[2-(3-Cyano-benzyloxy)-ethyl]-methyl- | 1.14 | 710.98 |
| | carbamoyl}-2-phenyl-ethyl)-N-(4-morpholin-4-yl-benzyl)-3- | | |
| | (4-trifluoromethyl-phenyl)-acrylamide | | |
| 198 | (S)-N-(1-{Methyl-[2-(3-trifluoromethoxy-benzyloxy)-ethyl]- | 1.20 | 770.03 |
| | carbamoyl}-2-phenyl-ethyl)-N-(4-morpholin-4-yl-benzyl)-3- | | |
| | (4-trifluoromethyl-phenyl)-acrylamide | | |
| 199 | (S)-N-(1-{[2-(3,5-Dimethoxy-benzyloxy)-ethyl]-methyl- | 1.15 | 745.99 |
| | carbamoyl}-2-phenyl-ethyl)-N-(4-morpholin-4-yl-benzyl)-3- | | |
| | (4-trifluoromethyl-phenyl)-acrylamide | | |
| 200 | (S)-N-(1-{[2-(3-Methoxy-benzyloxy)-ethyl]-methyl- | 1.15 | 716.03 |
| | carbamoyl}-2-phenyl-ethyl)-N-(4-morpholin-4-yl-benzyl)-3- | | |
| | (4-trifluoromethyl-phenyl)-acrylamide | | |
| 201 | (S)-N-(1-{[2-(4-Difluoromethoxy-benzyloxy)-ethyl]-methyl- | 1.16 | 751.93 |
| | carbamoyl}-2-phenyl-ethyl)-N-(4-morpholin-4-yl-benzyl)-3- | | |
| | (4-trifluoromethyl-phenyl)-acrylamide | | |
| 202 | (S)-N-(1-{Methyl-[2-(1-methyl-1H-benzotriazol-5- | 1.10 | 741.08 |
| | ylmethoxy)-ethyl]-carbamoyl}-2-phenyl-ethyl)-N-(4- | | |
| | morpholin-4-yl-benzyl)-3-(4-trifluoromethyl-phenyl)- | | |
| | acrylamide | | |
| 203 | (S)-N-(1-{Methyl-[2-(pyridin-3-ylmethoxy)-ethyl]- | 0.93 | 686.98 |
| | carbamoyl}-2-phenyl-ethyl)-N-(4-morpholin-4-yl-benzyl)-3- | | |
| | (4-trifluoromethyl-phenyl)-acrylamide | | |
| 204 | (S)-N-[4-(4-Acetyl-piperazin-1-yl)-benzyl]-N-(1-{methyl-[2- | 1.10 | 731.97 |
| | (5-methyl-isoxazol-3-ylmethoxy)-ethyl]-carbamoyl}-2- | | |
| | phenyl-ethyl)-3-(4-trifluoromethyl-phenyl)-acrylamide | | |
| 205 | (S)-N-[4-(4-Acetyl-piperazin-1-yl)-benzyl]-N-(1-{methyl-[2- | 0.92 | 727.99 |
| | (pyridin-4-ylmethoxy)-ethyl]-carbamoyl}-2-phenyl-ethyl)-3- | | |
| | (4-trifluoromethyl-phenyl)-acrylamide | | |
| 206 | (S)-N-[4-(4-Acetyl-piperazin-1-yl)-benzyl]-N-(1-{methyl-[2- | 1.18 | 784.87 |
| | (5-trifluoromethyl-furan-2-ylmethoxy)-ethyl]-carbamoyl}-2- | | |
| | phenyl-ethyl)-3-(4-trifluoromethyl-phenyl)-acrylamide | | |
| 207 | (S)-N-[4-(4-Acetyl-piperazin-1-yl)-benzyl]-N-{1-[(2- | 1.13 | 691.03 |
| | cyclopropylmethoxy-ethyl)-methyl-carbamoyl]-2-phenyl- | | |
| | ethyl}-3-(4-trifluoromethyl-phenyl)-acrylamide | | |
| 208 | (S)-N-[4-(4-Acetyl-piperazin-1-yl)-benzyl]-N-(1-{methyl-[2- | 0.87 | 728.97 |
| | (pyridin-4-ylmethoxy)-ethyl]-carbamoyl}-2-phenyl-ethyl)-3- | | |
| | (6-trifluoromethyl-pyridin-3-yl)-acrylamide | | |
| 209 | (S)-N-[4-(4-Acetyl-piperazin-1-yl)-benzyl]-N-(1-{methyl-[2- | 1.05 | 732.97 |
| | (5-methyl-isoxazol-3-ylmethoxy)-ethyl]-carbamoyl}-2- | | |
| | phenyl-ethyl)-3-(6-trifluoromethyl-pyridin-3-yl)-acrylamide | | |
| 210 | (S)-N-[4-(4-Acetyl-piperazin-1-yl)-benzyl]-N-(1-{[2-(2- | 1.16 | 770.83 |
| | benzyloxy-ethoxy)-ethyl]-methyl-carbamoyl}-2-phenyl- | | |
| | ethyl)-3-(4-trifluoromethyl-phenyl)-acrylamide | | |
| 211 | (S)-N-[4-(4-Acetyl-piperazin-1-yl)-benzyl]-N-{1-[(2- | 1.08 | 676.00 |
| | cyanomethoxy-ethyl)-methyl-carbamoyl]-2-phenyl-ethyl}- | | |
| | 3-(4-trifluoromethyl-phenyl)-acrylamide | | |
| 212 | (S)-N-[4-(4-Acetyl-piperazin-1-yl)-benzyl]-N-{1-[(2-allyloxy- | 1.12 | 677.10 |
| | ethyl)-methyl-carbamoyl]-2-phenyl-ethyl}-3-(4- | | |
| | trifluoromethyl-phenyl)-acrylamide | | |
| 213 | (S)-N-[4-(4-Acetyl-piperazin-1-yl)-benzyl]-N-{1-[(2- | 1.00 | 693.99 |
| | carbamoylmethoxy-ethyl)-methyl-carbamoyl]-2-phenyl- | | |
| | ethyl}-3-(4-trifluoromethyl-phenyl)-acrylamide | | |
| 214 | (S)-N-[4-(4-Acetyl-piperazin-1-yl)-benzyl]-N-(1-{methyl-[2- | 0.94 | 728.00 |
| | (pyridin-2-ylmethoxy)-ethyl]-carbamoyl}-2-phenyl-ethyl)-3- | | |
| | (4-trifluoromethyl-phenyl)-acrylamide | | |
| 215 | (S)-N-[4-(4-Acetyl-piperazin-1-yl)-benzyl]-N-(1-{methyl-[2- | 0.89 | 728.98 |
| | (pyridin-2-ylmethoxy)-ethyl]-carbamoyl}-2-phenyl-ethyl)-3- | | |
| | (6-trifluoromethyl-pyridin-3-yl)-acrylamide | | |
| 216 | (S)-N-[4-(4-Acetyl-piperazin-1-yl)-benzyl]-N-(1-{[2-(2- | 1.12 | 771.89 |
| | benzyloxy-ethoxy)-ethyl]-methyl-carbamoyl}-2-phenyl- | | |
| | ethyl)-3-(6-trifluoromethyl-pyridin-3-yl)-acrylamide | | |
| 217 | (S)-N-[4-(4-Acetyl-piperazin-1-yl)-benzyl]-N-{1-[(2- | 1.02 | 677.00 |
| | cyanomethoxy-ethyl)-methyl-carbamoyl]-2-phenyl-ethyl}- | | |
| | 3-(6-trifluoromethyl-pyridin-3-yl)-acrylamide | | |
| 218 | (S)-N-[4-(4-Acetyl-piperazin-1-yl)-benzyl]-N-{1-[(2-allyloxy- | 1.07 | 677.97 |
| | ethyl)-methyl-carbamoyl]-2-phenyl-ethyl}-3-(6- | | |
| | trifluoromethyl-pyridin-3-yl)-acrylamide | | |

*Analytic A, Zorbax SB-AQ column, acidic conditions

### Preparation of Compounds of Formula I Via Method D:

### Preparation of Methyl-(2-methylamino-ethyl)-carbamic acid tert-butyl ester

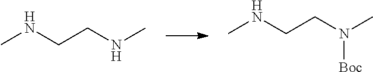

To an ice-cooled solution of N,N′-dimethyethylenediamine (10 mL, 91.0 mmol) in dry THF (150 mL) was added a solution of Boc₂O (4.97 g, 22.8 mmol) in dry THF (50 mL) over 30 minutes. The reaction mixture was stirred for 1 h at 0° C. then at rt overnight, and concentrated in vacuo. The resulting residue was taken up in a mixture of EA and a sat. NH₄Cl solution. The organic layer was separated, washed with brine, dried (MgSO₄), filtered and concentrated under reduced pressure. FC (10% MeOH in DCM) afforded the title compound as a yellow oil (2.90 g, 17%).

LC-MS (analytic A, Zorbax SB-AQ column, acidic conditions): t_{R}=0.50 min; [M+H]⁺=189.40.

### Step 1

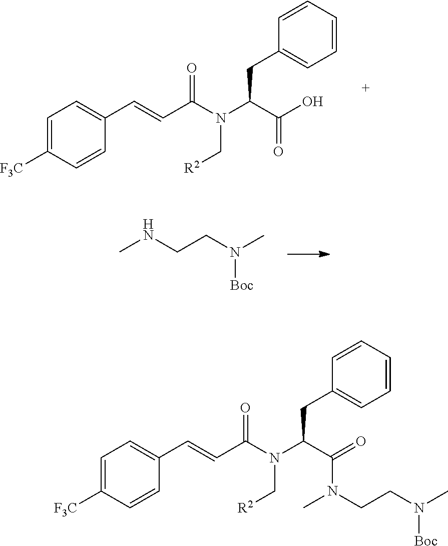

To a mixture of the acid (1 eq), TBTU (2 eq), and cat. DMAP in dry DCM (25 mL/mmol) was added DIPEA (3 eq). The resulting mixture was stirred at rt for 15 min and then methyl-(2-methylamino-ethyl)-carbamic acid tert-butyl ester (1 eq) was added. The reaction mixture was stirred at rt overnight under nitrogen atmosphere, then concentrated in vacuo. The resulting residue was taken up in a mixture of EA and a sat. NH₄Cl solution. The organic layer was separated and washed 4 times with sat. NH₄Cl. The combined aq. phases were extracted twice with EA. The combined organic layers were washed with brine, dried (MgSO₄), filtered and concentrated under reduced pressure. FC (n-heptane/EA or EA/MeOH system) afforded the pure amide.

| | | | LC-MS* | |
|---|---|---|---|---|
| R² | Chemical name | Yield | t_{R} (min) | [M + H]⁺ |
| 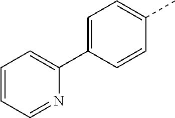 | (S)-Methyl-{2-[methyl-(3-phenyl-2-{(4- pyridin-2-yl-benzyl)-[3-(4- trifluoromethyl-phenyl)-acryloyl]-amino}- propionyl)-amino]-ethyl}-carbamic acid tert-butyl ester | 77% | 0.98 | 701.74 |
| 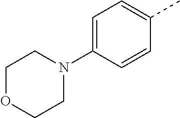 | (S)-Methyl-{2-[methyl-(2-{(4-morpholin- 4-yl-benzyl)-[3-(4-trifluoromethyl- phenyl)-acryloyl]-amino}-3-phenyl- propionyl)-amino]-ethyl}-carbamic acid tert-butyl ester | 74% | 1.14 | 709.27 |
| 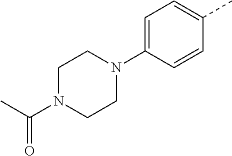 | (S)-{2-[(2-{[4-(4-Acetyl-piperazin-1-yl)- benzyl]-[3-(4-trifluoromethyl-phenyl)- acryloyl]-amino}-3-phenyl-propionyl)- methyl-amino]-ethyl}-methyl-carbamic acid tert-butyl ester | 69% | 1.14 | 750.29 |

*Analytic A, Zorbax SB-AQ column, acidic conditions

### Step 2

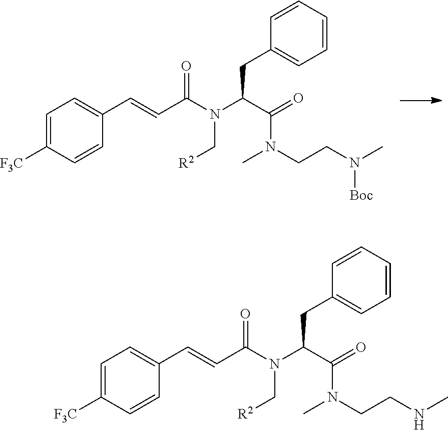

To an ice-cooled solution of the Boc-protected amine (1 eq) in dry DCM (10 mL/mmol) was added dropwise TFA (10 eq). The resulting reaction mixture was stirred at 0° C. for 30 min, then at rt for 5 h under nitrogen atmosphere and then concentrated in vacuo. The resulting residue was dissolved in EA and washed with a 2N NaOH solution. The organic extract was dried (MgSO₄), filtered and concentrated under reduced pressure. FC (DCM/MeOH/NH₄OH system) afforded the free secondary amine.

| | | | LC-MS* | |
|---|---|---|---|---|
| R² | Chemical name | Yield | t_{R} (min) | [M + H]⁺ |
| 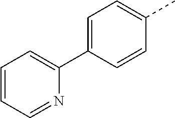 | (S)-N-{1-[Methyl-(2-methylamino-ethyl)- carbamoyl]-2-phenyl-ethyl}-N-(4-pyridin- 2-yl-benzyl)-3-(4-trifluoromethyl- phenyl)-acrylamide | 70% | 0.80 | 601.64 |
| 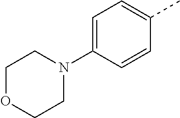 | (S)-N-{1-[Methyl-(2-methylamino-ethyl)- carbamoyl]-2-phenyl-ethyl}-N-(4- morpholin-4-yl-benzyl)-3-(4- trifluoromethyl-phenyl)-acrylamide | 58% | 0.91 | 609.09 |
| 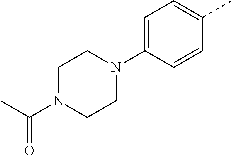 | (S)-N-[4-(4-Acetyl-piperazin-1-yl)- benzyl]-N-{1-[methyl-(2-methylamino- ethyl)-carbamoyl]-2-phenyl-ethyl}-3-(4- trifluoromethyl-phenyl)-acrylamide | 61% | 0.90 | 650.32 |

*Analytic A, Zorbax SB-AQ column, acidic conditions

### Step 3

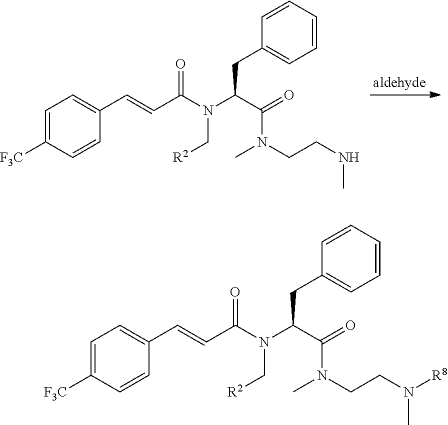

A mixture of the amine (1 eq), the aldehyde (2 eq), sodium triacetoxyborohydride (2.5 eq), and acetic acid (2 eq) in dry THF or MeCN (10 ml/mmol) was stirred at rt overnight, then directly purified by preparative HPLC to afford the pure final compound.

| | | LC-MS* | |
|---|---|---|---|
| | | t_{R} | |
| Ex. No. | Chemical name | (min) | [M + H]⁺ |
| 219 | (S)-N-[1-({2-[(5-Bromo-furan-2-ylmethyl)-methyl-amino]- | 0.89 | 761.68 |
| | ethyl}-methyl-carbamoyl)-2-phenyl-ethyl]-N-(4-pyridin-2-yl- | | |
| | benzyl)-3-(4-trifluoromethyl-phenyl)-acrylamide | | |
| 220 | (S)-N-(1-{[2-(Benzyl-methyl-amino)-ethyl]-methyl- | 0.89 | 691.79 |
| | carbamoyl}-2-phenyl-ethyl)-N-(4-pyridin-2-yl-benzyl)-3-(4- | | |
| | trifluoromethyl-phenyl)-acrylamide | | |
| 221 | (S)-N-[1-({2-[(6-Chloro-pyridin-3-ylmethyl)-methyl-amino]- | 0.87 | 726.73 |
| | ethyl}-methyl-carbamoyl)-2-phenyl-ethyl]-N-(4-pyridin-2-yl- | | |
| | benzyl)-3-(4-trifluoromethyl-phenyl)-acrylamide | | |
| 222 | (S)-N-(1-{[2-(Furan-3-ylmethyl-methyl-amino)-ethyl]-methyl- | 0.87 | 681.77 |
| | carbamoyl}-2-phenyl-ethyl)-N-(4-pyridin-2-yl-benzyl)-3-(4- | | |
| | trifluoromethyl-phenyl)-acrylamide | | |
| 223 | (S)-N-(1-{[2-(Furan-2-ylmethyl-methyl-amino)-ethyl]-methyl- | 0.87 | 681.76 |
| | carbamoyl}-2-phenyl-ethyl)-N-(4-pyridin-2-yl-benzyl)-3-(4- | | |
| | trifluoromethyl-phenyl)-acrylamide | | |
| 224 | (S)-N-(1-{Methyl-[2-(methyl-pyridin-2-ylmethyl-amino)-ethyl]- | 0.87 | 692.79 |
| | carbamoyl}-2-phenyl-ethyl)-N-(4-pyridin-2-yl-benzyl)-3-(4- | | |
| | trifluoromethyl-phenyl)-acrylamide | | |
| 225 | (S)-N-(1-{Methyl-[2-(methyl-thiophen-2-ylmethyl-amino)- | 0.88 | 697.74 |
| | ethyl]-carbamoyl}-2-phenyl-ethyl)-N-(4-pyridin-2-yl-benzyl)- | | |
| | 3-(4-trifluoromethyl-phenyl)-acrylamide | | |
| 226 | (S)-N-[1-({2-[(5-Chloro-thiophen-2-ylmethyl)-methyl-amino]- | 0.90 | 731.70 |
| | ethyl}-methyl-carbamoyl)-2-phenyl-ethyl]-N-(4-pyridin-2-yl- | | |
| | benzyl)-3-(4-trifluoromethyl-phenyl)-acrylamide | | |
| 227 | (S)-N-[1-({2-[(6-Bromo-pyridin-3-ylmethyl)-methyl-amino]- | 0.87 | 772.70 |
| | ethyl}-methyl-carbamoyl)-2-phenyl-ethyl]-N-(4-pyridin-2-yl- | | |
| | benzyl)-3-(4-trifluoromethyl-phenyl)-acrylamide | | |
| 228 | (S)-N-[1-({2-[(5-Hydroxymethyl-furan-2-ylmethyl)-methyl- | 0.85 | 711.33 |
| | amino]-ethyl}-methyl-carbamoyl)-2-phenyl-ethyl]-N-(4- | | |
| | pyridin-2-yl-benzyl)-3-(4-trifluoromethyl-phenyl)-acrylamide | | |
| 229 | (S)-N-[1-({2-[(6-Methoxy-pyridin-3-ylmethyl)-methyl-amino]- | 0.87 | 722.71 |
| | ethyl}-methyl-carbamoyl)-2-phenyl-ethyl]-N-(4-pyridin-2-yl- | | |
| | benzyl)-3-(4-trifluoromethyl-phenyl)-acrylamide | | |
| 230 | (S)-N-[1-(Methyl-{2-[methyl-(6-trifluoromethyl-pyridin-3- | 0.89 | 760.71 |
| | ylmethyl)-amino]-ethyl}-carbamoyl)-2-phenyl-ethyl]-N-(4- | | |
| | pyridin-2-yl-benzyl)-3-(4-trifluoromethyl-phenyl)-acrylamide | | |
| 231 | (S)-N-(1-{Methyl-[2-(methyl-pyridin-3-ylmethyl-amino)-ethyl]- | 0.81 | 692.73 |
| | carbamoyl}-2-phenyl-ethyl)-N-(4-pyridin-2-yl-benzyl)-3-(4- | | |
| | trifluoromethyl-phenyl)-acrylamide | | |
| 232 | (S)-N-(1-{Methyl-[2-(methyl-thiophen-3-ylmethyl-amino)- | 0.88 | 697.63 |
| | ethyl]-carbamoyl}-2-phenyl-ethyl)-N-(4-pyridin-2-yl-benzyl)- | | |
| | 3-(4-trifluoromethyl-phenyl)-acrylamide | | |
| 233 | (S)-N-[1-(Methyl-{2-[methyl-(2-methyl-benzyl)-amino]-ethyl}- | 0.91 | 705.68 |
| | carbamoyl)-2-phenyl-ethyl]-N-(4-pyridin-2-yl-benzyl)-3-(4- | | |
| | trifluoromethyl-phenyl)-acrylamide | | |
| 234 | (S)-N-[1-({2-[(2,4-Dimethyl-benzyl)-methyl-amino]-ethyl}- | 0.93 | 719.69 |
| | methyl-carbamoyl)-2-phenyl-ethyl]-N-(4-pyridin-2-yl-benzyl)- | | |
| | 3-(4-trifluoromethyl-phenyl)-acrylamide | | |
| 235 | (S)-N-[1-({2-[(3,5-Dimethoxy-benzyl)-methyl-amino]-ethyl}- | 1.02 | 759.37 |
| | methyl-carbamoyl)-2-phenyl-ethyl]-N-(4-morpholin-4-yl- | | |
| | benzyl)-3-(4-trifluoromethyl-phenyl)-acrylamide | | |
| 236 | (S)-N-[4-(4-Acetyl-piperazin-1-yl)-benzyl]-N-[1-({2-[(3,5- | 1.01 | 799.34 |
| | dimethoxy-benzyl)-methyl-amino]-ethyl}-methyl-carbamoyl)- | | |
| | 2-phenyl-ethyl]-3-(4-trifluoromethyl-phenyl)-acrylamide | | |
| 237 | (S)-N-[4-(4-Acetyl-piperazin-1-yl)-benzyl]-N-(1-{[2-({4-[(2- | 0.95 | 813.34 |
| | hydroxy-ethyl)-methyl-amino]-benzyl}-methyl-amino)-ethyl]- | | |
| | methyl-carbamoyl}-2-phenyl-ethyl)-3-(4-trifluoromethyl- | | |
| | phenyl)-acrylamide | | |
| 238 | (S)-N-[4-(4-Acetyl-piperazin-1-yl)-benzyl]-N-[1-({2-[(4- | 0.96 | 756.30 |
| | hydroxy-benzyl)-methyl-amino]-ethyl}-methyl-carbamoyl)-2- | | |
| | phenyl-ethyl]-3-(4-trifluoromethyl-phenyl)-acrylamide | | |
| 239 | (S)-N-[4-(4-Acetyl-piperazin-1-yl)-benzyl]-N-[1-({2-[(4- | 0.95 | 811.35 |
| | diethylamino-benzyl)-methyl-amino]-ethyl}-methyl- | | |
| | carbamoyl)-2-phenyl-ethyl]-3-(4-trifluoromethyl-phenyl)- | | |
| | acrylamide | | |
| 240 | (S)-N-[4-(4-Acetyl-piperazin-1-yl)-benzyl]-N-[1-({2-[(3- | 0.96 | 756.31 |
| | hydroxy-benzyl)-methyl-amino]-ethyl}-methyl-carbamoyl)-2- | | |
| | phenyl-ethyl]-3-(4-trifluoromethyl-phenyl)-acrylamide | | |
| 241 | (S)-N-[4-(4-Acetyl-piperazin-1-yl)-benzyl]-N-(1-{methyl-[2- | 0.92 | 741.37 |
| | (methyl-pyridin-3-ylmethyl-amino)-ethyl]-carbamoyl}-2- | | |
| | phenyl-ethyl)-3-(4-trifluoromethyl-phenyl)-acrylamide | | |
| 242 | (S)-N-[4-(4-Acetyl-piperazin-1-yl)-benzyl]-N-{1-[(2-{[3-(2- | 0.95 | 799.46 |
| | hydroxy-ethoxy)-benzyl]-methyl-amino}-ethyl)-methyl- | | |
| | carbamoyl]-2-phenyl-ethyl}-3-(4-trifluoromethyl-phenyl)- | | |
| | acrylamide | | |
| 243 | (S)-N-[4-(4-Acetyl-piperazin-1-yl)-benzyl]-N-[1-({2-[(3-cyano- | 0.99 | 765.27 |
| | benzyl)-methyl-amino]-ethyl}-methyl-carbamoyl)-2-phenyl- | | |
| | ethyl]-3-(4-trifluoromethyl-phenyl)-acrylamide | | |
| 244 | (S)-N-[4-(4-Acetyl-piperazin-1-yl)-benzyl]-N-[1-({2-[(4- | 1.03 | 798.25 |
| | isopropoxy-benzyl)-methyl-amino]-ethyl}-methyl-carbamoyl)- | | |
| | 2-phenyl-ethyl]-3-(4-trifluoromethyl-phenyl)-acrylamide | | |
| 245 | (S)-N-[4-(4-Acetyl-piperazin-1-yl)-benzyl]-N-[1-(methyl-{2- | 1.01 | 811.31 |
| | [methyl-(4-methyl-3,4-dihydro-2H-benzo[1,4]oxazin-7- | | |
| | ylmethyl)-amino]-ethyl}-carbamoyl)-2-phenyl-ethyl]-3-(4- | | |
| | trifluoromethyl-phenyl)-acrylamide | | |
| 246 | (S)-N-[4-(4-Acetyl-piperazin-1-yl)-benzyl]-N-{1-[(2-{[4-(3- | 0.87 | 841.37 |
| | dimethylamino-propoxy)-benzyl]-methyl-amino}-ethyl)- | | |
| | methyl-carbamoyl]-2-phenyl-ethyl}-3-(4-trifluoromethyl- | | |
| | phenyl)-acrylamide | | |
| 247 | (S)-N-[4-(4-Acetyl-piperazin-1-yl)-benzyl]-N-[1-({2-[(6- | 0.97 | 771.15 |
| | methoxy-pyridin-3-ylmethyl)-methyl-amino]-ethyl}-methyl- | | |
| | carbamoyl)-2-phenyl-ethyl]-3-(4-trifluoromethyl-phenyl)- | | |
| | acrylamide | | |
| 248 | (S)-N-[4-(4-Acetyl-piperazin-1-yl)-benzyl]-N-(1-{methyl-[2- | 0.95 | 747.27 |
| | (methyl-thiazol-2-ylmethyl-amino)-ethyl]-carbamoyl}-2- | | |
| | phenyl-ethyl)-3-(4-trifluoromethyl-phenyl)-acrylamide | | |
| 249 | (S)-N-[4-(4-Acetyl-piperazin-1-yl)-benzyl]-N-(1-{[2- | 1.00 | 784.23 |
| | (benzo[1,3]dioxol-5-ylmethyl-methyl-amino)-ethyl]-methyl- | | |
| | carbamoyl}-2-phenyl-ethyl)-3-(4-trifluoromethyl-phenyl)- | | |
| | acrylamide | | |
| 250 | (S)-N-[4-(4-Acetyl-piperazin-1-yl)-benzyl]-N-(1-{methyl-[2- | 0.88 | 742.40 |
| | (methyl-pyrimidin-5-ylmethyl-amino)-ethyl]-carbamoyl}-2- | | |
| | phenyl-ethyl)-3-(4-trifluoromethyl-phenyl)-acrylamide | | |
| 251 | (S)-N-[4-(4-Acetyl-piperazin-1-yl)-benzyl]-N-[1-({2-[(2,3- | 0.99 | 790.40 |
| | difluoro-4-methyl-benzyl)-methyl-amino]-ethyl}-methyl- | | |
| | carbamoyl)-2-phenyl-ethyl]-3-(4-trifluoromethyl-phenyl)- | | |
| | acrylamide | | |
| 252 | (S)-N-[4-(4-Acetyl-piperazin-1-yl)-benzyl]-N-[1-({2-[(3H- | 0.82 | 730.40 |
| | imidazol-4-ylmethyl)-methyl-amino]-ethyl}-methyl- | | |
| | carbamoyl)-2-phenyl-ethyl]-3-(4-trifluoromethyl-phenyl)- | | |
| | acrylamide | | |
| 253 | (S)-N-[4-(4-Acetyl-piperazin-1-yl)-benzyl]-N-(1-{methyl-[2- | 0.87 | 741.40 |
| | (methyl-pyridin-4-ylmethyl-amino)-ethyl]-carbamoyl}-2- | | |
| | phenyl-ethyl)-3-(4-trifluoromethyl-phenyl)-acrylamide | | |
| 254 | (S)-N-[4-(4-Acetyl-piperazin-1-yl)-benzyl]-N-(1-{[2-(benzyl- | 0.96 | 740.40 |
| | methyl-amino)-ethyl]-methyl-carbamoyl}-2-phenyl-ethyl)-3- | | |
| | (4-trifluoromethyl-phenyl)-acrylamide | | |
| 255 | (S)-N-(1-{[2-({4-[(2-Hydroxy-ethyl)-methyl-amino]-benzyl}- | 0.97 | 772.30 |
| | methyl-amino)-ethyl]-methyl-carbamoyl}-2-phenyl-ethyl)-N- | | |
| | (4-morpholin-4-yl-benzyl)-3-(4-trifluoromethyl-phenyl)- | | |
| | acrylamide | | |
| 256 | (S)-N-[1-({2-[(4-Hydroxy-benzyl)-methyl-amino]-ethyl}- | 0.97 | 715.30 |
| | methyl-carbamoyl)-2-phenyl-ethyl]-N-(4-morpholin-4-yl- | | |
| | benzyl)-3-(4-trifluoromethyl-phenyl)-acrylamide | | |
| 257 | (S)-N-[1-({2-[(4-Diethylamino-benzyl)-methyl-amino]-ethyl}- | 0.94 | 770.30 |
| | methyl-carbamoyl)-2-phenyl-ethyl]-N-(4-morpholin-4-yl- | | |
| | benzyl)-3-(4-trifluoromethyl-phenyl)-acrylamide | | |
| 258 | (S)-N-[1-({2-[(3-Hydroxy-benzyl)-methyl-amino]-ethyl}- | 0.97 | 715.30 |
| | methyl-carbamoyl)-2-phenyl-ethyl]-N-(4-morpholin-4-yl- | | |
| | benzyl)-3-(4-trifluoromethyl-phenyl)-acrylamide | | |
| 259 | (S)-N-(1-{Methyl-[2-(methyl-pyridin-3-ylmethyl-amino)-ethyl]- | 0.92 | 700.40 |
| | carbamoyl}-2-phenyl-ethyl)-N-(4-morpholin-4-yl-benzyl)-3- | | |
| | (4-trifluoromethyl-phenyl)-acrylamide | | |
| 260 | (S)-N-{1-[(2-{[3-(2-Hydroxy-ethoxy)-benzyl]-methyl-amino}- | 0.96 | 759.30 |
| | ethyl)-methyl-carbamoyl]-2-phenyl-ethyl}-N-(4-morpholin-4- | | |
| | yl-benzyl)-3-(4-trifluoromethyl-phenyl)-acrylamide | | |
| 261 | (S)-N-[1-({2-[(3-Cyano-benzyl)-methyl-amino]-ethyl}-methyl- | 1.00 | 724.30 |
| | carbamoyl)-2-phenyl-ethyl]-N-(4-morpholin-4-yl-benzyl)-3-(4- | | |
| | trifluoromethyl-phenyl)-acrylamide | | |
| 262 | (S)-N-[1-({2-[(4-Isopropoxy-benzyl)-methyl-amino]-ethyl}- | 1.05 | 757.30 |
| | methyl-carbamoyl)-2-phenyl-ethyl]-N-(4-morpholin-4-yl- | | |
| | benzyl)-3-(4-trifluoromethyl-phenyl)-acrylamide | | |
| 263 | (S)-N-[1-(Methyl-{2-[methyl-(4-methyl-3,4-dihydro-2H- | 1.02 | 770.30 |
| | benzo[1,4]oxazin-7-ylmethyl)-amino]-ethyl}-carbamoyl)-2- | | |
| | phenyl-ethyl]-N-(4-morpholin-4-yl-benzyl)-3-(4- | | |
| | trifluoromethyl-phenyl)-acrylamide | | |
| 264 | (S)-N-[1-({2-[(6-Methoxy-pyridin-3-ylmethyl)-methyl-amino]- | 0.98 | 730.30 |
| | ethyl}-methyl-carbamoyl)-2-phenyl-ethyl]-N-(4-morpholin-4- | | |
| | yl-benzyl)-3-(4-trifluoromethyl-phenyl)-acrylamide | | |
| 265 | (S)-N-(1-{Methyl-[2-(methyl-thiazol-2-ylmethyl-amino)-ethyl]- | 0.96 | 706.30 |
| | carbamoyl}-2-phenyl-ethyl)-N-(4-morpholin-4-yl-benzyl)-3- | | |
| | (4-trifluoromethyl-phenyl)-acrylamide | | |
| 266 | (S)-N-(1-{[2-(Benzo[1,3]dioxol-5-ylmethyl-methyl-amino)- | 1.01 | 743.20 |
| | ethyl]-methyl-carbamoyl}-2-phenyl-ethyl)-N-(4-morpholin-4- | | |
| | yl-benzyl)-3-(4-trifluoromethyl-phenyl)-acrylamide | | |
| 267 | (S)-N-(1-{Methyl-[2-(methyl-pyrimidin-5-ylmethyl-amino)- | 0.92 | 701.30 |
| | ethyl]-carbamoyl}-2-phenyl-ethyl)-N-(4-morpholin-4-yl- | | |
| | benzyl)-3-(4-trifluoromethyl-phenyl)-acrylamide | | |
| 268 | (S)-N-[1-({2-[(2,3-Difluoro-4-methyl-benzyl)-methyl-amino]- | 1.03 | 749.30 |
| | ethyl}-methyl-carbamoyl)-2-phenyl-ethyl]-N-(4-morpholin-4- | | |
| | yl-benzyl)-3-(4-trifluoromethyl-phenyl)-acrylamide | | |
| 269 | (S)-N-[1-({2-[(3H-Imidazol-4-ylmethyl)-methyl-amino]-ethyl}- | 0.85 | 689.30 |
| | methyl-carbamoyl)-2-phenyl-ethyl]-N-(4-morpholin-4-yl- | | |
| | benzyl)-3-(4-trifluoromethyl-phenyl)-acrylamide | | |
| 270 | (S)-N-(1-{Methyl-[2-(methyl-pyridin-4-ylmethyl-amino)-ethyl]- | 0.92 | 700.40 |
| | carbamoyl}-2-phenyl-ethyl)-N-(4-morpholin-4-yl-benzyl)-3- | | |
| | (4-trifluoromethyl-phenyl)-acrylamide | | |

*Analytic A, Zorbax SB-AQ column, acidic conditions

### Preparation of Compounds of Formula I Via Method E:

### Preparation of (4-Bromo-benzyl)-methyl-amine

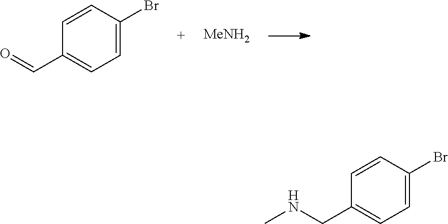

In an autoclave, a mixture of 4-bromobenzaldehyde (2.08 g, 11.15 mmol) and methylamine 2M solution in methanol (25 mL, 33.44 mmol) was stirred at 65° C. for 4 h. After cooling to rt, sodium borohydride (633 mg, 16.72 mmol) was added portionwise. The reaction mixture was stirred at rt for 30 min, then concentrated in vacuo. The resulting residue was dissolved in EA (30 mL) and the organic layer washed with a sat. NaHCO₃ solution (10 mL). The aq. phase was basified with few drops of 1N NaOH (pH=13) and extracted twice with EA. The combined organic extracts were washed with brine, dried (MgSO₄), filtered and concentrated under reduced pressure to afford the title compound as a colorless oil (2.04 g, 91%), which was used for the next step without further purification.

LC-MS (analytic A, Zorbax SB-AQ column, acidic conditions): t_{R}=0.61 min; [M+H]⁺=241.06 (MeCN adduct).

### Step 1

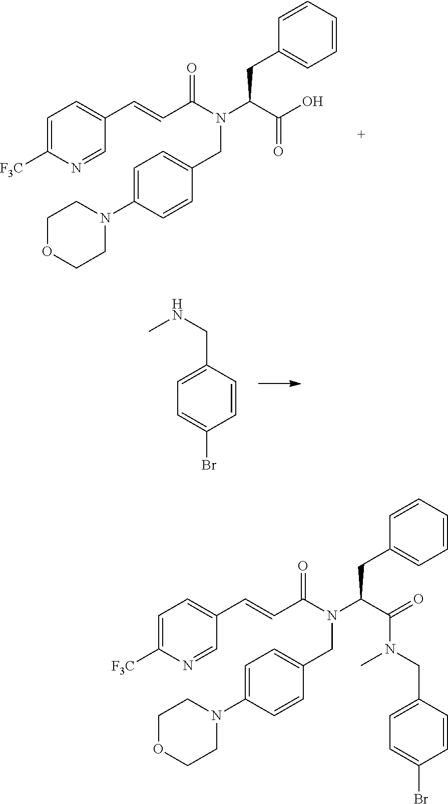

To a mixture of (S)-2-{(4-morpholin-4-yl-benzyl)-[3-(6-trifluoromethyl-pyridin-3-yl)-acryloyl]-amino}-3-phenyl-propionic acid (4.00 g, 7.41 mmol), TBTU (4.76 g, 14.83 mmol), and cat. DMAP in dry DCM (45 mL) was added DIPEA (3.8 mL, 22.24 mmol). The resulting mixture was stirred at rt for 10 min and then (4-bromo-benzyl)-methyl-amine (1.48 g, 7.41 mmol) was added. The reaction mixture was stirred at rt overnight under nitrogen atmosphere, then concentrated in vacuo. The resulting residue was taken up in EA. The organic layer was washed with water (5×) and brine, dried (MgSO₄), filtered and concentrated under reduced pressure. FC (n-heptane/EA 5:5) afforded the N-{1-[(4-bromo-benzyl)-methyl-carbamoyl]-2-phenyl-ethyl}-N-(4-morpholin-4-yl-benzyl)-3-(6-trifluoromethyl-pyridin-3-yl)-acrylamide as a yellow foam (2.38 g, 44%).

LC-MS (analytic A, Zorbax SB-AQ column, acidic conditions): t_{R}=1.16 min; [M+H]⁺=722.76.

### Step 2

### General Procedure 1

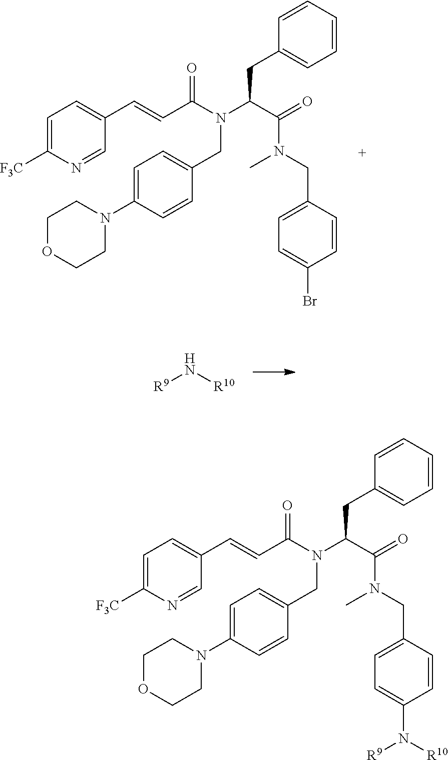

A mixture of N-{1-[(4-bromo-benzyl)-methyl-carbamoyl]-2-phenyl-ethyl}-N-(4-morpholin-4-yl-benzyl)-3-(6-trifluoromethyl-pyridin-3-yl)-acrylamide (1 eq), the amine NHR⁹R¹⁰ (1.5 eq), and sodium tert-butylate (1.5 eq) in dry dioxane (14 mL/mmol) was degassed with argon for 10 min and stirred at 105° C. Then a degassed solution (with argon) of the catalyst Solvias SK-CC02-A (0.06 eq in 125 mL/mmol dioxane) is added. The reaction mixture was stirred at 105° C. overnight then concentrated in vacuo. The resulting residue was taken up in EA, filtered over isolute and purified by preparative HPLC to afford the pure final compound.

### General Procedure 2

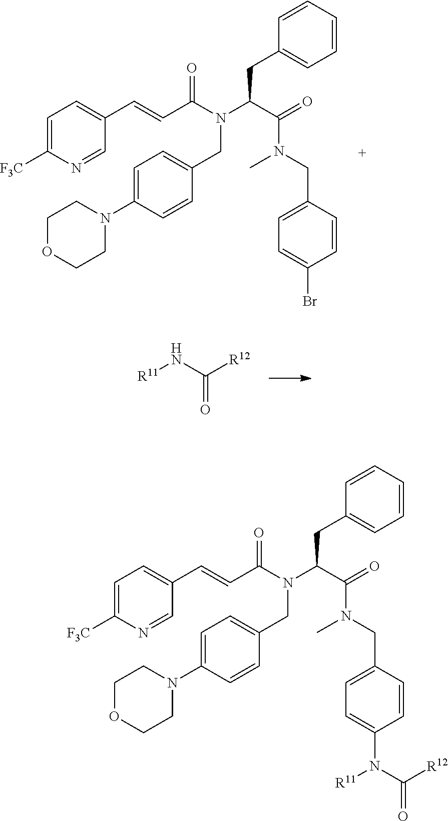

A mixture of N-{1-[(4-bromo-benzyl)-methyl-carbamoyl]-2-phenyl-ethyl}-N-(4-morpholin-4-yl-benzyl)-3-(6-trifluoromethyl-pyridin-3-yl)-acrylamide (1 eq), the amide NH(R¹¹)COR¹² (1.2 eq), potassium carbonate (2 eq), copper (I) iodide (0.05 eq), and N,N′-dimethylethylenediamine (0.1 eq) in dry dioxane (14 mL/mmol) was stirred at 120° C. overnight under nitrogen atmosphere. The reaction mixture was filtered over isolute using EA as solvent, then purified by preparative HPLC to afford the pure final compound.

| | | LC-MS* | |
|---|---|---|---|
| Ex. No. | Chemical name | t_{R} (min) | [M + H]⁺ |
| 271 | (S)-N-{1-[(4-Acetylamino-benzyl)-methyl-carbamoyl]-2- | 1.03 | 699.94 |
| | phenyl-ethyl}-N-(4-morpholin-4-yl-benzyl)-3-(6- | | |
| | trifluoromethyl-pyridin-3-yl)-acrylamide | | |
| 272 | (S)-N-(1-{Methyl-[4-(2-oxo-pyrrolidin-1-yl)-benzyl]- | 1.07 | 725.90 |
| | carbamoyl}-2-phenyl-ethyl)-N-(4-morpholin-4-yl- | | |
| | benzyl)-3-(6-trifluoromethyl-pyridin-3-yl)-acrylamide | | |
| 273 | (S)-Cyclopropanecarboxylic acid (4-{[methyl-(2-{(4- | 1.07 | 726.99 |
| | morpholin-4-yl-benzyl)-[3-(6-trifluoromethyl-pyridin-3- | | |
| | yl)-acryloyl]-amino}-3-phenyl-propionyl)-amino]- | | |
| | methyl}-phenyl)-amide | | |
| 274 | (S)-N-(1-{Methyl-[4-(2-oxo-piperidin-1-yl)-benzyl]- | 1.05 | 739.88 |
| | carbamoyl}-2-phenyl-ethyl)-N-(4-morpholin-4-yl- | | |
| | benzyl)-3-(6-trifluoromethyl-pyridin-3-yl)-acrylamide | | |
| 275 | (S)-N-(4-{[Methyl-(2-{(4-morpholin-4-yl-benzyl)-[3-(6- | 1.11 | 762.99 |
| | trifluoromethyl-pyridin-3-yl)-acryloyl]-amino}-3-phenyl- | | |
| | propionyl)-amino]-methyl}-phenyl)-benzamide | | |
| 276 | (S)-N-(1-{[4-(Acetyl-phenyl-amino)-benzyl]-methyl- | 1.10 | 776.00 |
| | carbamoyl}-2-phenyl-ethyl)-N-(4-morpholin-4-yl- | | |
| | benzyl)-3-(6-trifluoromethyl-pyridin-3-yl)-acrylamide | | |
| 277 | (S)-N-(1-{[4-(2-Methoxy-ethylamino)-benzyl]-methyl- | 1.00 | 716.00 |
| | carbamoyl}-2-phenyl-ethyl)-N-(4-morpholin-4-yl- | | |
| | benzyl)-3-(6-trifluoromethyl-pyridin-3-yl)-acrylamide | | |

*Analytic A, Zorbax SB-AQ column, acidic conditions

### Preparation of Compounds of Formula I Via Method F:

### Step 1

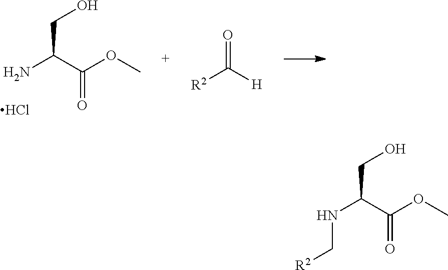

To a stirred suspension of L-serine methyl ester hydrochloride (1 eq) in dry DCM (1.5 mL/mmol) and TEA (1.1 eq) at rt were added successively anhydrous sodium sulfate (250 mg/mmol) and the aldehyde R²CHO (1 eq). The reaction mixture was stirred at rt for 20 h under nitrogen atmosphere, then filtered and concentrated in vacuo. The resulting solid was dissolved in dry MeOH (1.5 mL/mmol) and cooled to 0° C. before sodium borohydride (1.1 eq) was added. The reaction mixture was stirred at 0° C. for 2 h, then quenched with water and the MeOH was removed in vacuo. The resulting aq. solution was extracted with EA (3×) and the combined organic extracts were washed with brine, dried (MgSO₄), filtered and concentrated under reduced pressure to afford the secondary amine, which was used for the next step without further purification.

| | | | LC-MS* | |
|---|---|---|---|---|
| R² | Chemical name | Yield | t_{R} (min) | [M + H]⁺ |
| 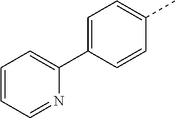 | (S)-3-Hydroxy-2-(4-pyridin-2-yl- benzylamino)-propionic acid methyl ester | 98% | 0.70 | 287.21 |
| 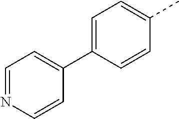 | (S)-3-Hydroxy-2-(4-pyridin-4-yl- benzylamino)-propionic acid methyl ester | quant. | 0.66 | 287.23 |

*Analytic A, Waters X-Bridge column, basic conditions

### Step 2

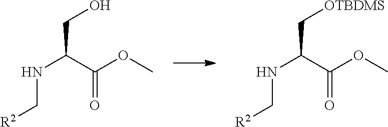

To a stirred solution of the serine derivative (1 eq) in dry DCM (4 mL/mmol) at 0° C. were added successively imidazole (1.5 eq) and TBDMSCl (1.1 eq). The reaction mixture was stirred at rt for 20 h under nitrogen atmosphere, then were added a sat. NH₄Cl solution and DCM. The aq. phase was separated and extracted twice with DCM. The combined organic extracts were dried (MgSO₄), filtered and concentrated under reduced pressure. FC (n-heptane/EA system) afforded the pure TBDMS-protected serine derivative.

| | | | LC-MS* | |
|---|---|---|---|---|
| R² | Chemical name | Yield | t_{R} (min) | [M + H]⁺ |
| 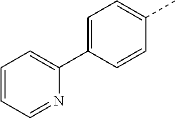 | (S)-3-(tert-Butyl-dimethyl-silanyloxy)-2- (4-pyridin-2-yl-benzylamino)-propionic acid methyl ester | 85% | 2.74 | 401.30 |
| 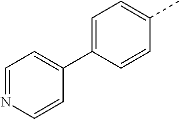 | (S)-3-(tert-Butyl-dimethyl-silanyloxy)-2- (4-pyridin-4-yl-benzylamino)-propionic acid methyl ester | 68% | 2.42 | 401.40 |

*Analytic B

### Step 3

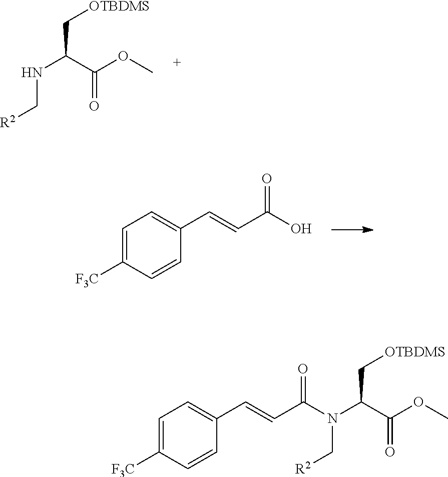

To a mixture of 4-(trifluoromethyl)cinnamic acid (1.05 eq) and DMF (few drops) in dry DCM (2.25 mL/mmol) was added dropwise oxalyl chloride (1.1 eq) at 0° C. The reaction mixture was stirred at 0° C. for 30 min then at rt for 4 h under nitrogen atmosphere. It was then cooled to 0° C. and treated with a solution of the amine (1 eq), TEA (2 eq), and DMAP (0.05 eq) in dry DCM (0.45 mL/mmol). The reaction mixture was stirred at rt for 17 h under nitrogen atmosphere, then water was added. The aq. phase was separated and extracted twice with DCM. The combined organic extracts were washed with a sat. NaHCO₃ solution, dried (MgSO₄), filtered and concentrated under reduced pressure. FC (n-heptane/EA system) afforded the pure amide.

| | | | LC-MS* | |
|---|---|---|---|---|
| R² | Chemical name | Yield | t_{R} (min) | [M + H]⁺ |
| 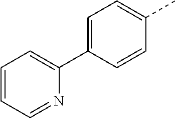 | (S)-3-(tert-Butyl-dimethyl-silanyloxy)-2- {(4-pyridin-2-yl-benzyl)-[3-(4- trifluoromethyl-phenyl)-acryloyl]-amino}- propionic acid methyl ester | 60% | 1.10 | 599.79 |
| 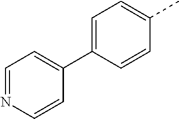 | (S)-3-(tert-Butyl-dimethyl-silanyloxy)-2- {(4-pyridin-4-yl-benzyl)-[3-(4- trifluoromethyl-phenyl)-acryloyl]-amino}- propionic acid methyl ester | 96% | 1.05 | 599.71 |

*Analytic A, Zorbax SB-AQ column, acidic conditions

### Step 4

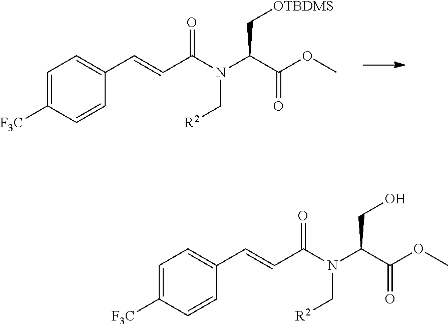

A mixture of the TBDMS-protected alcohol (1 eq) in AcOH/H₂O 2:1 (20 mL/mmol) was stirred at rt under nitrogen atmosphere for 1-3 days.

The solvent was removed in vacuo and the resulting residue dissolved in DCM and washed with a sat. NaHCO₃ solution. The aq. phase was extracted with DCM (3×). The combined organic extracts were dried (MgSO₄), filtered and concentrated under reduced pressure. Recrystallization in MeOH or EtOH afforded the pure alcohol.

| | | | LC-MS* | |
|---|---|---|---|---|
| R² | Chemical name | Yield | t_{R} (min) | [M + H]⁺ |
| 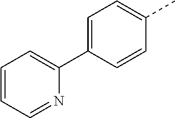 | (S)-3-Hydroxy-2-{(4-pyridin-2-yl-benzyl)- [3-(4-trifluoromethyl-phenyl)-acryloyl]- amino}-propionic acid methyl ester | 93% | 3.02 | 485.00 |
| 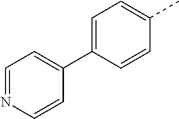 | (S)-3-Hydroxy-2-{(4-pyridin-4-yl-benzyl)- [3-(4-trifluoromethyl-phenyl)-acryloyl]- amino}-propionic acid methyl ester | 94% | 2.56 | 484.90 |

*Analytic B

### Step 5

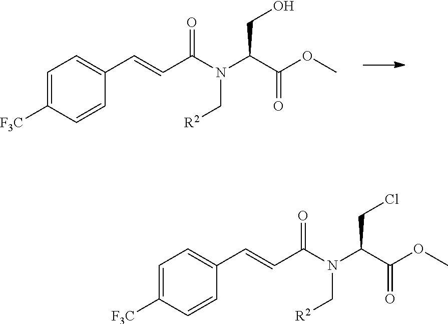

To a stirred suspension of the alcohol (1 eq) in dry DCM (14 mL/mmol) was added thionyl chloride (1.1 eq). The resulting yellow mixture was stirred at rt for 12 h under nitrogen atmosphere. The solution was washed with water and brine, dried (MgSO₄), filtered and concentrated under reduced pressure to afford the crude chloride derivative, which was used for the next step without further purification.

| | | | LC-MS* | |
|---|---|---|---|---|
| R² | Chemical name | Yield | t_{R} (min) | [M + H]⁺ |
| 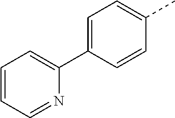 | (S)-3-Chloro-2-{(4-pyridin-2-yl-benzyl)- [3-(4-trifluoromethyl-phenyl)-acryloyl]- amino}-propionic acid methyl ester | 98% | 3.55 | 503.40 |
| 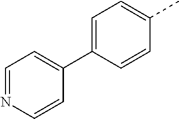 | (S)-3-Chloro-2-{(4-pyridin-4-yl-benzyl)- [3-(4-trifluoromethyl-phenyl)-acryloyl]- amino}-propionic acid methyl ester | quant. | 2.96 | 503.40 |

*Analytic B

### Step 6

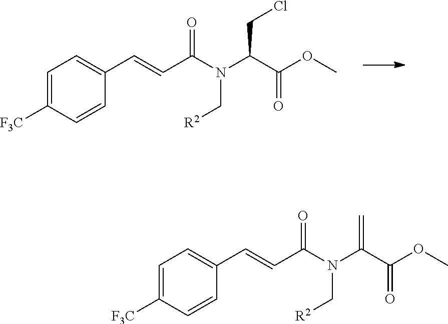

A mixture of the chloride (1 eq) and TEA (2 eq) in DCM (14 mL/mmol) was stirred at rt for 20 h under nitrogen atmosphere, then washed with water and brine, dried (MgSO₄), filtered and concentrated under reduced pressure to afford the crude elimination product, which was used for the next step without further purification.

| | | | LC-MS* | |
|---|---|---|---|---|
| R² | Chemical name | Yield | t_{R} (min) | [M + H]⁺ |
| 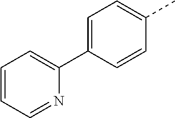 | 2-{(4-Pyridin-2-yl-benzyl)-[3-(4- trifluoromethyl-phenyl)-acryloyl]-amino}- acrylic acid methyl ester | crude | 3.41 | 467.10 |
| 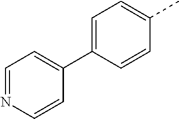 | 2-{(4-Pyridin-4-yl-benzyl)-[3-(4- trifluoromethyl-phenyl)-acryloyl]-amino}- acrylic acid methyl ester | crude | 2.89 | 467.20 |

*Analytic B

### Step 7

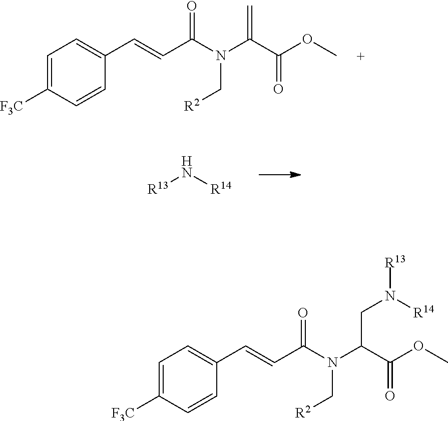

### General Procedure 1

A mixture of the acrylic acid methyl ester derivative (1 eq), the aliphatic cyclic amine NHR¹³R¹⁴ (2 eq), and FeCl₃ (0.1 eq) in dry DCM (5 mL/mmol) was stirred at rt for 60 h under nitrogen atmosphere. The reaction mixture was then washed with an aq. 1M Na₂SO₄ solution to eliminate iron species and the aq. phase extracted twice with DCM. The combined organic extracts were dried (MgSO₄), filtered and concentrated under reduced pressure. FC (n-heptane/EA or DCM/MeOH system) afforded the pure amino-acid derivative.

### General Procedure 2

To a stirred solution of the acrylic acid methyl ester derivative (1 eq) in dry MeCN (10 mL/mmol) was added potassium carbonate (6 eq) followed by the aromatic amine or carbamate or oxo-amide NHR¹³R¹⁴ (1.1 eq). The reaction mixture was stirred at rt for 4-15 h or was refluxed for 20-30 h under nitrogen atmosphere, then filtered and concentrated in under reduced pressure. FC (n-heptane/EA or DCM/MeOH system) afforded the pure amino-acid derivative.

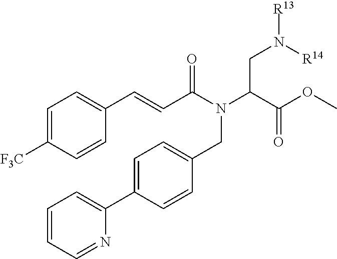

| 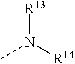 | Chemical name | Yield | LC-MS* t_{R} (min) [M + H]⁺ | |
|---|---|---|---|---|
| 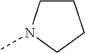 | rac-2-{(4-Pyridin-2-yl-benzyl)-[3-(4- trifluoromethyl-phenyl)-acryloyl]-amino}-3- pyrrolidin-1-yl-propionic acid methyl ester | quant. | 2.66 | 538.20 |
| 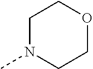 | rac-3-Piperidin-1-yl-2-{(4-pyridin-2-yl-benzyl)-[3- (4-trifluoromethyl-phenyl)-acryloyl]-amino}- propionic acid methyl ester | 83% | 2.70 | 554.30 |
| 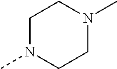 | rac-3-(4-Methyl-piperazin-1-yl)-2-{(4-pyridin-2- yl-benzyl)-[3-(4-trifluoromethyl-phenyl)-acryloyl]- amino}-propionic acid methyl ester | 72% | 2.58 | 567.50 |
| 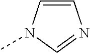 | rac-3-Imidazol-1-yl-2-{(4-pyridin-2-yl-benzyl)-[3- (4-trifluoromethyl-phenyl)-acryloyl]-amino}- propionic acid methyl ester | 84% | 2.57 | 535.40 |

*Analytic B

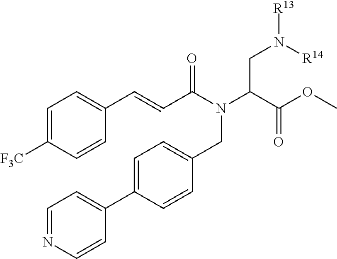

| 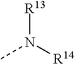 | Chemical name | Yield | LC-MS* t_{R} (min) [M + H]⁺ | |
|---|---|---|---|---|
| 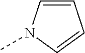 | rac-2-{(4-Pyridin-4-yl-benzyl)-[3-(4- trifluoromethyl-phenyl)-acryloyl)-amino}-3- pyrrol-1-yl-propionic acid methyl ester | 85% | 3.09 | 534.30 |
| 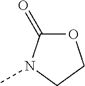 | rac-3-(2-Oxo-oxazolidin-3-yl)-2-{(4-pyridin-4-yl- benzyl)-[3-(4-trifluoromethyl-phenyl)-acryloyl]- amino}-propionic acid methyl ester | 76% | 2.65 | 554.20 |
| 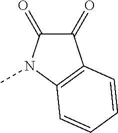 | rac-3-(2,3-Dioxo-2,3-dihydro-indol-1-yl)-2-{(4- pyridin-4-yl-benzyl)-[3-(4-trifluoromethyl- phenyl)-acryloyl]-amino}-propionic acid methyl ester | 84% | 2.92 | 614.10 |

*Analytic B

### Step 8

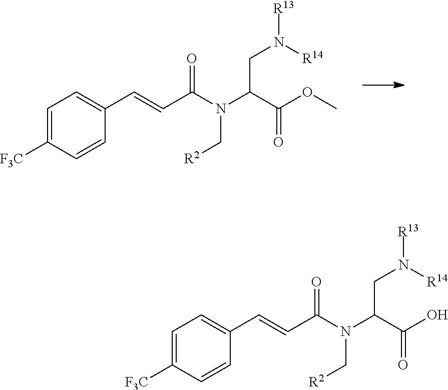

To a solution of the ester (1 eq) in MeOH (15 mL/mmol) was added dropwise aq. 2N NaOH (2-3.5 eq). The reaction mixture was stirred at rt for 2-4 h, then a few amount of water was added and the solvent was removed in vacuo. The residue was acidified with aq. 2N HCl until pH=2-3. The aq. phase was concentrated under reduced pressure to afford the crude acid, which was used for the next step without further purification.

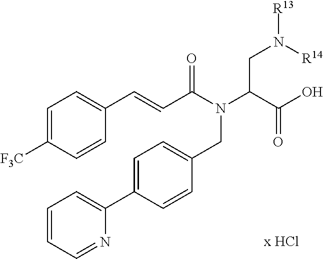

| 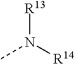 | Chemical name | Yield | LC-MS* t_{R} (min) [M + H]⁺ | |
|---|---|---|---|---|
| 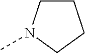 | rac-2-{(4-Pyridin-2-yl-benzyl)-[3-(4- trifluoromethyl-phenyl)-acryloyl]-amino}-3- pyrrolidin-1-yl-propionic acid, dihydrochloride salt | crude | 2.62 | 524.10 |
| 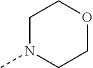 | rac-3-Piperidin-1-yl-2-{(4-pyridin-2-yl-benzyl)-[3- (4-trifluoromethyl-phenyl)-acryloyl]-amino}- propionic acid, dihydrochloride salt | 71% | 2.64 | 540.20 |
| 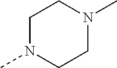 | rac-3-(4-Methyl-piperazin-1-yl)-2-{(4-pyridin-2- yl-benzyl)-[3-(4-trifluoromethyl-phenyl)-acryloyl]- amino}-propionic acid, trihydrochloride salt | 58% | 2.49 | 553.30 |
| 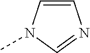 | rac-3-Imidazol-1-yl-2-{(4-pyridin-2-yl-benzyl)-[3- (4-trifluoromethyl-phenyl)-acryloyl)-amino}- propionic acid, dihydrochloride salt | quant. | 2.50 | 521.30 |

*Analytic B

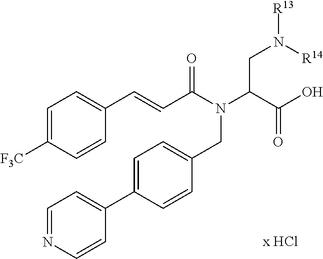

| 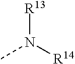 | Chemical name | Yield | LC-MS* t_{R} (min) [M + H]⁺ | |
|---|---|---|---|---|
| 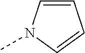 | rac-2-{(4-Pyridin-4-yl-benzyl)-[3-(4- trifluoromethyl-phenyl)-acryloyl]-amino}-3- pyrrol-1-yl-propionic acid, hydrochloride salt | quant. | 2.89 | 520.50 |
| 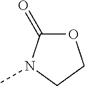 | rac-3-(2-Oxo-oxazolidin-3-yl)-2-{(4-pyridin-4-yl- benzyl)-[3-(4-trifluoromethyl-phenyl)-acryloyl]- amino}-propionic acid, hydrochloride salt | 91% | 2.49 | 540.00 |
| 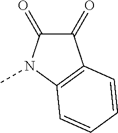 | rac-3-(2,3-Dioxo-2,3-dihydro-indol-1-yl)-2-{(4- pyridin-4-yl-benzyl)-[3-(4-trifluoromethyl- phenyl)-acryloyl]-amino}-propionic acid, hydrochloride salt | crude | 2.69 | 600.30 |

*Analytic B

### Step 9

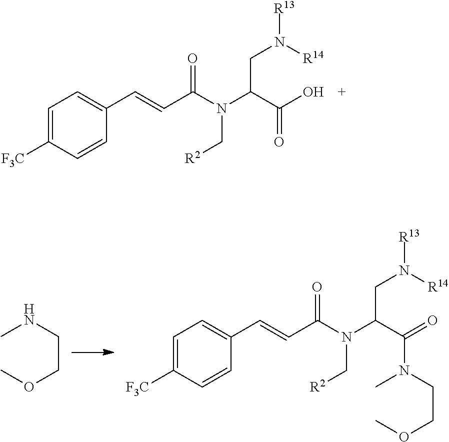

To a mixture of the acid (1 eq) and DIPEA (5 eq) in dry DCM (20 mL/mmol) was added TBTU (1.1 eq). After stirring at rt for 30 min under nitrogen atmosphere, N-(2-methoxyethyl)methylamine (1 eq) was added. The reaction mixture was stirred at rt under nitrogen atmosphere for 15-72 h, then water was added and the aq. phase extracted with DCM (2-5×). The combined organic extracts were washed with a sat. NaHCO₃ solution, dried (MgSO₄), filtered and concentrated under reduced pressure. The crude product was purified by preparative HPLC to afford the pure final compound.

Note: In the case of example 281, the imidazole elimination occurred. Thus, the aza-Michael addition of imidazole was repeated on the crude product according to the procedure 2 of step 7.

| | | LC-MS* | |
|---|---|---|---|
| Ex. No. | Chemical name | t_{R} (min) | [M + H]⁺ |
| 278 | rac-N-[1-[(2-Methoxy-ethyl)-methyl-carbamoyl]-2-(4- | 0.90 | 624.24 |
| | methyl-piperazin-1-yl)-ethyl]-N-(4-pyridin-2-yl-benzyl)-3- | | |
| | (4-trifluoromethyl-phenyl)-acrylamide | | |
| 279 | rac-N-{1-[(2-Methoxy-ethyl)-methyl-carbamoyl]-2- | 0.93 | 611.25 |
| | morpholin-4-yl-ethyl}-N-(4-pyridin-2-yl-benzyl)-3-(4- | | |
| | trifluoromethyl-phenyl)-acrylamide | | |
| 280 | rac-N-{1-[(2-Methoxy-ethyl)-methyl-carbamoyl]-2- | 0.99 | 595.10 |
| | pyrrolidin-1-yl-ethyl}-N-(4-pyridin-2-yl-benzyl)-3-(4- | | |
| | trifluoromethyl-phenyl)-acrylamide | | |
| 281 | rac-N-{2-Imidazol-1-yl-1-[(2-methoxy-ethyl)-methyl- | 0.89 | 592.12 |
| | carbamoyl]-ethyl}-N-(4-pyridin-2-yl-benzyl)-3-(4- | | |
| | trifluoromethyl-phenyl)-acrylamide | | |
| 282 | rac-N-{1-[(2-Methoxy-ethyl)-methyl-carbamoyl]-2-pyrrol- | 0.96 | 591.14 |
| | 1-yl-ethyl}-N-(4-pyridin-4-yl-benzyl)-3-(4-trifluoromethyl- | | |
| | phenyl)-acrylamide | | |
| 283 | rac-N-{2-(2,3-Dioxo-2,3-dihydro-indol-1-yl)-1-[(2- | 0.93 | 671.08 |
| | methoxy-ethyl)-methyl-carbamoyl]-ethyl}-N-(4-pyridin-4- | | |
| | yl-benzyl)-3-(4-trifluoromethyl-phenyl)acrylamide | | |
| 284 | rac-N-[1-[(2-Methoxy-ethyl)-methyl-carbamoyl]-2-(2-oxo- | 0.86 | 611.12 |
| | oxazolidin-3-yl)-ethyl]-N-(4-pyridin-4-yl-benzyl)-3-(4- | | |
| | trifluoromethyl-phenyl)-acrylamide | | |

*Analytic A, Waters X-Bridge column, basic conditions

In Vitro Antimalarial Activity: Plasmodium falciparum In Vitro Assay

In vitro activity against erythrocytic stages of P. falciparum is determined using a [³H] hypoxanthine incorporation assay. One strain resistant to chloroquine and pyrimethamine (P. falciparum K1) is used in the assays, and all test compounds are compared for activity with the standard drugs chloroquine (sigma C6628) and artemisinin (sigma-36, 159-3). Compounds are diluted in DMSO to 1 mM and added to parasite cultures incubated in RPMI 1640 medium without hypoxanthine, supplemented with HEPES (5.94 g/L), NaHCO₃ (2.1 g/L), neomycin (100 U/mL), Albumax (5 g/L) and washed human red cells at 2.5% haematocrit (0.3% parasitaemia). Seven serial doubling dilutions of each compound are prepared in 96-well microtitre plates and incubated in a humidifying atmosphere at 37° C.; 4% CO₂, 3% O₂, 93% N₂.

After 48 h, 50 μl of [3H] hypoxanthine (0.5 μCi) is added to each well of a plate. The plates are incubated for a further 24 h under the same conditions. The plates are then harvested with a Betaplate cell harvester (Wallac) and washed with distilled water. The dried filters are inserted into a plastic foil with 10 mL of scintillation fluid, and counted in a Betaplate liquid scintillation counter. IC₅₀ values are calculated from sigmoidal inhibition curves using Microsoft Excel. Inhibition activities (IC₅₀ values) of the 284 exemplified compounds are in the range of 1-494 nM with an average of 110 nM with respect to the Plasmodium falciparum strain K1.

TABLE 1
IC₅₀ values (nM) for the compounds of Examples 1-284:

| Compound of | |
|---|---|
| Example No.: | IC₅₀ (nM) on K1 |
| 1 | 211 |
| 2 | 466 |
| 3 | 436 |
| 4 | 323 |
| 5 | 319 |
| 6 | 54 |
| 7 | 14 |
| 8 | 381 |
| 9 | 69 |
| 10 | 176 |
| 11 | 315 |
| 12 | 220 |
| 13 | 355 |
| 14 | 226 |
| 15 | 163 |
| 16 | 115 |
| 17 | 113 |
| 18 | 273 |
| 19 | 250 |
| 20 | 116 |
| 21 | 14 |
| 22 | 60 |
| 23 | 141 |
| 24 | 103 |
| 25 | 90 |
| 26 | 465 |
| 27 | 67 |
| 28 | 81 |
| 29 | 34 |
| 30 | 34 |
| 31 | 54 |
| 32 | 298 |
| 33 | 319 |
| 34 | 433 |
| 35 | 317 |
| 36 | 173 |
| 37 | 279 |
| 38 | 417 |
| 39 | 170 |
| 40 | 490 |
| 41 | 169 |
| 42 | 104 |
| 43 | 354 |
| 44 | 14 |
| 45 | 4 |
| 46 | 1 |
| 47 | 1 |
| 48 | 8 |
| 49 | 43 |
| 50 | 13 |
| 51 | 90 |
| 52 | 92 |
| 53 | 14 |
| 54 | 17 |
| 55 | 65 |
| 56 | 58 |
| 57 | 89 |
| 58 | 50 |
| 59 | 121 |
| 60 | 48 |
| 61 | 464 |
| 62 | 138 |
| 63 | 87 |
| 64 | 180 |
| 65 | 269 |
| 66 | 494 |
| 67 | <8 |
| 68 | 156 |
| 69 | 169 |
| 70 | 144 |
| 71 | 54 |
| 72 | 344 |
| 73 | 156 |
| 74 | 40 |
| 75 | 11 |
| 76 | 163 |
| 77 | 23 |
| 78 | 47 |
| 79 | 288 |
| 80 | 44 |
| 81 | 102 |
| 82 | 99 |
| 83 | 209 |
| 84 | 27 |
| 85 | 31 |
| 86 | 27 |
| 87 | <8 |
| 88 | 45 |
| 89 | <8 |
| 90 | 43 |
| 91 | 26 |
| 92 | 23 |
| 93 | 13 |
| 94 | 15 |
| 95 | 33 |
| 96 | 20 |
| 97 | 15 |
| 98 | 98 |
| 99 | 17 |
| 100 | 12 |
| 101 | 8 |
| 102 | 6 |
| 103 | 438 |
| 104 | 45 |
| 105 | 44 |
| 106 | 470 |
| 107 | 70 |
| 108 | 225 |
| 109 | 100 |
| 110 | 324 |
| 111 | 214 |
| 112 | 153 |
| 113 | 84 |
| 114 | 107 |
| 115 | 32 |
| 116 | <8 |
| 117 | 117 |
| 118 | 104 |
| 119 | 81 |
| 120 | 285 |
| 121 | 28 |
| 122 | 156 |
| 123 | 296 |
| 124 | 110 |
| 125 | 439 |
| 126 | 160 |
| 127 | 21 |
| 128 | 94 |
| 129 | 19 |
| 130 | <8 |
| 131 | 282 |
| 132 | 28 |
| 133 | 82 |
| 134 | 33 |
| 135 | <8 |
| 136 | 74 |
| 137 | 413 |
| 138 | 340 |
| 139 | 155 |
| 140 | 191 |
| 141 | 339 |
| 142 | 460 |
| 143 | 5 |
| 144 | 24 |
| 145 | 5 |
| 146 | 9 |
| 147 | 77 |
| 148 | 65 |
| 149 | 26 |
| 150 | 319 |
| 151 | 373 |
| 152 | 242 |
| 153 | 268 |
| 154 | 420 |
| 155 | 327 |
| 156 | 382 |
| 157 | 10 |
| 158 | 24 |
| 159 | 49 |
| 160 | 93 |
| 161 | 49 |
| 162 | 45 |
| 163 | 37 |
| 164 | 247 |
| 165 | 76 |
| 166 | 96 |
| 167 | 41 |
| 168 | 349 |
| 169 | 312 |
| 170 | 192 |
| 171 | 80 |
| 172 | 89 |
| 173 | 52 |
| 174 | 430 |
| 175 | 88 |
| 176 | 51 |
| 177 | 84 |
| 178 | 10 |
| 179 | 9 |
| 180 | 38 |
| 181 | 10 |
| 182 | 18 |
| 183 | 60 |
| 184 | 13 |
| 185 | 19 |
| 186 | 58 |
| 187 | 7 |
| 188 | 53 |
| 189 | 9 |
| 190 | 7 |
| 191 | 11 |
| 192 | 19 |
| 193 | 20 |
| 194 | 89 |
| 195 | 81 |
| 196 | 16 |
| 197 | 29 |
| 198 | 65 |
| 199 | 34 |
| 200 | 11 |
| 201 | 32 |
| 202 | 14 |
| 203 | 12 |
| 204 | 9 |
| 205 | 16 |
| 206 | 81 |
| 207 | 40 |
| 208 | 57 |
| 209 | 31 |
| 210 | 28 |
| 211 | 18 |
| 212 | 20 |
| 213 | 72 |
| 214 | 23 |
| 215 | 86 |
| 216 | 94 |
| 217 | 32 |
| 218 | 40 |
| 219 | 52 |
| 220 | 29 |
| 221 | 20 |
| 222 | 13 |
| 223 | 25 |
| 224 | 29 |
| 225 | 20 |
| 226 | 75 |
| 227 | 18 |
| 228 | 9 |
| 229 | 9 |
| 230 | 10 |
| 231 | 30 |
| 232 | 33 |
| 233 | 53 |
| 234 | 64 |
| 235 | 58 |
| 236 | 68 |
| 237 | 55 |
| 238 | 27 |
| 239 | 61 |
| 240 | 28 |
| 241 | 27 |
| 242 | 14 |
| 243 | 52 |
| 244 | 19 |
| 245 | 87 |
| 246 | 64 |
| 247 | 26 |
| 248 | 21 |
| 249 | 39 |
| 250 | 27 |
| 251 | 53 |
| 252 | 80 |
| 253 | 19 |
| 254 | 76 |
| 255 | 59 |
| 256 | 39 |
| 257 | 65 |
| 258 | 43 |
| 259 | 32 |
| 260 | 14 |
| 261 | 48 |
| 262 | 17 |
| 263 | 72 |
| 264 | 39 |
| 265 | 36 |
| 266 | 58 |
| 267 | 40 |
| 268 | 80 |
| 269 | 63 |
| 270 | 25 |
| 271 | 100 |
| 272 | 80 |
| 273 | 75 |
| 274 | 63 |
| 275 | 59 |
| 276 | 65 |
| 277 | 47 |
| 278 | 137 |
| 279 | <8 |
| 280 | 219 |
| 281 | 448 |
| 282 | 35 |
| 283 | 324 |
| 284 | 362 |
| Chloroquine | 194 |
| Artemisinin | 3 |

### In Vivo Antimalarial Efficacy Studies

In vivo antimalarial activity is assessed for groups of three female NMRI mice (20-22 g) intravenously infected on day 0 with P. berghei strain GFP-ANKA (0.2 mL heparinized saline suspension containing 2×10⁷ parasitized erythrocytes). In control mice, parasitaemia typically rise to approximately 40% by day 3 after infection, and control mice die between day 5 and day 7 after infection. For the mice treated with compounds, the compounds are either formulated in an aqueous-gelatine vehicle with 3 mg/mL compounds or in tween 80/ethanol (7%/3%) with 5 mg/mL.

Compounds are administered intraperitonealy or subcoutaneously either as two consecutive twice-daily dosings (BID) (2×75 mg/kg BID, 24 and 48 hours after infection) or as four consecutive daily doses (4×10 mg/kg or 4×50 mg/kg, 3, 24, 48 and 72 hours after infection). With the double BID-dose regimen, 24 h after the last drug treatment, 1 μl tail blood is taken, resuspended in 1 mL PBS buffer and parasitemia determined with a FACScan (Becton Dickinson) by counting 100 000 red blood cells. Tail blood samples for the quadruple-dose regimen are processed on day 4 after infection. Activity is calculated as the difference between the mean value of the control and treated groups expressed as a percent relative to the control group. For parasetimias lower than 0.1%, the presence of parasites in the FACS gate is checked visually. The survival days of infected mice treated with compound is also recorded for each compound. Mice surviving for 30 days are checked for parasitemia and subsequently euthanised. A compound is considered curative if the animal survives to day 30 post-infection with no detectable parasites.

1. A compound of formula I: 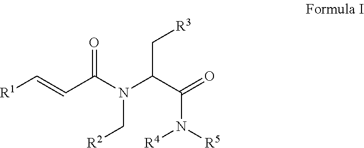
wherein
R¹ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, trifluoromethoxy, and amino, wherein the amino group is optionally mono- or di-substituted with (C₁-C₄)alkyl or mono-substituted with (C₁-C₄)alkyl-carbonyl; or R¹ represents aryl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl;
R² represents aryl or heteroaryl, wherein these two radicals can optionally be mono-, di-, tri-, or tetra-substituted, wherein the substituents are independently selected from the group consisting of halogen; (C₁-C₄)alkyl; (C₁-C₄)alkoxy; trifluoromethyl; trifluoromethoxy; heterocycloalkyl, that can optionally be mono-substituted on one nitrogen ring atom, if present, with (C₁-C₄)alkyl, or (C₁-C₄)alkyl-carbonyl; and aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
R³ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy; or R³ represents heterocycloalkyl that can optionally be mono-substituted on one nitrogen ring atom, if present, with (C₁-C₄)alkyl, cycloalkyl, (C₁-C₄)alkyl-carbonyl, or cycloalkyl-carbonyl; or R³ represents 2-oxo-oxazolidin-3-yl; or R³ represents 2,3-dioxo-2,3-dihydro-indol-1-yl that can optionally be mono-, di- or tri-substituted, wherein the substituents are independently selected from the group consisting of halogen, (C₁-C₄)alkyl, (C₁-C₄)alkoxy, trifluoromethyl, and trifluoromethoxy; and
R⁴ and R⁵, together with the nitrogen atom to which they are attached, form a morpholine ring; or together with the nitrogen atom to which they are attached, form the radicals 5,8-dihydro-6H-[1,7]naphthyridin-7-yl, 2,3-dihydro-1H-indol-1-yl, or 1,3-dihydro-1H-isoindol-2-yl, wherein these three radicals can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
or R⁴ and R⁵, together with the nitrogen atom to which they are attached, form a 3-amino-pyrrolidine ring, wherein the amino group is di-substituted with (C₁-C₄)alkyl; or together with the nitrogen atom to which they are attached, form a 3- or 4-substituted piperidine ring, wherein the substituent is selected from the group consisting of phenyl, benzyl, pyrrolidinomethyl, piperidinomethyl, amino di-substituted with (C₁-C₄)alkyl, and aminomethyl wherein the amino group is di-substituted with (C₁-C₄)alkyl;
or R⁴ represents hydrogen or (C₁-C₄)alkyl, and R⁵ represents 1-benzyl-pyrrolidin-3-yl or 1-aza-bicyclo[2.2.2]oct-3-yl;
or R⁴ represents (C₁-C₄)alkyl and R⁵ represents the following group: 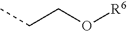
wherein R⁶ represents hydrogen, (C₁-C₄)alkyl, (C₃-C₄)alkenyl, cyanomethyl, carbamoylmethyl, cycloalkylmethyl, or 2-benzyloxy-ethyl; or R⁶ represents heteroaryl that can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; or R⁶ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cyano, trifluoromethyl, difluoromethoxy, and trifluoromethoxy;
or R⁴ represents hydrogen, (C₁-C₄)alkyl, or benzyl, and R⁵ represents the following group: 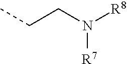
wherein R⁷ represents (C₁-C₄)alkyl; and R⁸ represents (C₁-C₄)alkyl or 4-methyl-3,4-dihydro-2H-benzo[1,4]oxazin-7-ylmethyl; or R⁸ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cycloalkyl, hydroxy, hydroxymethyl, cyano, trifluoromethyl, trifluoromethoxy, —O—(CH₂)₂—OH, —O—(CH₂)₃—N((C₁-C₄)alkyl)₂, and amino, wherein the amino group is mono- or di-substituted with substituents independently selected from (C₁-C₄)alkyl and hydroxy-(C₁-C₄)alkyl; or R⁸ represents arylmethyl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl; or R⁷ and R⁸, together with the nitrogen atom to which they are attached, form a piperidine, morpholine, or azepane ring;
or R⁴ represents (C₁-C₄)alkyl and R⁵ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
or R⁴ represents (C₁-C₄)alkyl and R⁵ represents the following group: 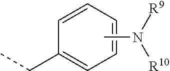
wherein the amino group can be in position 2, 3 or 4; R⁹ represents hydrogen, phenyl, or (C₁-C₄)alkyl; and R¹⁰ represents (C₁-C₄)alkyl, —(CH₂)₂—O—(C₁-C₄)alkyl, (C₁-C₄)alkyl-carbonyl, cycloalkyl-carbonyl, or benzoyl; or R⁹ and R¹⁰, together with the nitrogen atom to which they are attached, form a pyrrolidin-2-one or a piperidin-2-one ring, in a free or a pharmaceutically acceptable salt form.

2. The compound according to claim 1, wherein the carbon atom to which —CH₂—R³ is attached is in the (S)-configuration: 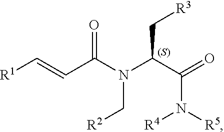
in a free or a pharmaceutically acceptable salt form.

3. The compound according to claim 1, wherein:
R¹ represents mono-substituted aryl or mono-substituted heteroaryl, wherein the substituent is selected from the group consisting of halogen, (C₁-C₄)alkyl, (C₁-C₄)alkoxy, cycloalkyl, trifluoromethyl, and trifluoromethoxy,
in a free or a pharmaceutically acceptable salt form.

4. The compound according to claim 3, wherein:
R¹ represents mono-substituted aryl or mono-substituted heteroaryl, wherein the substituent is selected from the group consisting of chlorine, methyl, methoxy, and trifluoromethyl,
in a free or a pharmaceutically acceptable salt form.

5. The compound according to claim 1, wherein:
R² represents mono-substituted aryl or mono-substituted heteroaryl, wherein the substituent is selected from the group consisting of halogen, (C₁-C₄)alkyl, (C₁-C₄)alkoxy, trifluoromethyl, trifluoromethoxy, aryl, heteroaryl, and heterocycloalkyl wherein the heterocycloalkyl can optionally be mono-substituted on one nitrogen ring atom, if present, with (C₁-C₄)alkyl or (C₁-C₄)alkyl-carbonyl,
in a free or a pharmaceutically acceptable salt form.

6. The compound according to claim 1, wherein:
R³ represents phenyl, morpholin-4-yl, pyrrol-1-yl, or 1-methyl-1H-pyrazol-3-yl,
in a free or a pharmaceutically acceptable salt form.

7. The compound according to claim 1, wherein:
R⁴ and R⁵, together with the nitrogen atom to which they are attached, form a 4-substituted piperidine ring, wherein the substituent is phenyl or benzyl,
in a free or a pharmaceutically acceptable salt form.

8. The compound according to claim 1, wherein:
R⁴ represents (C₁-C₄)alkyl and R⁵ represents the following group: 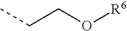
wherein R⁶ represents hydrogen, (C₁-C₄)alkyl, (C₃-C₄)alkenyl, cyanomethyl, carbamoylmethyl, cycloalkylmethyl, or 2-benzyloxy-ethyl; or R⁶ represents heteroaryl that can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; or R⁶ represents arylmethyl or heteroarylmethyl, wherein aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cyano, trifluoromethyl, difluoromethoxy, and trifluoromethoxy;
or R⁴ represents (C₁-C₄)alkyl and R⁵ represents the following group: 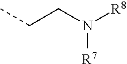
wherein R⁷ represents (C₁-C₄)alkyl; and R⁸ represents (C₁-C₄)alkyl or 4-methyl-3,4-dihydro-2H-benzo[1,4]oxazin-7-ylmethyl; or R⁸ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, cycloalkyl, hydroxy, hydroxymethyl, cyano, trifluoromethyl, trifluoromethoxy, —O—(CH₂)₂—OH, —O—(CH₂)₃—N((C₁-C₄)alkyl)₂, and amino, wherein the amino group is mono- or di-substituted with substituents independently selected from (C₁-C₄)alkyl and hydroxy-(C₁-C₄)alkyl; or R⁸ represents arylmethyl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl;
or R⁴ represents (C₁-C₄)alkyl and R⁵ represents arylmethyl or heteroarylmethyl, wherein the aryl or heteroaryl moiety can optionally be mono-, di-, tri-, or tetra-substituted, wherein the substituents are independently selected from the group consisting of halogen, (C₁-C₄)alkyl, (C₁-C₄)alkoxy, trifluoromethyl, and trifluoromethoxy;
or R⁴ represents (C₁-C₄)alkyl and R⁵ represents the following group: 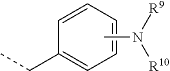
wherein the amino group can be in position 2, 3 or 4; R⁹ represents hydrogen, phenyl, or (C₁-C₄)alkyl; and R¹⁰ represents (C₁-C₄)alkyl, —(CH₂)₂—O—(C₁-C₄)alkyl, (C₁-C₄)alkyl-carbonyl, cycloalkyl-carbonyl, or benzoyl; or R⁹ and R¹⁰, together with the nitrogen atom to which they are attached, form a pyrrolidin-2-one or a piperidin-2-one ring,
in a free or a pharmaceutically acceptable salt form.

9. The compound according to claim 1 wherein:
R¹ represents phenyl, pyridyl, pyrimidyl, pyridazinyl, pyrazolyl, oxazolyl, thiazolyl, imidazolyl, isoxazolyl, or thiadiazolyl, wherein these radicals can optionally be mono-, di-, or tri-substituted, wherein the substituents are independently selected from the group consisting of halogen, (C₁-C₄)alkyl, (C₁-C₄)alkoxy, and trifluoromethyl;
R² represents phenyl or pyridyl, wherein these two radicals can optionally be mono-substituted, wherein the substituent is selected from the group consisting of (C₁-C₄)alkyl, morpholin-4-yl, 4-acetyl-piperazin-1-yl, pyridyl, and pyrimidyl;
R³ represents phenyl, pyrimidyl, imidazolyl, pyrrolyl, isoxazolyl, or pyrazolyl, wherein these radicals can optionally be mono-substituted with (C₁-C₄)alkyl; or R³ represents pyrrolidinyl, morpholinyl, or piperazinyl that can optionally be mono-substituted on one nitrogen ring atom with (C₁-C₄)alkyl; or R³ represents 2-oxo-oxazolidin-3-yl or 2,3-dioxo-2,3-dihydro-indol-1-yl; and
R⁴ and R⁵, together with the nitrogen atom to which they are attached, form a morpholine ring; or together with the nitrogen atom to which they are attached, form the radicals 5,8-dihydro-6H-[1,7]naphthyridin-7-yl, 2,3-dihydro-1H-indol-1-yl, or 1,3-dihydro-1H-isoindol-2-yl;
or R⁴ and R⁵, together with the nitrogen atom to which they are attached, form a 3-amino-pyrrolidine ring, wherein the amino group is di-substituted with (C₁-C₄)alkyl; or
together with the nitrogen atom to which they are attached, form a 4-substituted piperidine ring, wherein the substituent is selected from the group consisting of phenyl, benzyl, pyrrolidinomethyl, amino di-substituted with (C₁-C₄)alkyl, and aminomethyl wherein the amino group is di-substituted with (C₁-C₄)alkyl;
or R⁴ represents hydrogen or (C₁-C₄)alkyl, and R⁵ represents 1-benzyl-pyrrolidin-3-yl or 1-aza-bicyclo[2.2.2]oct-3-yl;
or R⁴ represents (C₁-C₄)alkyl and R⁵ represents the following group: 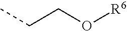
wherein R⁶ represents hydrogen, (C₁-C₄)alkyl, (C₃-C₄)alkenyl, cyanomethyl, carbamoylmethyl, cycloalkylmethyl, or 2-benzyloxy-ethyl; or R⁶ represents pyrimidyl; or
R⁶ represents benzyl, pyridylmethyl, furanylmethyl, isoxazolylmethyl, or benzotriazolylmethyl, wherein these radicals can optionally be mono- or di-substituted at the ring(s), wherein the substituents are independently selected from the group consisting of halogen, (C₁-C₄)alkyl, (C₁-C₄)alkoxy, cyano, trifluoromethyl, difluoromethoxy, and trifluoromethoxy;
or R⁴ represents hydrogen, (C₁-C₄)alkyl, or benzyl, and R⁵ represents the following group: 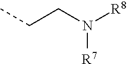
wherein R⁷ represents (C₁-C₄)alkyl; and R⁸ represents (C₁-C₄)alkyl or 4-methyl-3,4-dihydro-2H-benzo[1,4]oxazin-7-ylmethyl; or R⁸ represents benzyl, pyridylmethyl, pyrimidylmethyl, furanylmethyl, thienylmethyl, thiazolylmethyl, or imidazolylmethyl, wherein these radicals can optionally be mono-, di-, or tri-substituted at the ring, wherein the substituents are independently selected from the group consisting of halogen, (C₁-C₄)alkyl, (C₁-C₄)alkoxy, hydroxy, hydroxymethyl, cyano, trifluoromethyl, —O—(CH₂)₂—OH, —O—(CH₂)₃—N((C₁-C₄)alkyl)₂, and amino, wherein the amino group is di-substituted with substituents independently selected from (C₁-C₄)alkyl, and hydroxy-(C₁-C₄)alkyl; or
R⁸ represents phenylmethyl wherein two adjacent carbon ring atoms of the phenyl moiety are substituted with (C₁-C₂)alkylenedioxy; or R⁷ and R⁸, together with the nitrogen atom to which they are attached, form a piperidine, morpholine, or azepane ring;
or R⁴ represents (C₁-C₄)alkyl and R⁵ represents phenylmethyl, wherein the phenyl moiety is mono-substituted with (C₁-C₄)alkoxy;
or R⁴ represents (C₁-C₄)alkyl and R⁵ represents the following group: 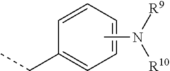
wherein the amino group is in position 4; R⁹ represents hydrogen or phenyl; and R¹⁰ represents —(CH₂)₂—O—(C₁-C₄)alkyl, (C₁-C₄)alkyl-carbonyl, cycloalkyl-carbonyl, or benzoyl; or R⁹ and R¹⁰, together with the nitrogen atom to which they are attached, form a pyrrolidin-2-one or a piperidin-2-one ring,
in a free or a pharmaceutically acceptable salt form.

10. The compound according to claim 1 wherein:
R¹ represents phenyl, pyridyl, pyrimidyl or pyridazinyl, wherein these four radicals are mono-substituted, wherein the substituent is selected from the group consisting of halogen, (C₁-C₄)alkyl, (C₁-C₄)alkoxy, and trifluoromethyl; or R¹ represents 1-methyl-1H-pyrazol-3-yl, 1,5-dimethyl-1H-pyrazol-4-yl, 2,5-dimethyl-2H-pyrazol-3-yl, 1,3,5-trimethyl-1H-pyrazol-4-yl, 2-methyl-thiazol-4-yl, 2,4-dimethyl-thiazol-5-yl, 5-methyl-isoxazol-3-yl, 3,5-dimethyl-isoxazol-4-yl, 2,5-dimethyl-oxazol-4-yl, 2,3-dimethyl-3H-imidazol-4-yl, or [1,2,3]thiadiazol-4-yl;
R² represents phenyl or pyridyl, wherein these two radicals can optionally be mono-substituted with (C₁-C₄)alkyl, pyridyl, pyrimidyl, morpholinyl, or piperazinyl which is mono-substituted on one nitrogen ring atom with (C₁-C₄)alkyl-carbonyl;
R³ represents phenyl, morpholinyl, pyrrolyl, or 1-methyl-1H-pyrazol-3-yl; and
R⁴ and R⁵, together with the nitrogen atom to which they are attached, form a morpholine ring; or together with the nitrogen atom to which they are attached, form the radicals 5,8-dihydro-6H-[1,7]naphthyridin-7-yl, 2,3-dihydro-1H-indol-1-yl, or 1,3-dihydro-1H-isoindol-2-yl;
or R⁴ and R⁵, together with the nitrogen atom to which they are attached, form a 3-amino-pyrrolidine ring, wherein the amino group is di-substituted with (C₁-C₄)alkyl; or together with the nitrogen atom to which they are attached, form a 3- or 4-substituted piperidine ring, wherein the substituent is independently selected from the group consisting of phenyl, benzyl, pyrrolidinomethyl, amino di-substituted with (C₁-C₄)alkyl, and aminomethyl wherein the amino group is di-substituted with (C₁-C₄)alkyl;
or R⁴ represents (C₁-C₄)alkyl and R⁵ represents 1-benzyl-pyrrolidin-3-yl;
or R⁴ represents (C₁-C₄)alkyl and R⁵ represents the following group: 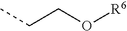
wherein R⁶ represents hydrogen, (C₁-C₄)alkyl, (C₃-C₄)alkenyl, cyanomethyl, carbamoylmethyl, cycloalkylmethyl, or 2-benzyloxy-ethyl; or R⁶ represents pyrimidyl; or
R⁶ represents phenylmethyl or pyridylmethyl, wherein the phenyl or pyridyl moiety can optionally be mono- or di-substituted, wherein the substituents are independently selected from the group consisting of halogen, (C₁-C₄)alkyl, (C₁-C₄)alkoxy, cyano, difluoromethoxy, and trifluoromethoxy; or R⁶ represents 5-trifluoromethyl-furan-3-ylmethyl, 5-methyl-isoxazol-3-ylmethyl, or 1-methyl-1H-benzotriazol-5-ylmethyl;
or R⁴ represents (C₁-C₄)alkyl and R⁵ represents the following group: 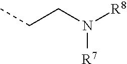
wherein R⁷ represents (C₁-C₄)alkyl; and R⁸ represents (C₁-C₄)alkyl; or R⁸ represents phenylmethyl or pyridylmethyl, wherein the phenyl or pyridyl moiety can optionally be mono-, di-, or tri-substituted, wherein the substituents are independently selected from the group consisting of halogen, (C₁-C₄)alkyl, (C₁-C₄)alkoxy, hydroxy, cyano, trifluoromethyl, —O—(CH₂)₂—OH, —O—(CH₂)₃—N((C₁-C₄)alkyl)₂, and amino, wherein the amino group is di-substituted wherein the substituents are independently selected from (C₁-C₄)alkyl and hydroxy-(C₁-C₄)alkyl; or R⁸ represents pyrimidylmethyl; or R⁸ represents furan-2-ylmethyl, furan-3-ylmethyl, 5-bromo-furan-2-ylmethyl, 5-hydroxymethyl-furan-2-ylmethyl, thiophen-2-ylmethyl, thiophen-3-ylmethyl, 5-chloro-thiophen-2-ylmethyl, thiazol-2-ylmethyl, 3H-imidazol-4-ylmethyl, or 4-methyl-3,4-dihydro-2H-benzo[1,4]oxazin-7-ylmethyl; or R⁸ represents phenylmethyl, wherein two adjacent carbon ring atoms of the phenyl moiety are substituted with (C₁-C₂)alkylenedioxy;
or R⁴ represents (C₁-C₄)alkyl and R⁵ represents phenylmethyl, wherein the phenyl moiety is mono-substituted with (C₁-C₄)alkoxy;
or R⁴ represents (C₁-C₄)alkyl and R⁵ represents the following group: 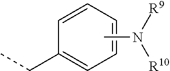
wherein the amino group can be in position 2, 3, or 4; R⁹ represents hydrogen or phenyl; and R¹⁰ represents —(CH₂)₂—O—(C₁-C₄)alkyl, (C₁-C₄)alkyl-carbonyl, cycloalkyl-carbonyl, or benzoyl; or R⁹ and R¹⁰, together with the nitrogen atom to which they are attached, form a pyrrolidin-2-one or a piperidin-2-one ring,
in a free or a pharmaceutically acceptable salt form.

11. The compound according to claim 1, selected from the group consisting of:
(S)—N-[1-Benzyl-2-(4-dimethylamino-piperidin-1-yl)-2-oxo-ethyl]-N-(4-pyridin-2-yl-benzyl)-3-(4-trifluoromethyl-phenyl)-acrylamide;
(S)—N-[1-Benzyl-2-(4-dimethylamino-piperidin-1-yl)-2-oxo-ethyl]-N-(4-pyridin-2-yl-benzyl)-3-(6-trifluoromethyl-pyridin-3-yl)-acrylamide;
(S)—N-[1-Benzyl-2-(4-dimethylamino-piperidin-1-yl)-2-oxo-ethyl]-N-(4-pyridin-2-yl-benzyl)-3-(1,3,5-trimethyl-1H-pyrazol-4-yl)-acrylamide;
(S)—N-[1-Benzyl-2-(4-dimethylamino-piperidin-1-yl)-2-oxo-ethyl]-N-(4-pyridin-4-yl-benzyl)-3-(1,3,5-trimethyl-1H-pyrazol-4-yl)-acrylamide;
(S)—N-[1-Benzyl-2-(4-dimethylamino-piperidin-1-yl)-2-oxo-ethyl]-3-(2,4-dimethyl-thiazol-5-yl)-N-(4-pyridin-4-yl-benzyl)-acrylamide;
(S)—N-[1-Benzyl-2-(4-dimethylamino-piperidin-1-yl)-2-oxo-ethyl]-3-(2,5-dimethyl-oxazol-4-yl)-N-(4-pyridin-4-yl-benzyl)-acrylamide;
(S)—N-[1-Benzyl-2-(4-dimethylamino-piperidin-1-yl)-2-oxo-ethyl]-3-(6-methyl-pyridin-3-yl)-N-(4-pyridin-4-yl-benzyl)-acrylamide;
(S)—N-[1-Benzyl-2-(4-dimethylamino-piperidin-1-yl)-2-oxo-ethyl]-3-(5-methyl-pyridin-2-yl)-N-(4-pyridin-4-yl-benzyl)-acrylamide;
(S)—N-[1-Benzyl-2-(4-dimethylamino-piperidin-1-yl)-2-oxo-ethyl]-3-(6-methoxy-pyridin-3-yl)-N-(4-pyridin-4-yl-benzyl)-acrylamide;
(S)—N-[1-Benzyl-2-(4-dimethylamino-piperidin-1-yl)-2-oxo-ethyl]-N-(4-pyridin-4-yl-benzyl)-3-(5-trifluoromethyl-pyridin-2-yl)-acrylamide;
(S)—N-[1-Benzyl-2-(4-dimethylamino-piperidin-1-yl)-2-oxo-ethyl]-3-(1,5-dimethyl-1H-pyrazol-4-yl)-N-(4-pyridin-4-yl-benzyl)-acrylamide;
(S)—N-[1-Benzyl-2-(4-benzyl-piperidin-1-yl)-2-oxo-ethyl]-N-pyridin-2-ylmethyl-3-(4-trifluoromethyl-phenyl)-acrylamide;
(S)—N-Benzyl-N-[1-benzyl-2-(4-benzyl-piperidin-1-yl)-2-oxo-ethyl]-3-(4-trifluoromethyl-phenyl)-acrylamide;
(S)—N-[1-Benzyl-2-(4-benzyl-piperidin-1-yl)-2-oxo-ethyl]-N-(4-pyridin-2-yl-benzyl)-3-(4-trifluoromethyl-phenyl)-acrylamide;
(S)—N-[1-Benzyl-2-(4-benzyl-piperidin-1-yl)-2-oxo-ethyl]-N-(4-pyridin-3-yl-benzyl)-3-(6-trifluoromethyl-pyridin-3-yl)-acrylamide;
(S)—N-Benzyl-N-[1-benzyl-2-(4-benzyl-piperidin-1-yl)-2-oxo-ethyl]-3-(6-trifluoromethyl-pyridin-3-yl)-acrylamide;
(S)—N-[1-Benzyl-2-oxo-2-(4-phenyl-piperidin-1-yl)-ethyl]-N-(4-pyrimidin-5-yl-benzyl)-3-(6-trifluoromethyl-pyridin-3-yl)-acrylamide;
(S)—N-[1-Benzyl-2-oxo-2-(4-phenyl-piperidin-1-yl)-ethyl]-N-(4-pyridin-2-yl-benzyl)-3-(6-trifluoromethyl-pyridin-3-yl)-acrylamide;
(S)—N-[1-Benzyl-2-(4-dimethylaminomethyl-piperidin-1-yl)-2-oxo-ethyl]-N-(4-pyridin-2-yl-benzyl)-3-(6-trifluoromethyl-pyridin-3-yl)-acrylamide;
(S)—N-[1-Benzyl-2-oxo-2-(4-pyrrolidin-1-ylmethyl-piperidin-1-yl)-ethyl]-N-(4-pyridin-2-yl-benzyl)-3-(4-trifluoromethyl-phenyl)-acrylamide;
N—[(S)-1-Benzyl-2-((R)-3-dimethylamino-pyrrolidin-1-yl)-2-oxo-ethyl]-N-(4-pyridin-2-yl-benzyl)-3-(4-trifluoromethyl-phenyl)-acrylamide;
N—[(S)-1-Benzyl-2-((S)-3-dimethylamino-pyrrolidin-1-yl)-2-oxo-ethyl]-N-(4-pyridin-2-yl-benzyl)-3-(4-trifluoromethyl-phenyl)-acrylamide;
N—[(S)-1-Benzyl-2-((R)-3-dimethylamino-pyrrolidin-1-yl)-2-oxo-ethyl]-N-(4-pyridin-2-yl-benzyl)-3-(6-trifluoromethyl-pyridin-3-yl)-acrylamide;
N—[(S)-1-Benzyl-2-((S)-3-dimethylamino-pyrrolidin-1-yl)-2-oxo-ethyl]-N-(4-pyridin-2-yl-benzyl)-3-(6-trifluoromethyl-pyridin-3-yl)-acrylamide;
(S)—N-[1-Benzyl-2-(2,3-dihydro-indol-1-yl)-2-oxo-ethyl]-N-(4-pyrimidin-5-yl-benzyl)-3-(4-trifluoromethyl-phenyl)-acrylamide;
(S)—N-[1-Benzyl-2-(1,3-dihydro-isoindol-2-yl)-2-oxo-ethyl]-N-(4-pyridin-2-yl-benzyl)-3-(6-trifluoromethyl-pyridin-3-yl)-acrylamide;
(S)—N-{1-[(2-Methoxy-ethyl)-methyl-carbamoyl]-2-phenyl-ethyl}-N-(4-morpholin-4-yl-benzyl)-3-(1,3,5-Dimethyl-1H-pyrazol-4-yl)-acrylamide;
(S)-3-(2,4-Dimethyl-thiazol-5-yl)-N-{1-[(2-methoxy-ethyl)-methyl-carbamoyl]-2-phenyl-ethyl}-N-(4-morpholin-4-yl-benzyl)-acrylamide;
(S)—N-{1-[(2-Methoxy-ethyl)-methyl-carbamoyl]-2-phenyl-ethyl}-N-(4-morpholin-4-yl-benzyl)-3-(5-trifluoromethyl-pyridin-2-yl)-acrylamide;
(S)—N-{1-[(2-Methoxy-ethyl)-methyl-carbamoyl]-2-phenyl-ethyl}-3-(6-methoxy-pyridin-3-yl)-N-(4-morpholin-4-yl-benzyl)-acrylamide;
(S)-3-(2,5-Dimethyl-2H-pyrazol-3-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-N-(4-pyridin-2-yl-benzyl)-3-(1,3,5-trimethyl-1H-pyrazol-4-yl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(2-methyl-thiazol-4-yl)-N-(4-pyridin-2-yl-benzyl)-acrylamide;
(S)-3-(3,5-Dimethyl-isoxazol-4-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(6-methoxy-pyridazin-3-yl)-N-(4-pyridin-2-yl-benzyl)-acrylamide;
(S)—N-{1-[(2-Methoxy-ethyl)-methyl-carbamoyl]-2-phenyl-ethyl}-3-(6-methyl-pyridin-3-yl)-N-(4-pyridin-2-yl-benzyl)-acrylamide;
(S)-3-(6-Chloro-pyridin-3-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(5-methyl-pyridin-2-yl)-N-(4-pyridin-2-yl-benzyl)-acrylamide;
(S)—N-{1-[(2-Methoxy-ethyl)-methyl-carbamoyl]-2-phenyl-ethyl}-3-(6-methoxy-pyridin-3-yl)-N-(4-pyridin-2-yl-benzyl)-acrylamide;
(S)—N-{1-[(2-Methoxy-ethyl)-methyl-carbamoyl]-2-phenyl-ethyl}-N-(4-pyridin-2-yl-benzyl)-3-(2-trifluoromethyl-pyrimidin-5-yl)-acrylamide;
(S)—N-{1-[(2-Methoxy-ethyl)-methyl-carbamoyl]-2-phenyl-ethyl}-N-(4-pyridin-2-yl-benzyl)-3-(5-trifluoromethyl-pyridin-2-yl)-acrylamide;
(S)-3-(1,5-Dimethyl-1H-pyrazol-4-yl)-N-{1-[(2-methoxy-ethyl)-methyl-carbamoyl]-2-phenyl-ethyl}-N-(4-pyridin-2-yl-benzyl)-acrylamide;
(S)—N-{1-[(2-Methoxy-ethyl)-methyl-carbamoyl]-2-phenyl-ethyl}-3-(1-methyl-1H-pyrazol-3-yl)-N-(4-pyridin-4-yl-benzyl)-acrylamide;
(S)-3-(2,3-Dimethyl-3H-imidazol-4-yl)-N-{1-[(2-methoxy-ethyl)-methyl-carbamoyl]-2-phenyl-ethyl}-N-(4-pyridin-4-yl-benzyl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyridin-4-yl-benzyl)-acrylamide;
(S)—N-{1-[(2-Methoxy-ethyl)-methyl-carbamoyl]-2-phenyl-ethyl}-3-(2-methyl-thiazol-4-yl)-N-(4-pyridin-4-yl-benzyl)-acrylamide;
(S)—N-{1-[(2-Methoxy-ethyl)-methyl-carbamoyl]-2-phenyl-ethyl}-3-(5-methyl-isoxazol-3-yl)-N-(4-pyridin-4-yl-benzyl)-acrylamide;
(S)-3-(3,5-Dimethyl-isoxazol-4-yl)-N-{1-[(2-methoxy-ethyl)-methyl-carbamoyl]-2-phenyl-ethyl}-N-(4-pyridin-4-yl-benzyl)-acrylamide;
(S)-3-(2,5-Dimethyl-oxazol-4-yl)-N-{1-[(2-methoxy-ethyl)-methyl-carbamoyl]-2-phenyl-ethyl}-N-(4-pyridin-4-yl-benzyl)-acrylamide;
(S)—N-{1-[(2-Methoxy-ethyl)-methyl-carbamoyl]-2-phenyl-ethyl}-N-(4-pyridin-4-yl-benzyl)-3-[1,2,3]thiadiazol-4-yl-acrylamide;
(S)-3-(6-Chloro-pyridin-3-yl)-N-{1-[(2-methoxy-ethyl)-methyl-carbamoyl]-2-phenyl-ethyl}-N-(4-pyridin-4-yl-benzyl)-acrylamide;
(S)—N-{1-[(2-Methoxy-ethyl)-methyl-carbamoyl]-2-phenyl-ethyl}-3-(5-methyl-pyridin-2-yl)-N-(4-pyridin-4-yl-benzyl)-acrylamide;
(S)—N-{1-[(2-Methoxy-ethyl)-methyl-carbamoyl]-2-phenyl-ethyl}-3-(6-methoxy-pyridin-3-yl)-N-(4-pyridin-4-yl-benzyl)-acrylamide;
(S)—N-{1-[(2-Methoxy-ethyl)-methyl-carbamoyl]-2-phenyl-ethyl}-N-(4-pyridin-4-yl-benzyl)-3-(5-trifluoromethyl-pyridin-2-yl)-acrylamide;
(S)—N-[4-(4-Acetyl-piperazin-1-yl)-benzyl]-3-(2,5-dimethyl-2H-pyrazol-3-yl)-N-{1-[(2-methoxy-ethyl)-methyl-carbamoyl]-2-phenyl-ethyl}-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(1,3,5-trimethyl-1H-pyrazol-4-yl)-acrylamide;
(S)—N-[4-(4-Acetyl-piperazin-1-yl)-benzyl]-3-(2,4-dimethyl-thiazol-5-yl)-N-{1-[(2-methoxy-ethyl)-methyl-carbamoyl]-2-phenyl-ethyl}-acrylamide;
(S)—N-[4-(4-Acetyl-piperazin-1-yl)-benzyl]-3-(6-chloro-pyridin-3-yl)-N-{1-[(2-methoxy-ethyl)-methyl-carbamoyl]-2-phenyl-ethyl}-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(6-methoxy-pyridin-3-yl)-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(5-trifluoromethyl-pyridin-2-yl)-acrylamide;
(S)—N-{1-[(2-Methoxy-ethyl)-methyl-carbamoyl]-2-phenyl-ethyl}-3-(1-methyl-1H-pyrazol-3-yl)-N-(4-pyrimidin-5-yl-benzyl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyrimidin-5-yl-benzyl)-acrylamide;
(S)-3-(5-Chloro-pyridin-2-yl)-N-{1-[(2-methoxy-ethyl)-methyl-carbamoyl]-2-phenyl-ethyl}-N-(4-pyrimidin-5-yl-benzyl)-acrylamide;
(S)-3-(6-Chloro-pyridin-3-yl)-N-{1-[(2-methoxy-ethyl)-methyl-carbamoyl]-2-phenyl-ethyl}-N-(4-pyrimidin-5-yl-benzyl)-acrylamide;
(S)—N-{1-[(2-Methoxy-ethyl)-methyl-carbamoyl]-2-phenyl-ethyl}-3-(2-methoxy-pyrimidin-5-yl)-N-(4-pyrimidin-5-yl-benzyl)-acrylamide;
(S)—N-{1-[(2-Methoxy-ethyl)-methyl-carbamoyl]-2-phenyl-ethyl}-N-(4-pyrimidin-5-yl-benzyl)-3-(5-trifluoromethyl-pyridin-2-yl)-acrylamide;
(S)-3-(2,4-Dimethyl-thiazol-5-yl)-N-{1-[(2-methoxy-ethyl)-methyl-carbamoyl]-2-phenyl-ethyl}-N-(4-pyridin-3-yl-benzyl)-acrylamide;
(S)-3-(2,5-Dimethyl-oxazol-4-yl)-N-{1-[(2-methoxy-ethyl)-methyl-carbamoyl]-2-phenyl-ethyl}-N-(4-pyridin-3-yl-benzyl)-acrylamide;
(S)-3-(6-Chloro-pyridin-3-yl)-N-{1-[(2-methoxy-ethyl)-methyl-carbamoyl]-2-phenyl-ethyl}-N-(4-pyridin-3-yl-benzyl)-acrylamide;
(S)—N-{1-[(2-Methoxy-ethyl)-methyl-carbamoyl]-2-phenyl-ethyl}-3-(6-methoxy-pyridin-3-yl)-N-(4-pyridin-3-yl-benzyl)-acrylamide;
(S)—N-[1-Benzyl-2-(4-dimethylamino-piperidin-1-yl)-2-oxo-ethyl]-N-(6-morpholin-4-yl-pyridin-3-ylmethyl)-3-(4-trifluoromethyl-phenyl)-acrylamide;
(S)—N-[1-Benzyl-2-(4-benzyl-piperidin-1-yl)-2-oxo-ethyl]-N-(6-morpholin-4-yl-pyridin-3-ylmethyl)-3-(4-trifluoromethyl-phenyl)-acrylamide;
(S)—N-[1-Benzyl-2-(4-benzyl-piperidin-1-yl)-2-oxo-ethyl]-N-(6-morpholin-4-yl-pyridin-3-ylmethyl)-3-(6-trifluoromethyl-pyridin-3-yl)-acrylamide;
(S)—N-[1-Benzyl-2-(4-benzyl-piperidin-1-yl)-2-oxo-ethyl]-3-(4-methoxy-phenyl)-N-(6-morpholin-4-yl-pyridin-3-ylmethyl)-acrylamide;
(S)—N-[1-Benzyl-2-(4-benzyl-piperidin-1-yl)-2-oxo-ethyl]-N-(6-morpholin-4-yl-pyridin-3-ylmethyl)-3-p-tolyl-acrylamide;
(S)—N-(1-Benzyl-2-morpholin-4-yl-2-oxo-ethyl)-N-(6-morpholin-4-yl-pyridin-3-ylmethyl)-3-p-tolyl-acrylamide;
(S)—N-(1-Benzyl-2-morpholin-4-yl-2-oxo-ethyl)-3-(4-methoxy-phenyl)-N-(6-morpholin-4-yl-pyridin-3-ylmethyl)-acrylamide;
(S)—N-[1-Benzyl-2-(5,8-dihydro-6H-[1,7]naphthyridin-7-yl)-2-oxo-ethyl]-N-(4-pyridin-2-yl-benzyl)-3-(4-trifluoromethyl-phenyl)-acrylamide;
(S)—N-{1-[(2-Methoxy-ethyl)-methyl-carbamoyl]-2-phenyl-ethyl}-N-(6-morpholin-4-yl-pyridin-3-ylmethyl)-3-(4-trifluoromethyl-phenyl)-acrylamide;
(S)—N-{1-[(2-Methoxy-ethyl)-methyl-carbamoyl]-2-phenyl-ethyl}-N-(6-morpholin-4-yl-pyridin-3-ylmethyl)-3-p-tolyl-acrylamide;
(S)—N-{1-[(2-Methoxy-ethyl)-methyl-carbamoyl]-2-phenyl-ethyl}-3-(4-methoxy-phenyl)-N-(6-morpholin-4-yl-pyridin-3-ylmethyl)-acrylamide;
(S)—N-Benzyl-N-{1-[(2-methoxy-ethyl)-methyl-carbamoyl]-2-phenyl-ethyl}-3-p-tolyl-acrylamide;
(S)—N-(4-Ethyl-benzyl)-N-{1-[(2-methoxy-ethyl)-methyl-carbamoyl]-2-phenyl-ethyl}-3-p-tolyl-acrylamide;
(S)—N-{1-[(2-Methoxy-ethyl)-methyl-carbamoyl]-2-phenyl-ethyl}-N-pyridin-2-ylmethyl-3-p-tolyl-acrylamide;
(S)—N-{1-[(2-Methoxy-ethyl)-methyl-carbamoyl]-2-pyrrol-1-yl-ethyl}-N-(4-pyridin-2-yl-benzyl)-3-(4-trifluoromethyl-phenyl)-acrylamide;
N-[1-[(2-Methoxy-ethyl)-methyl-carbamoyl]-2-(1-methyl-1H-pyrazol-3-yl)-ethyl]-N-(4-pyridin-2-yl-benzyl)-3-(4-trifluoromethyl-phenyl)-acrylamide;
N-[1-[(2-Methoxy-ethyl)-methyl-carbamoyl]-2-(1-methyl-1H-pyrazol-4-yl)-ethyl]-N-(4-pyridin-4-yl-benzyl)-3-(4-trifluoromethyl-phenyl)-acrylamide;
N-[1-[(2-Methoxy-ethyl)-methyl-carbamoyl]-2-(1-methyl-1H-pyrazol-3-yl)-ethyl]-N-(4-pyridin-4-yl-benzyl)-3-(4-trifluoromethyl-phenyl)-acrylamide;
(S)—N-{1-[(2-Dimethylamino-ethyl)-methyl-carbamoyl]-2-phenyl-ethyl}-N-(4-pyridin-2-yl-benzyl)-3-(4-trifluoromethyl-phenyl)-acrylamide;
N-{1-[((S)-1-Benzyl-pyrrolidin-3-yl)-methyl-carbamoyl]-(S)-2-phenyl-ethyl}-N-(4-pyridin-2-yl-benzyl)-3-(4-trifluoromethyl-phenyl)-acrylamide;
N-{1-[((R)-1-Benzyl-pyrrolidin-3-yl)-methyl-carbamoyl]-(S)-2-phenyl-ethyl}-N-(4-pyridin-2-yl-benzyl)-3-(4-trifluoromethyl-phenyl)-acrylamide;
(S)—N-{1-[(2-Hydroxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-{1-[(2-hydroxy-ethyl)-methyl-carbamoyl]-2-phenyl-ethyl}-3-(4-trifluoromethyl-phenyl)-acrylamide;
(S)—N-{1-[(4-Methoxy-benzyl)-methyl-carbamoyl]-2-phenyl-ethyl}-N-(4-morpholin-4-yl-benzyl)-3-(6-trifluoromethyl-pyridin-3-yl)-acrylamide;
(S)—N-[4-(4-Acetyl-piperazin-1-yl)-benzyl]-N-{1-[(2-methoxy-ethyl)-methyl-carbamoyl]-2-phenyl-ethyl}-3-(4-trifluoromethyl-phenyl)-acrylamide;
(S)—N-{1-[(2-Methoxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-(1-{methyl-[2-(pyrimidin-2-yloxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-benzyloxy-ethyl)-methyl-carbamoyl]-2-phenyl-ethyl}-3-(4-trifluoromethyl-phenyl)-acrylamide;
10 (S)—N-{1-[(2-Benzyloxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-(1-{[2-(2,4-dimethyl-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(3-fluoro-4-methoxy-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(3-cyano-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(3-trifluoromethoxy-benzyloxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(3-methoxy-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(4-difluoromethoxy-benzyloxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(1-methyl-1H-benzotriazol-5-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(pyridin-3-ylmethoxy)-ethyl]-carbamoyl}-2-phenyl-ethyl)-3-(4-trifluoromethyl-phenyl)-acrylamide;
(S)—N-(1-{[2-(3-Methoxy-benzyloxy)-ethyl]methyl-carbamoyl}-2-phenyl-ethyl)-N-(4-morpholin-4-yl-benzyl)-3-(4-trifluoromethyl-phenyl)-acrylamide;
(S)—N-{1-[(2-Ethoxy-ethyl)-methyl-carbamoyl]-2-phenyl-ethyl}-N-(4-morpholin-4-yl-benzyl)-3-(4-trifluoromethyl-phenyl)-acrylamide;
(S)—N-[4-(4-Acetyl-piperazin-1-yl)-benzyl]-N-{1-[(2-ethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-(1-{[2-(2,4-dimethyl-benzyloxy)-ethyl]-methyl-carbamoyl}-2-phenyl-ethyl)-3-(4-trifluoromethyl-phenyl)-acrylamide;
(S)—N-(1-{[2-(2,4-Dimethyl-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(3-Fluoro-4-methoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(3-Cyano-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{Methyl-[2-(3-trifluoromethoxy-benzyloxy)-ethyl]-carbamoyl}-2-phenyl-ethyl)-N-(4-morpholin-4-yl-benzyl)-3-(4-trifluoromethyl-phenyl)-acrylamide;
(S)—N-(1-{[2-(3,5-Dimethoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(3-Methoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{[2-(4-Difluoromethoxy-benzyloxy)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-(Methyl-[2-(1-methyl-1H-benzotriazol-5-ylmethoxy)-ethyl]-carbamoyl)-2-phenyl-ethyl)-N-(4-morpholin-4-yl-benzyl)-3-(4-trifluoromethyl-phenyl)-acrylamide;
(S)—N-(1-{Methyl-[2-(pyridin-3-ylmethoxy)-ethyl]-carbamoyl}-2-phenyl-ethyl)-N-(4-morpholin-4-yl-benzyl)-3-(4-trifluoromethyl-phenyl)-acrylamide;
(S)—N-[4-(4-Acetyl-piperazin-1-yl)-benzyl]-N-(1-{methyl-[2-(5-methyl-isoxazol-3-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(pyridin-4-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(5-trifluoromethyl-furan-2-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-cyclopropylmethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-(1-{methyl-[2-(pyridin-4-ylmethoxy)-ethyl]-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-(1-{methyl-[2-(5-methyl-isoxazol-3-ylmethoxy)-ethyl]-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-(1-{[2-(2-benzyloxy-ethoxy)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-cyanomethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-{1-[(2-allyloxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-{1-[(2-carbamoylmethoxy-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-(1-{methyl-[2-(pyridin-2-ylmethoxy)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(pyridin-2-ylmethoxy)-ethyl]-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-(1-{[2-(2-benzyloxy-ethoxy)-ethyl]-methyl-carbamoyl}-2-phenyl-ethyl)-3-(6-trifluoromethyl-pyridin-3-yl)-acrylamide;
(S)—N-[4-(4-Acetyl-piperazin-1-yl)-benzyl]-N-{1-[(2-cyanomethoxy-ethyl)-methyl-carbamoyl]-2-phenyl-ethyl}-3-(6-trifluoromethyl-pyridin-3-yl)-acrylamide;
(S)—N-[4-(4-Acetyl-piperazin-1-yl)-benzyl]-N-{1-[(2-allyloxy-ethyl)-methyl-carbamoyl]-2-phenyl-ethyl}-3-(6-trifluoromethyl-pyridin-3-yl)-acrylamide;
(S)—N-[1-({2-[(5-Bromo-furan-2-ylmethyl)-methyl-amino]ethyl}-methyl-carbamoyl)-2-phenyl-ethyl]-N-(4-pyridin-2-yl-benzyl)-3-(4-trifluoromethyl-phenyl)-acrylamide;
(S)—N-(1-{[2-(Benzyl-methyl-amino)-ethyl]methyl-carbamoyl}-2-phenyl-ethyl)-N-(4-pyridin-2-yl-benzyl)-3-(4-trifluoromethyl-phenyl)-acrylamide;
(S)—N-[1-({2-[(6-Chloro-pyridin-3-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-(1-{[2-(Furan-3-ylmethyl-methyl-amino)-ethyl]-methyl-carbamoyl}-2-phenyl-ethyl)-N-(4-pyridin-2-yl-benzyl)-3-(4-trifluoromethyl-phenyl)-acrylamide;
(S)—N-(1-{[2-(Furan-2-ylmethyl-methyl-amino)-ethyl]-methyl-carbamoyl}-2-phenyl-ethyl)-N-(4-pyridin-2-yl-benzyl)-3-(4-trifluoromethyl-phenyl)-acrylamide;
(S)—N-(1-{Methyl-[2-(methyl-pyridin-2-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-(1-{Methyl-[2-(methyl-thiophen-2-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-[1-({2-[(5-Chloro-thiophen-2-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(6-Bromo-pyridin-3-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(5-Hydroxymethyl-furan-2-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(6-Methoxy-pyridin-3-ylmethyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-(Methyl-{2-[methyl-(6-trifluoromethyl-pyridin-3-ylmethyl)-amino]-ethyl}-carbamoyl)-2-phenyl-ethyl]-N-(4-pyridin-2-yl-benzyl)-3-(4-trifluoromethyl-phenyl)-acrylamide;
(S)—N-(1-{Methyl-[2-(methyl-pyridin-3-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-(1-{Methyl-[2-(methyl-thiophen-3-ylmethyl-amino)-ethyl]-carbamoyl}-2-phenyl-ethyl)-N-(4-pyridin-2-yl-benzyl)-3-(4-trifluoromethyl-phenyl)-acrylamide;
(S)—N-[1-(Methyl-{2-[methyl-(2-methyl-benzyl)-amino]-ethyl}-carbamoyl)-2-phenyl-ethyl]-N-(4-pyridin-2-yl-benzyl)-3-(4-trifluoromethyl-phenyl)-acrylamide;
(S)—N-[1-({2-[(2,4-Dimethyl-benzyl)-methyl-amino]-ethyl}-methyl-carbamoyl)-2-phenyl-ethyl]-N-(4-pyridin-2-yl-benzyl)-3-(4-trifluoromethyl-phenyl)-acrylamide;
(S)—N-[1-({2-[(3,5-Dimethoxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[4-(4-Acetyl-piperazin-1-yl)-benzyl]-N-[1-({2-[(3,5-dimethoxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{[2-({4-[(2-hydroxy-ethyl)-methyl-amino]-benzyl}-methyl-amino)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-[1-({2-[(4-hydroxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(4-diethylamino-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(3-hydroxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{methyl-[2-(methyl-pyridin-3-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-{1-[(2-{[3-(2-hydroxy-ethoxy)-benzyl]-methyl-amino}-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-[1-({2-[(3-cyano-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(4-isopropoxy-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-(methyl-{2-[methyl-(4-methyl-3,4-dihydro-2H-benzo[1,4]oxazin-7-ylmethyl)-amino]-ethyl}-carbamoyl)-2-phenyl-ethyl]-3-(4-trifluoromethyl-phenyl)-acrylamide;
(S)—N-[4-(4-Acetyl-piperazin-1-yl)-benzyl]-N-{1-[(2-{[4-(3-dimethylamino-propoxy)-benzyl]-methyl-amino}-ethyl)-methyl-carbamoyl]-2-phenyl-ethyl}-3-(4-trifluoromethyl-phenyl)-acrylamide;
(S)—N-[4-(4-Acetyl-piperazin-1-yl)-benzyl]-N-[1-({2-[(6-methoxy-pyridin-3-ylmethyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{methyl-[2-(methyl-thiazol-2-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(benzo[1,3]dioxol-5-ylmethyl-methyl-amino)-ethyl]-methyl-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{methyl-[2-(methyl-pyrimidin-5-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-[1-({2-[(2,3-difluoro-4-methyl-benzyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-[1-({2-[(3H-imidazol-4-ylmethyl)-methyl-amino]-ethyl}-methyl-carbamoyl)-2-phenyl-ethyl]-3-(4-trifluoromethyl-phenyl)-acrylamide;
(S)—N-[4-(4-Acetyl-piperazin-1-yl)-benzyl]-N-(1-{methyl-[2-(methyl-pyridin-4-ylmethyl-amino)-ethyl]-carbamoyl}-2-phenyl-ethyl)-3-(4-trifluoromethyl-phenyl)-acrylamide;
(S)—N-[4-(4-Acetyl-piperazin-1-yl)-benzyl]-N-(1-{[2-(benzyl-methyl-amino)-ethyl]-methyl-carbamoyl}-2-phenyl-ethyl)-3-(4-trifluoromethyl-phenyl)-acrylamide;
(S)—N-(1-{[2-({4-[(2-Hydroxy-ethyl)-methyl-amino]-benzyl}-methyl-amino)-ethyl]-methyl-carbamoyl}-2-phenyl-ethyl)-N-(4-morpholin-4-yl-benzyl)-3-(4-trifluoromethyl-phenyl)-acrylamide;
(S)—N-[1-({2-[(4-Hydroxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(4-Diethylamino-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(3-Hydroxy-benzyl)-methyl-amino]ethyl}-methyl-carbamoyl)-2-phenyl-ethyl]-N-(4-morpholin-4-yl-benzyl)-3-(4-trifluoromethyl-phenyl)-acrylamide;
(S)—N-(1-{Methyl-[2-(methyl-pyridin-3-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-{1-[(2-{[3-(2-Hydroxy-ethoxy)-benzyl]-methyl-amino}-ethyl)-methyl-carbamoyl]-2-phenyl-ethyl}-N-(4-morpholin-4-yl-benzyl)-3-(4-trifluoromethyl-phenyl)-acrylamide;
(S)—N-[1-({2-[(3-Cyano-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(4-Isopropoxy-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-(Methyl-{2-[methyl-(4-methyl-3,4-dihydro-2H-benzo[1,4]oxazin-7-ylmethyl)-amino]-ethyl}-carbamoyl)-2-phenyl-ethyl]-N-(4-morpholin-4-yl-benzyl)-3-(4-trifluoromethyl-phenyl)-acrylamide;
(S)—N-[1-({2-[(6-Methoxy-pyridin-3-ylmethyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-(1-{Methyl-[2-(methyl-thiazol-2-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-(1-{[2-(Benzo[1,3]dioxol-5-ylmethyl-methyl-amino)-ethyl]-methyl-carbamoyl}-2-phenyl-ethyl)-N-(4-morpholin-4-yl-benzyl)-3-(4-trifluoromethyl-phenyl)-acrylamide;
(S)—N-(1-{Methyl-[2-(methyl-pyrimidin-5-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-[1-({2-[(2,3-Difluoro-4-methyl-benzyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-[1-({2-[(3H-Imidazol-4-ylmethyl)-methyl-amino]-ethyl}-methyl-carbamoyl)-2-phenyl-ethyl]-N-(4-morpholin-4-yl-benzyl)-3-(4-trifluoromethyl-phenyl)-acrylamide;
(S)—N-(1-{Methyl-[2-(methyl-pyridin-4-ylmethyl-amino)-ethyl]-carbamoyl}-2-phenyl-ethyl)-N-(4-morpholin-4-yl-benzyl)-3-(4-trifluoromethyl-phenyl)-acrylamide;
(S)—N-(1-{Methyl-[4-(2-oxo-pyrrolidin-1-yl)-benzyl]-carbamoyl}-2-phenyl-ethyl)-N-(4-morpholin-4-yl-benzyl)-3-(6-trifluoromethyl-pyridin-3-yl)-acrylamide;
(S)-Cyclopropanecarboxylic acid (4-{[methyl-(2-{(4-morpholin-4-yl-benzyl)-[3-(6-trifluoromethyl-pyridin-3-yl)-acryloyl]-amino}-3-phenyl-propionyl)-amino]-methyl}-phenyl)-amide;
(S)—N-(1-{Methyl-[4-(2-oxo-piperidin-1-yl)-benzyl]-carbamoyl}-2-phenyl-ethyl)-N-(4-morpholin-4-yl-benzyl)-3-(6-trifluoromethyl-pyridin-3-yl)-acrylamide;
(S)—N-(4-{[Methyl-(2-{(4-morpholin-4-yl-benzyl)-[3-(6-trifluoromethyl-pyridin-3-yl)-acryloyl]amino}-3-phenyl-propionyl)-amino]-methyl}-phenyl)-benzamide;
(S)—N-(1-{[4-(Acetyl-phenyl-amino)-benzyl]-methyl-carbamoyl}-2-phenyl-ethyl)-N-(4-morpholin-4-yl-benzyl)-3-(6-trifluoromethyl-pyridin-3-yl)-acrylamide;
(S)—N-(1-{[4-(2-Methoxy-ethylamino)-benzyl]-methyl-carbamoyl}-2-phenyl-ethyl)-N-(4-morpholin-4-yl-benzyl)-3-(6-trifluoromethyl-pyridin-3-yl)-acrylamide;
N-{1-[(2-Methoxy-ethyl)-methyl-carbamoyl]-2-morpholin-4-yl-ethyl}-N-(4-pyridin-2-yl-benzyl)-3-(4-trifluoromethyl-phenyl)-acrylamide; and
N-{1-[(2-Methoxy-ethyl)-methyl-carbamoyl]-2-pyrrol-1-yl-ethyl}-N-(4-pyridin-4-yl-benzyl)-3-(4-trifluoromethyl-phenyl)-acrylamide,
in a free or a pharmaceutically acceptable salt form.

12. The pharmaceutical composition comprising a compound according to claim 1, in a free or a pharmaceutically acceptable salt form, and a pharmaceutically acceptable carrier material.

13. (canceled)

14. A method of treatment or prophylaxis of a disease or disorder associated with protozoal infections, wherein said method comprises administering to a subject in need thereof an effective amount of the compound according to claim 1.

15. The method of treatment or prophylaxis of a disease or disorder according to claim 14, wherein said disease or disorder associated with protozoal infections is malaria.
